(19) United States
(12) Patent Application Publication
(10) Pub. No.: US 2023/0399706 A1
(43) Pub. Date: Dec. 14, 2023

(54) Method of Pooled Sample Pathogen Testing for Use in Screening Large Groups of Individuals

(71) Applicant: David A. Metzger, Cedarburg, WI (US)

(72) Inventor: David A. Metzger, Cedarburg, WI (US)

(21) Appl. No.: 17/829,353

(22) Filed: May 31, 2022

Related U.S. Application Data
(60) Provisional application No. 63/194,340, filed on May 28, 2021.
(60) Provisional application No. 63/278,783, filed on Nov. 12, 2021.

(51) Int. Cl. C12Q 1/70; C12Q 1/6806; A61B 10/00

(52) U.S. Cl. CPC C12Q 1/701; C12Q 1/6806; A61B 10/0051; A61F 13/38

(57) ABSTRACT

A method of testing for disease through pooled samples. The individual to be tested may give one or more biological samples, where a biological sample from the individual may be combined with biological samples from other individuals to create a pool of samples within a liquid media. In one preferred method, the liquid media containing the combined or pooled biological samples are concentrated to a low volume. The invention also is directed to a system and method of pooled coronavirus testing for testing and detecting the presence or absence of coronavirus in pooled groups of individuals in pool sizes of 25, 50, 100, 150, 200, 250, 500, 1000 and greater.

### CROSS REFERENCE TO RELATED APPLICATIONS

Pursuant to 35 U.S.C. § 119(e), this application claims all benefits to and priority in U.S. Provisional Application Ser. No. 63/194,340, filed on May 28, 2021, and U.S. Provisional Application Ser. No. 63/278,783, filed on Nov. 12, 2021, the entirety of both of which are hereby expressly incorporated by reference herein.

### SEQUENCE LISTING

The present application contains a Sequence Listing which has been submitted electronically in ASCII format as a file named Pooling_Project_ST25.TXT created Aug. 21, 2023, which is 1,191 bytes in size, and which is incorporated herein by reference in its entirety.

### FIELD OF THE INVENTION

The present invention is directed towards methods of testing for a pathogen, and more particularly a method of screening for a disease-causing pathogen in individuals by testing pooled biological samples from such individuals by processing the pooled sample into a refined pooled testing sample that increases the accuracy and robustness of detection of a pathogen in the pooled sample by diagnostic testing of the concentrated pooled sample.

### BACKGROUND OF THE INVENTION

Diseases can jump from animals to humans or mutate, causing unknown diseases to infect humans. For example, one such disease is COVID-19, which causes respiratory infections. An individual's symptoms caused by COVID-19 can range from asymptomatic to acute respiratory distress syndrome and even death. COVID-19 is highly contagious which makes rapid, accurate identification of infected individuals a major public health concern so that infected individuals can quarantine themselves and prevent further spreading of the disease. While asymptomatic individuals may lack symptoms, those infected individuals can still transmit the disease.

Thus, one important tool to prevent disease transmission is testing individuals to determine whether people have been infected so that they can quarantine and prevent further transmission of disease. Surveillance testing can also be important in monitoring a community or characterizing an occurrence of disease to better understand and determine disease management (e.g., determine whether lock-downs should be implemented or if masks should be worn). Surveillance testing can involve testing a certain percentage of a specific population to monitor for increasing or decreasing prevalence or to determine the effect of community interventions such as social distancing.

It is expensive and time consuming, however, to test each individual sample. It would therefore be beneficial to pool samples and test the resulting pool. Pooling samples involves mixing several samples together in a “batch” or pooled sample, then testing the pooled sample with a diagnostic test. Pooling samples can save both time and resources. Pooling samples, however, have their own drawbacks because the samples are diluted. The accuracy and difficulties only increase when the number of pooled samples increase because there is less viral genetic material available to detect, and therefore is a greater likelihood of false negative results. It is therefore desirable for an accurate method for bulk testing biological samples.

What is needed is a method of pooled testing that is fast, efficient, easy to administrate, and which produces accurate and repeatable results for relatively large pools of test subjects.

### SUMMARY OF THE INVENTION

The present invention is directed to a method of pooled diagnostic testing for a known pathogen of a relatively large number of individuals constituting members of a pool in which a plurality of test samples is taken from each pool member where one set of the samples from each member are pooled together to produce a single pooled sample which in turn is processed into a pooled test sample that provides more consistently more repeatably accurate test results In a preferred method and embodiment, the pooled sample is processed using a method configured to remove excess fluid from the sample while retaining a greater percentage of any pathogen being tested in the reduced fluid volume pathogen detection enhanced pooled test sample that is then tested for the presence of the pathogen. Where a pooled test sample returns a positive test result that is indicative of the presence of the specific pathogen being tested, a second set of test samples from the pool members are individually tested to determine specifically which member or members of the pool are positive for the pathogen being tested. Where the pathogen detection enhanced pooled test sample returns a negative test result, it advantageously reduces the amount of resources, time and money spent on pathogen testing by eliminating the need to perform a separate diagnostic test on each member.

The present invention is directed to at least one implementation of a method of pooled testing for a pathogen that includes: (a) collecting a sample from each one of at least a plurality of pairs, i.e., at least three, of individuals, i.e., members, that make up a testing pool; (b) pooling all of the samples from all of the individuals of the pool into a single pooled sample; (c) processing the pooled sample to prepare the pooled sample for performing a diagnostic test thereon; and (d) performing a diagnostic test on the processed pooled sample to obtain a test result indicative of the presence or absence of the pathogen, the test result being either (i) a positive rest result indicating the presence of the pathogen in one or more of the individuals of the testing pool, or (ii) a negative test result indicating the absence of the pathogen in any of the individuals of the testing pool. During the collection step (a) a second sample also is collected from each one of the same members of the testing pool, with this second sample stored separately from the pooled sample so that each second sample can be separately tested if the pooled sample tests positive to determine which one or more members of the testing pool test positive and carry the pathogen.

Each one of the samples can be and preferably is taken or obtained nasally, e.g., via a nasal specimen, bronchially, e.g., via a bronchial specimen, or via an oropharyngeal sample, e.g., via a throat specimen. Where the samples are obtained by taking oropharyngeal samples, the oropharyngeal samples can be nasopharyngeal samples, e.g., specimens taken from the oropharynx, oropharyngeal samples, e.g., specimens taken from the oropharynx, and/or hypopharyngeal samples, e.g., specimens taken from the hypopharynx of each person of the pool. Each one of the samples can also be a sample obtained from aspirate or a wash including from nasal aspirate, a nasal wash, nasopharyngeal aspirate, a nasopharyngeal wash, endotracheal aspirate, an endotracheal wash, a bronchoalveolar lavage (BAL), a bronchial wash, a throat wash, or another type of nasal and/or upper respiratory system wash.

In one preferred implementation of a method of pooled pathogen testing of the present invention, a swab is used to collect each one of the samples from each member of the testing pool. In one such preferred method implementation, a nasal swab is used to swab at least one of the nostrils and preferably a separate nasal swab is used to swab each one of the nostrils of each one of the individuals of the pool. If desired, swabs can be used to collect bronchial specimens, oropharyngeal specimens, as well as to collect specimens from another other biological site, i.e., in vivo site, of the members of the pool during step (a) of the method. If desired and depending upon the specific type of specimen collecting method used to obtain the samples from the members of the pool, each swab used can be a nasopharyngeal swab, a mid-turbinate swab, a foam tipped oral swab, an anterior nares/nasal swab, or an oropharyngeal swab.

In a preferred implementation of a method of pooled pathogen testing of the present invention, at least two samples are taken from each member of the testing pool, with (i) one sample of each member of the pool collectively defining a pooled sample subset of all of the samples collected from the pool members during the sampling session that are pooled together into a common pooled pathogen sample upon which a pooled pathogen test is performed to determine whether any member(s) of the pool have, e.g., are infected with, the pathogen, and (ii) another sample of each member of the pool collectively defining a pathogen verification sample subset of all of the samples collected from the pool members during the sampling session which remain separate from each other and which are stored separately from each other to each be tested to determine which member(s) of the pool have, e.g., are infected with, the pathogen upon a positive test result obtained from testing the pooled sample. At least the samples or specimens of the pathogen verification sample subset individually or separately stored, such as in a refrigerator, temperature, climate and/or humidity-controlled vault, or another type of pathogen sample storage chamber configured for stable multiple week, i.e., at least a plurality of weeks, and preferably multiple month, i.e., at least a plurality of months, sample or specimen retention, and which preferably also is lockable or otherwise secure, so that each individual sample or specimen is ready to be separately or individually tested upon a positive test result obtained from the pooled sample.

In a preferred implementation of the pooled pathogen testing method, all of the samples from each one of the members of the pool of the pooled sample subset are placed in a single container or a single bag for subsequent pooled pathogen testing on the pooled sample. In one preferred pooled pathogen testing method implementation, all of the samples from all of the pool members that makeup the pooled sample subset are placed in a single container or single bag to which no liquid, e.g., no water, nor any solution, e.g., no aqueous solution, is added. In one such preferred method implementation, the single container or single bag contains no liquid nor any solution prior to all of the samples being pooled together in the single container or single bag and no liquid nor any solution is added after all of the samples have been pooled together in the single container or single bag. All of the samples of the pooled sample subset are preferably placed in the single container or single bag during the same specimen collecting session where all of the samples are being collected from all of the pool members with all of the samples of the pooled sample subset placed in the single container or single bag while at the locus or location of the testing site where the specimen collecting session is carried out to obtain samples from all of the pool members. As such, the pooling of the samples in method step (b) is performed at a site or location where the samples are taken from the members of the testing pool that preferably is a common site or location where all of the samples are taken from all of the members of the pool during the same sample collecting session.

In a preferred pooled sample pathogen testing method implementation, the sample collecting step (a) includes performing the following substeps for each individual of the testing pool: (1) collecting a biological sample from the individual using a sample collector that is placed in a sample holding fluid medium (viral transport medium) in a sterile sample holding container, (2) transferring at least part of the biological sample from the sample collector to the sample holding fluid media in the sample holding container, and (3) sealing the sample holding container. A preferred sample collector is or includes a swab that is used to swab by frictionally engaging at least one and preferably both nostrils of the individual of the pool whose sample is being taken, the sample holding fluid media is or includes a saline solution in the sample holding container, during transferring of at least part of the sample from the swab to the saline solution, at least one of the swab, saline solution and sample holding container are agitated to cause transfer of at least part of the sample from the swab to the saline solution in the sample holding container, and thereafter the swab is removed from the sample holding container before the container is sealed. In a preferred sample collecting method implementation, the swab, the saline solution and the sample holding container are all agitated together or substantially simultaneously in preparing or readying the pooled sample for diagnostic or pathogen testing.

In carrying out a preferred implementation of the pool pathogen testing method, the sample processing step (c) includes performing the following substeps: (1) preparing the pooled sample containing sample from each one of the individuals of the testing pool, (2) extracting ribonucleic acid (RNA) from cells in the pooled sample, (3) purifying the pooled RNA, and (4) eluting the purified pooled RNA. In at least one pathogen testing method implementation, the sample processing step (c) further includes performing the following additional substep of producing a diagnostic testing ready purified pooled RNA containing mixture composed of (i) the purified pooled RNA and (ii) a solution that includes a buffer, reverse transcriptase, nucleotides (dNTPs), a forward primer, a reverse primer, a probe, and a DNA polymerase, and wherein the diagnostic testing step (d) includes performing a PCR diagnostic test using the diagnostic testing ready purified pooled RNA containing mixture. The present invention therefore includes a PCR diagnostic test that uses the diagnostic testing ready purified pooled RNA containing mixture in determining whether the pooled sample is positive or negative in carrying out the diagnostic testing step (d).

The PCR diagnostic test preferably is configured to determine whether a coronavirus, preferably SARS-CoV-2 genetic material, is present in any of the RNA in the diagnostic testing ready purified pooled RNA containing mixture during carrying out step (d). In a preferred embodiment and method implementation, the PCR diagnostic test is configured to determine whether a sufficient amount of SARS-CoV-2 genetic material is present in the RNA in the diagnostic testing ready purified pooled RNA containing mixture to produce a positive PCR diagnostic test result in step (d). In another preferred embodiment, the PCR diagnostic test is configured to determine whether a great enough viral load of SARS-CoV-2 is present to produce a positive PCR diagnostic test result in step (d). In one such preferred embodiment, the PCR diagnostic test performed during the diagnostic pathogen testing step (d) is an RT-PCR diagnostic test performed using the diagnostic testing ready purified pooled RNA containing mixture in determining whether the test yields a positive or negative result.

In a preferred pooled testing method implementation of the present invention, the sample processing step (c) encompasses performing the following substeps: (1) lysing cells in the pooled sample to extract RNA therefrom, (2) binding the pooled RNA lysate to magnetic binding beads, (3) washing the magnetic binding beads to which the pooled RNA is bound to purify the bound pooled RNA, and (4) eluting the purified pooled RNA from the magnetic binding beads. In one such method implementation, in the binding substep (2), the sample containing the RNA lysate is mixed with a proteinase K, a MS2 phage control, a bead-binding solution, and the magnetic binding beads during incubation of the RNA lysate to thereby bind the RNA in the lysate to the magnetic binding beads. In such a method implementation, during the binding substep (2), the sample containing the RNA lysate is mixed with proteinase K, MS2 phage control, bead-binding solution, and magnetic binding beads during the incubation of the RNA lysate thereby binding the RNA in the lysate to the magnetic binding beads. In a preferred test embodiment and testing method implementation, the binding solution includes thiocyanic acid and guanidine, preferably is composed of thiocyanic acid and guanidine, more preferably essentially consists of thiocyanic acid and guanidine, and even more preferably consists of thiocyanic acid and guanidine. Incubation preferably is performed at an incubation temperature that is elevated incubation temperature that is a temperature greater than the temperature at which lysing was performed in substep (1) above.

In another preferred pooled testing method implementation, the diagnostic testing ready purified pooled RNA containing mixture is composed of (i) the purified pooled RNA and (ii) a solution comprised of a buffer, reverse transcriptase, nucleotides (dNTPs), forward primer, reverse primer, a probe, and DNA polymerase, and wherein during the diagnostic testing step (d) the PCR diagnostic test is performed with or using the diagnostic testing ready purified pooled RNA containing mixture. The sample processing step (c) further includes performing the following additional substep of producing a diagnostic testing ready purified pooled RNA containing mixture that includes (i) the purified pooled RNA and (ii) a solution comprised of a buffer, reverse transcriptase, nucleotides (dNTPs), forward primer, reverse primer, a probe, and DNA polymerase, and wherein the diagnostic testing step (d) is, includes or encompasses performing a PCR diagnostic test with or using the diagnostic testing ready purified pooled RNA containing mixture.

In one such preferred pooled testing method implementation, the diagnostic testing ready purified pooled RNA containing mixture is consists essentially of (i) the purified pooled RNA and (ii) a solution comprised of a buffer, reverse transcriptase, nucleotides (dNTPs), forward primer, reverse primer, a probe, and DNA polymerase, and wherein during the diagnostic testing step (d) the PCR diagnostic test is performed with or using the diagnostic testing ready purified pooled RNA containing mixture. The sample processing step (c) further includes performing the following additional substep of producing a diagnostic testing ready purified pooled RNA containing mixture that consists essentially of (i) the purified pooled RNA and (ii) a solution comprised of a buffer, reverse transcriptase, nucleotides (dNTPs), forward primer, reverse primer, a probe, and DNA polymerase, and wherein the diagnostic testing step (d) encompasses and preferably consists essentially of performing a PCR diagnostic test with or using the diagnostic testing ready purified pooled RNA containing mixture.

In another such preferred pooled testing method implementation, the diagnostic testing ready purified pooled RNA containing mixture consists of (i) the purified pooled RNA and (ii) a solution comprised of a buffer, reverse transcriptase, nucleotides (dNTPs), forward primer, reverse primer, a probe, and DNA polymerase, and wherein during the diagnostic testing step (d) the PCR diagnostic test is performed with or using the diagnostic testing ready purified pooled RNA containing mixture. The sample processing step (c) further includes performing the following additional substep of producing a diagnostic testing ready purified pooled RNA containing mixture that consists of (i) the purified pooled RNA and (ii) a solution comprised of a buffer, reverse transcriptase, nucleotides (dNTPs), forward primer, reverse primer, a probe, and DNA polymerase, and wherein the diagnostic testing step (d) encompasses and preferably consists of performing a PCR diagnostic test with or using the diagnostic testing ready purified pooled RNA containing mixture.

Such a PCR diagnostic test preferably also is configured to determine whether a coronavirus, preferably SARS-CoV-2 genetic material, is present in any of the RNA in the diagnostic testing ready purified pooled RNA containing mixture during carrying out step (d). In a preferred embodiment and method implementation, the PCR diagnostic test is configured to determine whether a sufficient amount of SARS-CoV-2 genetic material is present in the RNA in the diagnostic testing ready purified pooled RNA containing mixture to produce a positive PCR diagnostic test result in step (d). In another preferred embodiment, the PCR diagnostic test is configured to determine whether a great enough viral load of SARS-CoV-2 is present to produce a positive PCR diagnostic test result in step (d). In one such preferred embodiment, the PCR diagnostic test performed during the diagnostic pathogen testing step (d) is an RT-PCR diagnostic test performed using the diagnostic testing ready purified pooled RNA containing mixture in determining whether the test yields a positive or negative result.

In one preferred implementation of the pooled pathogen testing method, step (d) of performing a diagnostic test on the pooled sample also is done at the same site or location where the samples are collected from all of the members of the pool. In one such preferred method implementation, at least one and preferably a plurality of (i) the sample or specimen collection, (ii) the pooling of the pooled samples, (iii) the testing of the pooled sample, and (iv) performing pathogen verification testing if the pooled sample tests positive for the pathogen are performed at and preferably onboard a vehicle, such as a mobile testing lab or another type of mobile testing vehicle, preferably at the site or location where the samples are or were taken. In another such preferred implementation, at least a plurality of pairs, i.e., at least three of (i)-(iv) are performed at and preferably onboard a mobile testing vehicle, preferably mobile test lab. In still another such preferred implementation, all of (i)-(iv) are performed at and preferably onboard a mobile testing vehicle, preferably wheeled mobile test lab. In another preferred implementation of the pooled pathogen testing method, the pathogen diagnostic test is performed in step (d) at a testing site located remote from the site or location where the samples were collected from the pool members preferably by being located at least a plurality of miles or kilometers away therefrom.

In one such preferred pooled pathogen testing method implementation the samples from each one of the individuals of the pool that define the pooled sample are placed in the single container or single bag without adding any liquid, e.g., without adding any water, to the single container or single bag and without adding any solution, e.g., without adding any aqueous solution, to the single container or single bag. In such a preferred method implementation, the single container or single bag contains no liquid nor any solution prior to each one of the samples taken from each one of the individuals or members of the pool being added to the single container or single bag to pool together all of the samples from all of the individuals or members of the pool defining the pooled subset sample.

The above-described and illustrated method and system of pooled pathogen testing of the present invention can be used to pathogen test or screen even larger pools having a pool size of greater than 100 individuals, preferably having a pool size of at least 200 individuals, more preferably having a pool size of at least 500 individuals, and even more preferably having a pool size of at least 1,000 individuals. In a preferred embodiment, the present invention is even further directed to such a method and system of pooled pathogen testing of even larger kilo pools having a pool size greater than 1,000 individuals and which preferably is directed to method and system of pooled pathogen testing of even larger pools having a pool size of at least 2,000 individuals, preferably having a pool size of at least 5,000 individuals, more preferably having a pool size of at least 7,500 individuals, and even more preferably having a pool size of at least 10,000 individuals. In another preferred embodiment, the present invention is still further directed to such a method and system of pooled pathogen testing of even larger pools having a pool size greater than 10,000 individuals and which preferably is directed to method and system of pooled pathogen testing of even larger megapools having a pool size of at least 15,000 individuals, preferably having a pool size of at least 20,000 individuals, more preferably having a pool size of at least 50,000 individuals, and even more preferably having a pool size of at least 100,000 individuals.

Other features of the invention will become apparent to those skilled in the art upon review of the following detailed description, claims, and drawings.

### BRIEF DESCRIPTION OF THE DRAWINGS

One or more preferred exemplary embodiments of the invention are illustrated in the accompanying drawings in which:

FIG. 1 is a block diagram illustrating steps of a processed pooled test sample pathogen testing method of the present invention;

FIG. 2 is another block diagram illustrating substeps of a method of obtaining biological samples from individual members of a pool where a first set of samples collected from each pool member are combined together to form the pooled sample that is subsequently pathogen tested and a second set of samples also collected from each pool member are kept separate to be pathogen tested in the event of a positive pooled sample test result to determine which pool members caused the pooled sample to test positive;

FIG. 3A illustrates a perspective view of a concentrating pipette particle bio-concentrator equipped with a pipette tip used to process the pooled sample in at least one step of the pooled sample pathogen testing method of the present invention to produce a processed pooled test sample that provides more repeatably accurate and consistent test results when pathogen tested;

FIG. 3B illustrates a side elevation view of a single use pipette that is usable in the bio-concentrator of FIG. 3A;

FIGS. 4A-4E illustrate a series of steps for a pooling software program for wet media sample pooling configured for and used in carrying out the present invention;

FIGS. 5A-5L illustrate a series of steps for a pooling software program for dry sample pooling configured for and used in carrying out the present invention;

FIG. 6 is a block diagram depicting steps of a pooled pathogen diagnostic testing method of the present invention that includes the steps of collecting a first biological sample from each pool member that is combined into a pooled sample, collecting a second sample from each member that is kept separate from the pooled sample, whereby the first biological samples of all the members that make up the pooled sample is processed into a more refined processed pooled test sample that is pathogen tested, the data obtained from the pathogen testing is analyzed, and the test results are reported to the pool members;

FIG. 7 is a block diagram illustrating steps of the processed pooled sample pathogen testing method whereby the processed pooled test sample pathogen test results are reported to the pool members if negative and each one of the second biological samples of the pool members are individually tested if the processed pooled test sample pathogen test results are positive and the results of the pathogen testing of the individual samples are reported to the corresponding pool members from which the second biological samples were taken;

FIGS. 8A-8B are two halves of a Table 1 referenced in FIG. 8A as Table 1A and in FIG. 8B as Table 1B of preconcentration and concentrated sample values for Theoretical, the Ultra Tip-Std, 0.05 Tip-Std, and Ultra Tip-WW;

FIG. 9 presents Table 2 of concentrated/preconcentrated status for Theoretical, the Ultra Tip-Std, 0.05 Tip-Std, and Ultra Tip-WW;

FIG. 10 presents Table 3 of concentrated values for Theoretical, Ultra Tip-Std, 0.05 Tip-Std, and Ultra Tip-WW;

FIG. 11 presents Graph 1, which is a graph of Ct versus Log Copies/ml;

FIG. 12 presents Graph 2 of Bov Cov Concentration Experiment 4—Ultra Tip, STD Method;

FIG. 13 presents Graph 3 of Bov Cov Concentration Experiment 3—Ultra Tip, WW Method;

FIG. 14 presents Graph 4 of Bov Cov Concentration Experiment 4-0.05 μm Tip, STD Method;

FIG. 15 presents Graph 5 showing InnovaPrep Tip Type and Method Comparison;

FIG. 16 presents Table 4 showing for a starting concentration the percent recovery for the Ultra-STD, 0.05 Std, and Ultra-WW tips;

FIG. 17 presents Table 5 showing run time values for the Ultra-STD, 0.05 Std, and Ultra-WW tips;

FIG. 18 presents Table 6 providing concentrating pipette specifications;

FIG. 19 presents Table 7 providing methods of concentration and effectiveness for Covid Waste Water, Standard Ultra-tip and Standard 0.05 μm tip protocols;

FIG. 20 presents Table 8 providing results of a 50 sample pooling simulation in a 250 of positive in 12.25 mL negative sample matrix;

FIG. 21 presents Table 9 of Starting Sample vs Pooled/Concentrated Sample ORFlab Cts;

FIG. 22 presents Table 10 of Starting Sample vs Pooled/Concentrated Sample N Gene Cts taken from the originally filed provisional in which this application claims priority;

FIG. 23 presents Table 11 providing a listing of components, contents, amounts and storage criteria for a TaqPath™ Covid-19 Combo Kit containing 1000 reactions;

FIG. 24A-24E respectively present separate sections of Table 12A-12E of additional materials not supplied in the TaqPath™ COVID-19 Combo Kit;

FIG. 25 presents Table 13 providing PCR instrumentation and software compatibility;

FIG. 26 presents Table 14 providing quality controls, what the control is used to monitor, and assays used in the control;

FIG. 27 presents Table 15 providing viral particle recovery raw data;

FIG. 28 presents Graph 6 showing the viral particle recovery from swabs providing a comparison of mean Ct values for swabs charged with 20,000 copies of AccuPlex's SARS-CoV-2 standard versus a diluted started;

FIG. 29 presents Graph 7 showing the viral particle recovery from swabs providing a comparison of mean Ct values for swabs charged with 2,000 copies of AccuPlex's SARS-CoV-2 standard versus a diluted started;

FIG. 30 presents Table 16 of Ct values for 20 positive clinical samples—historical individual sample Ct's versus data from five samples pooling method;

FIG. 31 presents Table 17 of ORFlab Ct values for 20 Positive Accuracy Pools;

FIG. 32 presents Table 18 of S Gene Ct Values for Positive Accuracy Pools;

FIG. 33 presents Table 19 of N Gene Ct Values for 20 Positive Accuracy Pools;

FIG. 34 presents Table 20 of Precision ORF lab Ct Data, Precision N Gene Ct Data, and Precision S Gene Ct Data with Ct Values for Precision Pool Samples Compared to Historical Individual Sample Testing;

FIG. 35 presents Graph 8 of Precision—N Gene Ct Data of N Gene Ct Values for Precision Pools Across Four Runs;

FIG. 36 presents Graph 9 of Precision—ORF lab Ct Data of ORFlab Ct Values for Precision Pools Across Four Runs;

FIG. 37 presents Graph 10 of Precision—S Gene Ct Data of S Gene Ct Values for Precision Pools Across Four Runs;

FIG. 38 presents Table 21 showing summary results for 15 clinical evaluation pooled swab samples;

FIG. 39 presents Table 22 listing raw Ct data for pool samples and verification samples for SARS-CoV-2 specific targets;

FIG. 40 presents Table 23 providing a summary of Saline Room Temperature Stability Data for ORF lab for Sample 20210201_00259;

FIG. 41 presents Graph 11 of ORFlab Stability for Sample 20210201_00259;

FIG. 42 presents Table 24 providing a summary of Saline Room Temperature Stability Data for N Gene for Sample 20210201_00259;

FIG. 43 presents Graph 12 of N Gene Stability for Sample 20210201_00259;

FIG. 44 presents Table 25 providing a summary of Saline Room Temperature Stability Data for S Gene for Sample 20210201_00259;

FIG. 45 presents Graph 13 of S Gene Stability for Sample 20210201_00259;

FIG. 46 presents Table 26 providing a summary of VTM Room Temperature Stability Data for ORFlab for Sample X20210111_00110;

FIG. 47 presents Graph 14 of ORFlab Stability for Sample X20210111_00110;

FIG. 48 presents Table 27 providing a summary of VTM Room Temperature Stability Data for N Gene for Sample X20210111-0110;

FIG. 49 presents Graph 15 of N Gene Stability for Sample X20210111_00110;

FIG. 50 presents Table 28 providing a summary of VTM Room Temperature Stability Data for S Gene for Sample X20210111-0110;

FIG. 51 presents Graph 16 of S Gene Stability for Sample X20210111_00110;

FIG. 52 presents Table 29 providing tables summarizing dry swab room temperature stability data for ORFlab, N Gene and S Gene for Sample S20210211_09998;

FIG. 53 presents Graph 17 of Dry Swab Room Temp Stability for Sample S20210211_09998 for ORFlab, N Gene and S Gene at 0, 24, 48 & 72 hours; and

FIG. 54 presents Table 30 providing tables summarizing dry swab room temperature stability data for ORFlab, N Gene and S Gene for Sample S20210210_09999.

Before any embodiments of the invention is explained in detail, it is to be understood that the invention is not limited in its application to the details of construction, implementation, configuration, arrangement, use and operation of the invention as set forth in the following description or illustrated in the drawings. The invention is capable of other embodiments, which can be practiced or carried out in various ways. Also, it is to be understood that the phraseology and terminology employed herein is for the purpose of description and should not be regarded as limiting.

### DETAILED DESCRIPTION OF ONE OR MORE EMBODIMENTS THE INVENTION

Before the present materials and methods are described, it is understood that this invention is not limited to the particular methodology, and materials described, as these may vary. It is also to be understood that the terminology used herein is for the purpose of describing one or more embodiments of the present invention and is not intended to limit the scope of the present invention which will be limited only by the appended claims.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

Currently, diseases, particularly those caused by being infected with a pathogen, such as a virus, bacteria, fungi, yeast, mold, or other microorganism, are tracked by testing people experiencing symptoms or those who may been in close contact with someone that has tested positive for being infected by the associated infectious pathogen. While diagnostic testing is a crucial tool to help track the spread of the infectious disease-causing pathogen, it does not capture the full extent to which the disease nor the disease-causing pathogen may be present within a community. For example, some disease-causing infectious pathogens may not cause symptoms of the associated disease in all people infected with the pathogen, such that infection of the pathogen in asymptomatic carriers may go undetected and unknowingly spread the pathogen to many others.

One method of identifying individuals, even those that are asymptomatic, is by screening individuals by testing pooled biological samples from a group or pool of individuals who may have a particular disease by testing the pooled biological sample for the presence of the infectious disease-causing pathogen. By screening a group or pool individuals to determine if anyone in the batch or pooled samples have a particular disease by testing if anyone who contributed to the batch or pooled samples has been infected with the associated disease-causing infectious pathogen, health officials, employers and others can help prevent the further transmission of disease by actively taking steps to prevent the further spread of the pathogen in those whom testing reveals to be infected with the pathogen.

This can be and typically is done through the use of diagnostic tests that are specifically designed to determine whether individual(s) have been infected with a specific disease-causing infectious pathogen, such as an infectious virus, an infectious bacteria, an infectious fungus, or an infectious mold, and/or a particular disease, such as a viral disease, e.g., COVID-19, yellow fever, Mucor mycosis, tuberculosis, or another type of infectious disease caused by such an infectious pathogen. Pooling samples collected from a group or pool of individuals, however, results in a diluted sample and therefore less viral genetic material. The reduction in viral genetic material present in a pooled sample caused by dilution due to pooling of samples from multiple individuals of the group or pool being screened undesirably increases the likelihood of false negative results due to the increased difficulty of detecting viral material of the infectious pathogen (thus increasing the difficulty of detecting the disease associated with the presence of the infectious pathogen). In order to reduce the number of false negatives, the media or solution containing pathogenic genetic material (from the pathogen) present in the pooled sample may be concentrated through a processing step to increase the probability of detecting the pathogen during subsequent diagnostic testing for the pathogen. While it can depend on the concentration and amount of pathogenic genetic material present in a pooled sample containing at least biological sample from a pathogen-infected person of the group or pool as to whether there is enough genetic material present to prevent a false negative diagnostic test result, it has been learned that it is desirable and even necessary to process a pooled sample containing samples from a group or pool of at least 20 individual pool members in a pooled pathogen diagnostic testing method of the present invention discussed in more detail hereinbelow in order to remove media or solution from the pooled sample while retaining substantially all of the genetic material in the resultant processed pooled test sample upon to ensure prevention of false negatives upon diagnostic pathogen testing being performed thereon.

Testing pooled samples can be very helpful for businesses, companies, schools, other organizations, and events, e.g., entertainment events & conventions, by allowing them to quickly screen people to determine if people are infected with a specific pathogen and also to take actions to help prevent the transmission of that pathogen. Where the presence of a particular pathogen is a clear indication of the presence of a disease associated with the pathogen, a pooled sample pathogen testing method of the prevent invention advantageously enables screening of a relatively large group or even a plurality of relatively large groups of people at a particular location, e.g., business, company, school, organization, or event, including at the same time, by combining one sample taken from each person of the group and combining the samples together into a common pooled sample that is then tested for the presence of the particular pathogen. In at least one preferred embodiment and method, a method of testing pooled samples of the present invention enables quicker screening of groups or pools of people where each group or pool contains at least five people, which can be and preferably is larger than that, preferably containing at least 20 people, more preferably containing at least 25 people, and even more preferably containing at least 30 people, by performing pathogen testing on a pooled sample containing a sample from each person of the such a larger sized group or pool. As discussed in more detail below, it is an advantage of the present invention in that it is scalable so as to enable pooled sample pathogen testing on much larger groups or pools of people, which can be at least as large as 50 people, 100 people, 150 people, 200 people, 250 people, 500 people, and even 1000 people or more.

Where the presence of a particular disease is clearly associated with the presence of a particular pathogen associated with the disease, e.g., causative of the disease, a pooled sample pathogen testing method of the present invention advantageously enables screening of relatively large groups of people at a particular location, e.g., business, company, school, or event, by combining one sample taken from each person of the group together into a pooled sample that is then tested for the presence of the particular disease, preferably by pool testing for the presence of the particular pathogen associated with the disease. In one preferred embodiment and method, a method of diagnostic pathogen testing of pooled samples of the present invention enables quicker screening of a group of people of at least five people that can be larger than that, preferably at least 20 people, more preferably at least 25 people, and even more preferably at least 30 people, by performing pathogen testing on a pooled sample containing one sample from each person of the pool. As with the method of pooled pathogen testing discussed in the preceding paragraph, it is an advantage of the present invention in that it is scalable so as to enable pooled sample pathogen testing on much larger groups or pools of people, which can be at least as large as 50 people, 100 people, 150 people, 200 people, 250 people, 500 people, and even 1000 people or more.

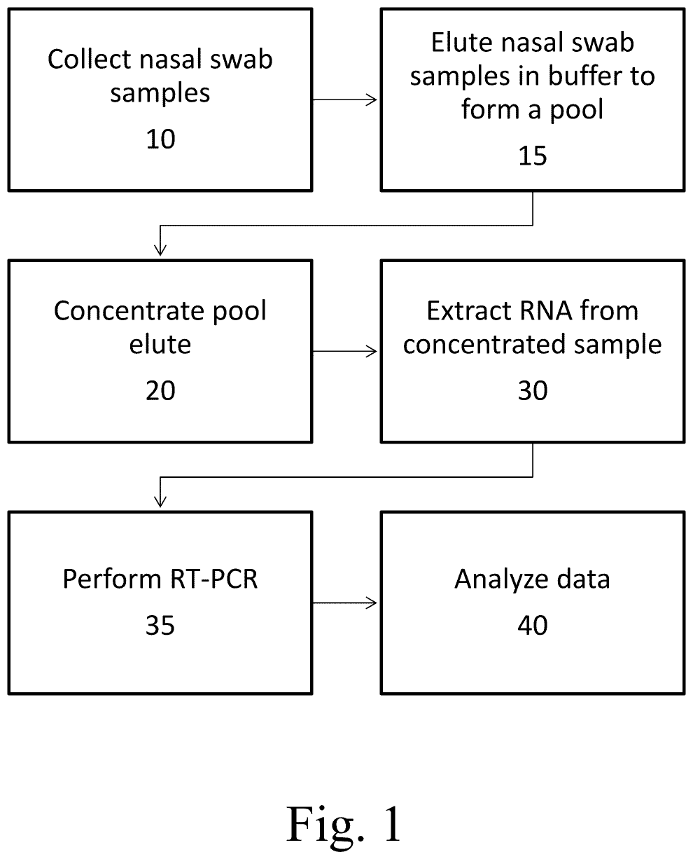

With reference to FIG. 1, in order to test a pool of several people, several dozen people, or even several hundred people using a method of pooled sampling 5 and diagnostic pathogen testing in accordance with the present invention, biological samples are collected, such as by using nasal swabs, from each individual 10 of the pool, eluted and combined to form a batch or pool 15 (“pooled sample”), which can be and preferably is processed before diagnostic pathogen testing by performing substeps of a method of reducing the amount of solution or media in the pooled sample elute discussed in more detail below without substantially reducing the amount of genetic material of the pathogen being screened producing a processed or refined pooled test sample the diagnostic pathogen testing of which advantageously produces a minimum of and preferably substantially completely prevents false negative test results where pathogenic genetic material is present from at least one sample from one person of the pool infected with the pathogen being screened. In one preferred embodiment of the pooled sample diagnostic pathogen testing method of the present invention, one such method of reducing the amount of solution or media pf the pooled sample elute without substantially reducing the amount of genetic material of the pathogen being screened is by processing the pooled sample elute to concentrate the pool elute 20 producing a processed or refined pooled test sample that is a pathogenic genetic material concentrated processed or refined pooled test sample from which RNA of the pathogenic genetic material is extracted in RNA extraction step 25, diagnostically tested, such as by using PCR, preferably RT-PCR, in the specific pathogen diagnostic test step 30, and the data therefrom analyzed in step 35 to determine if anyone in the pool or batch has been infected with the specified pathogen being screened. In at least one preferred implementation of a pooled sample pathogen testing method of the present invention, the method includes and requires the step of processing the pooled samples, i.e., pooled sample elute, to reduce the amount or volume of solution or media without substantially reducing the amount of genetic material of the pathogen being screened to produce a such processed or refined pooled test sample that can be a pathogenic genetic material concentrated processed or refined pooled test sample produced from carrying out step 20 of the pooled sample diagnostic pathogen testing method depicted in the diagram, e.g., flowchart, of FIG. 1.

With more general reference to FIG. 1, in order to test several people through a method of pooled sampling 5 in accordance with the present invention, biological samples are collected, such as by using nasal swabs, from each individual 10 of a pool of a relatively large group of people, whereas the biological samples may be eluted and combined to form a batch or pool 15, concentrate the pool elute (if needed) 20, extract RNA from the sample/concentrated sample 25, perform a diagnostic test to test for a specific disease 30, preferably by testing for a particular infectious pathogen causal of the disease, and analyze the resultant test data 35 to determine if anyone in the pool has the particular disease being screened for by the test preferably by determining if anyone in the pool has been infected with the specified pathogen that causes the disease. In at least one preferred implementation of a pooled sample pathogen testing method of the present invention, the method includes and requires the step of concentrating the elute of the pooled sample produced from combining or convolving the samples from all of the individuals of the pool, before RNA extraction, testing and analyzing the test data.

By testing everyone, and not just those who are experiencing symptoms of the pathogen-caused disease or those who have been in close contact with someone who has tested positive for the pathogen causing the disease, the method of pooled sample testing 5 of the present invention depicted by FIG. 1 advantageously is able to capture a more complete disease profile of a community. For example, pooled sample testing 5 is able to determine if an asymptomatic person has been infected with the pathogen and steps can be taken to prevent transmission of the disease preferably by preventing spreading of the pathogen. Companies, schools, organizations and/or events may collect biological samples periodically from respective groups of employees, students, faculty, enrollees, attendees, etc., pool those samples, and test each pooled sample to determine if anyone in the pool has been infected with the particular pathogen or specified disease being screened for by the test. Where a pooled sample is tested and produces a negative result, it advantageously eliminates the need for individual testing of each person, individual or member of the pool such that pooling samples and testing such pooled samples, time, resources, and money can be saved.

In addition to testing pooled samples 5 in a “community” (e.g., school or company), another form of testing “pooled” samples may be using wastewater to screen for disease or detect early variants of a disease preferably by the test being configured to detect the presence in the pooled sample of genetic material of the specific type of pathogen that causes the disease. While wastewater monitoring does not replace traditional pathogen or disease testing, wastewater monitoring does assist in providing a better understanding of disease activity, including by providing a better understanding of the spread and rate of spread of its associated pathogen, which can be used to help slow the spread of disease in a community preferably by reducing pathogen transmissibility. Some diseases or pathogens may be detected in feces shortly after the disease has infected a person. For example, evidence suggests that SARS-CoV-2, otherwise known as COVID-19, is a disease-causing pathogen that can infect various tissues of a person, specifically those expressing the angiotensin-converting enzyme (ACE2) protein, which includes the gastrointestinal lining. Analysis of stool from both symptomatic and asymptomatic patients shows high prevalence of viral shedding (30-90% of infected individuals) accompanied by high individual viral shed rates (up to 7.5 log 10 per gram of feces). COVID-19, therefore, may be detected in feces even before people experience symptoms or are asymptomatic through the detection of the SARS-CoV-2 viral pathogen that causes COVID-19 during diagnostic testing specifically designed or configured to detect SARS-CoV-2.

Evidence of high viral loads in stool substantiates the use of wastewater as a potential mechanism to monitor population infection rates. Wastewater monitoring may therefore be applied to act as an early warning tool to better assess, predict and contain viral outbreaks at the community level. The additional information can also help local communities intervene with disease mitigation strategies and demine how well protective measures (masks, lockdowns, or quarantine) are working.

Typically, in order to conduct wastewater disease screening, large liquid volume (e.g., greater than 1 L) must be concentrated through two stages—a primary concentration and a secondary concentration. Challenges for concentrating large liquid volumes in multiple steps include loss of viral titer during each step of concentration and significantly increasing sample processing time. As discussed below, the same method of concentrating pooled biological samples, disclosed hereinafter, may be applied to wastewater thereby allowing wastewater to be screened for pathogens (e.g., COVID-19) and act as an earlier detection system or provide other information to communities.

In order to screen for disease in either individuals or a general population, biological samples or specimens must be collected from individuals, as seen in FIG. 1. Once the specimens have been collected, the specimens are then processed. Depending on the number of specimens in a batch, or pool, the pooled samples or eluate may be concentrated to increase the concentration of genetic material in the eluate. The pooled eluate then has RNA extracted and purified, before RT-PCR is used to determine whether the pooled samples contain the genetic material of the specified pathogen.

### I. Collecting Biological Samples

In order to collect biological samples or specimens from test subjects, or individuals that desire or need to be tested, biological samples or specimens may be obtained from saliva, nasopharyngeal swabs, nasopharyngeal aspirate, nasal swabs, and mid-turbinate swabs, and bronchoalveolar lavage samples. In one embodiment, a sterile nasal swab may be used. In yet another embodiment, the swabs used to sample the anterior nares may be oropharyngeal swabs. The nasal swab includes a tip, formed from polyester, and a handle. In order to collect or obtain a first biological sample, the tip of the nasal swab may be inserted deep enough into the nasal cavity, or anterior nares, of a first nostril so that the tip of the nasal swab is no longer visible. The nasal swab may then be rotated around the interior or inner surface of the nasal cavity at least one, preferably at least thrice, and more preferably at least five times. The tip of the nasal swab can then be removed from the first nostril and then inserted deep enough into the second nostril so that the tip of the nasal swab is not visible. The tip of the nasal swab can then be rotated around the inner or interior surface of the second nostril at least once, preferably at least thrice, and more preferably at least five times.

Once a first biological sample has been obtained by swabbing both sides of the nasal passageway, the nasal swab may be placed within a sterile tube containing media, and vigorously swirled or rotated within the media. The sterile tube is preferably a conical tube having a lid or cap, and preferably a 15 mL conical tube. The media within the tube may be saline. There may further be 2-4 mL of saline or media in the tube, but the amount of media may vary in other embodiments. The nasal swab may be swirled within the media for five to thirty seconds, preferably at least five seconds, and more preferably at least ten seconds. Once the nasal swab has been swirled within the media and the biological sample released into the media, the swab may be removed from the media and discarded. The swab is preferably slowly removed from the media so that the media or liquid is removed or squeezed out of the tip of the nasal swab so that the media drips or flows back into the tube. The first nasal swab may then be discarded. The cap may then be tightened, snapped shut, twisted, or otherwise fastened so that the tube and cap engage each other and the media, containing the biological sample, is secured within the tube.

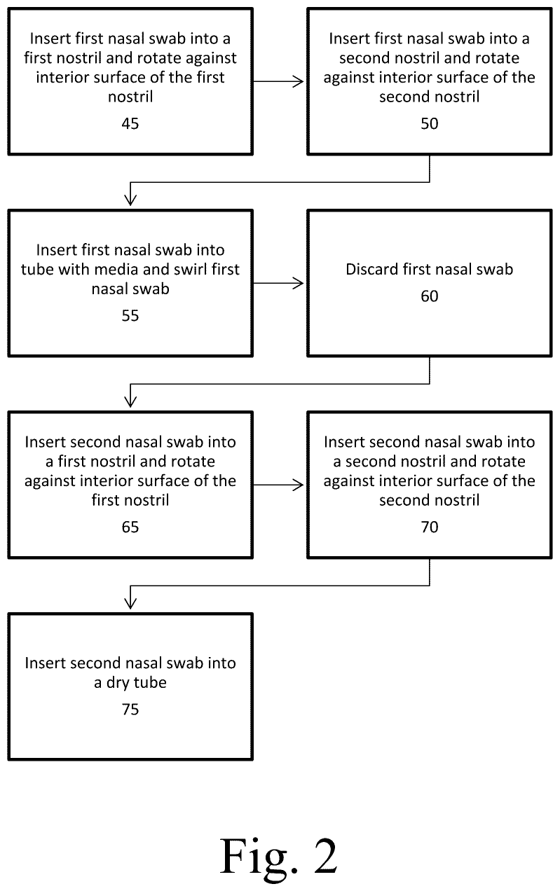

In alternative embodiments and as illustrated in FIG. 2, two biological samples may be collected from each individual so that the first biological sample may be combined with other biological samples to create a pool of samples, and the second biological sample may be reserved for individual testing as a follow-up or secondary testing if the test with the pooled samples results in a positive result for a specified disease, as will be explained hereinafter. The first and second biological samples may be collected through the same method, and as described above. In other words, a first nasal swab may be inserted up both a first nostril 45 and before being inserted up a second nostril 50 and rotated along the inner surface for each nostril to obtain a first biological sample. The second biological sample may be collected the same way as the first biological sample—inserted up a first nostril and rotated 65 before inserted up a second nostril and rotated 70. The first biological sample may be swirled in media 55 before being discarded 60, as described above. For the second biological sample, however, the second nasal swab may be placed within a sterile, DNase/RNase-free, dry or an empty container or tube 75, where the container or tube is preferably a conical tube. The conical tube may be a 15 mL tube, a 50 mL tube, or any other container or bag of sufficient size to contain at least one swab. The tubes may further be labeled with the donor's name, date of birth and collection date.

While the process of collecting biological samples or specimens may be identical or substantially similar and does not change according to the number of samples within a batch or pool, the process of preparing the samples may change as will be described hereinafter.

### II. Sample Processing for Up to Five Samples

For example, when pooling approximately fifteen or less samples, preferably ten or less samples, or more preferably five or less samples, the media containing the biological samples may all be combined and placed within a single well of a 96 deepwell plate. In embodiments where each biological sample has 80 μL of media added, each well will contain 400 μL of media when combining or pooling five samples. Thus, up to four hundred and seventy samples may be placed within a 96 deepwell plate and analyzed. Once the biological samples have been loaded on the 96 deepwell plate, additional reagents or materials necessary for RNA extraction and purification may be added to the wells of the 96 deepwell plate, as will be discussed hereinafter.

### III. Sample Processing for Up to Twenty-Five Samples

As discussed previously, a first biological sample and a second biological sample may be taken from each individual 10 where the first biological sample is swirled within approximately 2-4 mL of media and the second biological sample is placed or stored within a sterile, labeled, RNase-free, dry tube. Preferably, the second biological sample may be placed within a 50 mL conical tube so that additional biological samples may also be placed within the 50 mL tube until there are thirty or less biological samples, and preferably twenty-five biological samples or less within the tube. 4-8 mL of media is then added to elute the samples, preferably 5-7 mL of media is added to elute the samples, more preferably 6 mL of media is added to elute the samples. Again, the media may be saline or any other suitable substance to transport viral material.

Alternatively, instead of the second biological samples taken from individuals being placed within an empty tube, the tube may already contain the desired amount of media (i.e., 4-8 mL of media) and each swab may be fully submerged in the liquid media and vigorously swirled in the media for at least ten seconds. As much liquid as possible is preferably released from the second swabs along the sides of the conical tube, before the swabs are removed from the liquid and discarded. The tube containing the biological samples and media may then be vortexed for at least thirty seconds to release all the particulates from the swabs into the media. A transfer pipet or serological pipet may be used to remove all liquid media into a clean 15 mL conical tube to prepare for RNA extraction and purification.

### IV. Sample Processing for Twenty-Five to One Hundred Samples

Again, as discussed previously, a first biological sample and a second biological sample may be collected from an individual 10. The first biological sample may be combined with other samples to create a pool of samples for testing. The other or second biological sample may be saved as a biological specimen for future tests if additional testing is needed in the future (e.g., the second sample may be used and tested to determine if an individual is positive for a specific disease if the pooled samples receive a positive result for a specific disease).

In order to elute twenty-five or more samples, preferably between twenty-five and five hundred samples, and more preferably between fifty and a hundred samples, into a sample pool, the first biological samples (i.e., the first swab) from each individual may be placed into a stomacher bag with the appropriate volume of media for pool size. The media may include sterile saline and 0.05% Tween-20. For example, twenty-five samples may be eluted in approximately 4-8 mL of saline or other media and preferably with 6 mL of saline or other media. Fifty samples may be eluted in approximately 10-30 mL of saline or other media, preferably with 25 mL of saline or other media, and more preferably 12.5 mL of saline or other media. A hundred samples may be eluted in approximately 20-60 mL of saline or other media, preferably with 50 mL of saline or other media, and more preferably 25 mL of saline or other media.

The stomacher bag is then folded to make a “pocket” with the swabs and media within the pocket. The fold may then be secured through tape, clips, or other methods so that the pocket may be maintained. The stomacher bag is then placed onto a stomacher, on “high” for approximately 60 seconds on one side and 30 seconds on the opposite side of the stomacher bag. In embodiments where two or more stomacher bags are eluted at the same time, the bags are preferably positioned so that they slightly offset. Within a Class II biosafety cabinet, the eluate is transferred into 50 mL conical tubes and ready to be concentrated if so required.

Sample pools involving twenty-five or less samples eluted in 6 mL of media does not require the eluates to be concentrated. The resulting eluates from sample pools having greater than twenty-five samples, preferably less than fifty samples eluted with at least 25 mL of media, and less than a hundred samples eluted with at least 50 mL of media, are preferably concentrated into a 0.7-1 mL elute before RNA is extracted from the resulting eluate. In order to concentrate the eluate for sample pools preferably greater than twenty-five samples, an InnovaPrep Concentrating Pipette Select 80 particle or nanoparticle bio-concentrator or other automated bio-concentrator system or method of concentrating viral genetic material from liquid may be used to exponentially improve the limit of detection for trace pathogens through PCR, rapid antigen tests, or other methods.

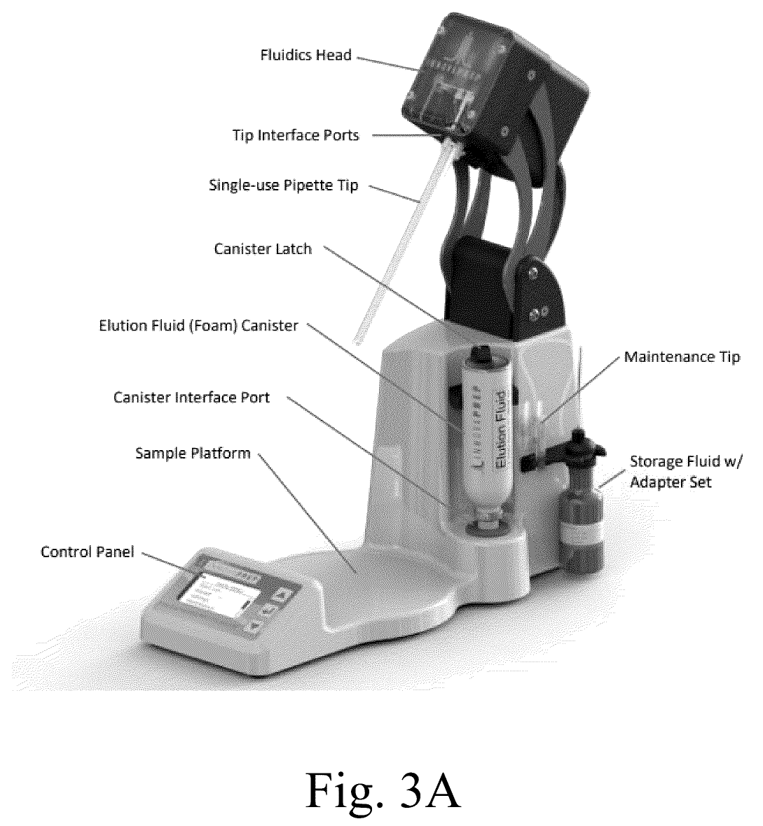

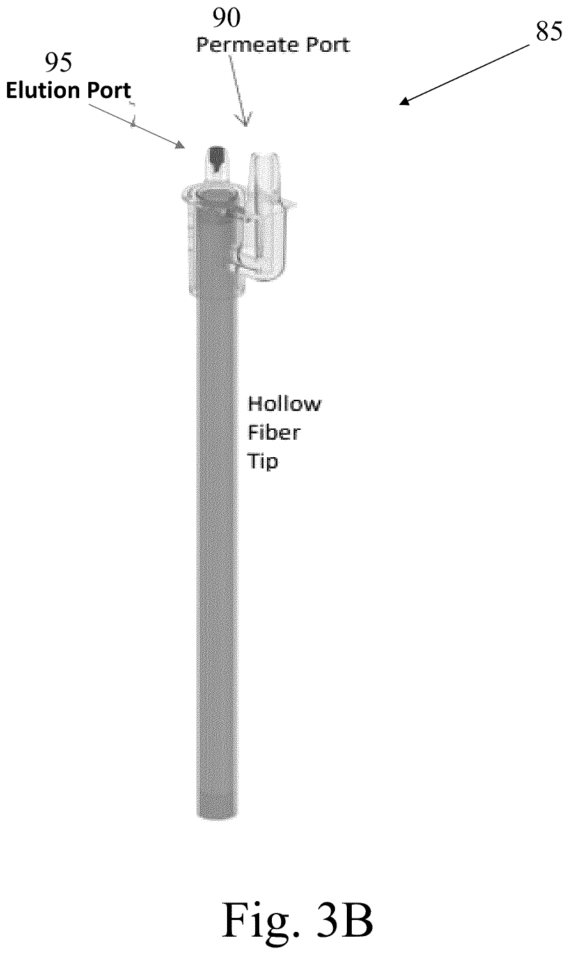

Referring to FIGS. 3A-3B, in embodiments, where an InnovaPrep Concentrating Pipette Select 80 particle bio-concentrator is used, the instrument may be used as a method of filtering and concentrating up to five liters of liquid at 200 mL per minute through its high-flow single-use pipette tips 85. The filtration through the single-use pipette tips 85 is preferably dead-end filtration and may include either vacuuming or pumping to force the liquid or media through the single-use pipette tips 85. In one embodiment, the InnovaPrep Concentrating Pipette Select 80 particle bio-concentrator includes a main pump that draws a vacuum on a Permeate Port 90. The main pump draws the air inside the filter housing out and the liquid sample is pulled up through a Sample Port.

The liquid sample is drawn through the single-use pipette tip, where the internal filter(s) of the pipette tips capture particles (e.g., viral particles) based on the pore size of the membrane within the single-use pipette tips. The internal filter(s) are made with either a flat membrane or a bundle of hollow fiber membranes. As the sample is drawn through the filter, particles larger than the chosen membrane pore size are captured on surface of the porous membrane filter. On the other hand, liquids, dissolved solids and particles smaller than the chosen pore size pass through the filter and into the permeate. Once the sample container is empty, air is drawn up behind the liquid and the membrane “locks up,” leaving only the target particles on the membrane and ending the run. Once the run is over, the particles may be recovered from the membrane by eluting the concentrated particles through the sample port of the filter tip in a preset volume (e.g., 0.15-1 mL) of buffer.

Contamination is prevented or reduced between samples as the pipette tips are single use only. The liquid containing the biological samples are therefore only in contact with the single-use pipette tips thereby preventing or reducing contamination between samples. The single-use pipette tips 85 are single use only because filters within the pipette tips are hydrophilic, meaning that air is allowed to flow through only when the membrane is dry. Once the filter has been wetted, the surface tension of the liquid trapped in the pores of the membrane prevents air (or any other gas) from passing through the membrane. Thus, the single-use pipette tips 85 cannot be re-used since air trapped inside the housing cannot be pulled through the filter prior to processing a second sample.

While a water, buffer, or other liquid may be used to wash and recover the particles off the membrane, the Wet Foam Elution process is preferable because it is more efficient. During the Wet Foam Elution process, the Permeate Port valve 90 closes, and the elution valve opens, allowing the foam to enter an Elution Port 95. The foam travels tangentially down the surface of the membrane as it washes the particles from the surface. The concentrated sample is then pushed out of the sample port into a cuvette or other container where the foam quickly breaks back down into a small liquid volume.

The Wet Foam Elution™ process requires very specific high-quality foam in order to be effective. The elution fluid is composed of water, a low concentration surfactant (usually less than 0.1%), and a pH buffer. This solution is carbonated with carbon dioxide (CO2) gas. During the elution process, the fluid passes through a valve to a low-pressure environment, causing the dissolved CO2 to expand and come out of solution to form microbubbles. These microbubbles increase the volume of the fluid six-fold or more. An additional benefit of Wet Foam Elution is the clean buffer exchange. In many situations, the starting sample matrix is not the most desirable for the chosen analysis method. Wet Foam Elution allows the user to select the fluid that the particles will be suspended in after concentration, which reduces inhibition and maximizes the chances of detection.

Furthermore, the Wet Foam Elution process is more efficient than eluting with liquid because of the volume of water (as compared to gas), the increased viscosity of foam, bubble dynamics, and exfoliating action. Generally speaking, when a filter is rinsed with water, most of the liquid volume is used to fill the dead space inside the filter housing or inner bore of the membrane for hollow fiber tips 85. Thus, only a small portion of the fluid is actually in contact with the filter surface. This can be minimized to an extent by reducing the cross-sectional area of the fluid path across the filter, but a large portion of the liquid is still underutilized. Foam, however, is 80-90% gas, which fills the empty space without contributing to the final sample volume. Furthermore, liquid has a tendency toward “channeling” when flowing across a surface such that there is an area of high flow in the center of the fluid path while the portion of flow in contact with the filter surface is much slower. The higher viscosity of foam prevents channeling and creates a more uniform flow across the filter surface. As for bubble dynamics, the microbubbles in the foam behave as deformable solids. As they travel across the surface of the filter they move as a rigid body with a narrow lubricating layer, effectively squeegeeing the particles off of the surface. The turbulence and energy produced when micro-bubbles in the foam impact against each other and burst also helps lift particles that are adhering to the membrane.

The run and elution may further be customized through adjusting elution fluid valve open time, pulse count, foam factor, flow start, flow end, flow min start, ext delay sec, pump power, and ext pump delay. The final volume of the sample is controlled primarily by the elution fluid valve open time. The longer the open time and the more pulses performed, the larger the elution volume. Valve Open milliseconds controls the length of time that the elution valve is open, per pulse, in milliseconds and ranges from 25-999 milliseconds (ms). Pulse Count is the number of cycles (ranging from 1-25 ms) that the elution valve will open and close. Multiple pulses are usually used when larger (>200 μL) final volumes are desired. For example, in some situations, recovery may be more efficient if two 100-ms pulses are performed rather than one 200-ms pulse while maintaining the same final elution volume.

Foam Factor sets the release frequency of the foam valve during elution, ranging from 0-100 ms, and may result in improved recovery with certain matrices. If set to 10, the foam valve will power on for 5 ms, then off for 5 ms, repeatedly for the duration of the valve on time. If set to 5, the foam valve will power on for 2.5 ms, then off for 2.5 ms, repeatedly for the duration of the valve on time.

Flow Start, which can be set between 0 and 5 seconds, determines the flow sensor sensitivity needed to establish liquid “flow” on the instrument. As the liquid is processed, a sensor detects the liquid. This sensor is used along with a time stamp “Flow Start” to establish that there is flow. The lower the number the less liquid required to establish flow. If there is no established flow the unit will run until the Flow Min Start time is reached. Flow Start may need to be adjusted when working with very small sample volumes (i.e., <10 mL). Flow Min Start, which may be set between 1 second and 1 minute, determines the length of time that the unit will run without liquid flow before the unit times out. The lower the value the quicker the unit will timeout if it does not see flow at the start of a run. Viscous fluids and lower flow rate pipette tips, such as the ultrafilter pipette tips, may require a higher set point. Flow End determines the flow sensor sensitivity needed to establish “no flow.” Once “no flow” is established the unit will shut down. The lower the number the quicker the unit will shut down after the fluid is processed. Pipette types (e.g., Ultrafilter CPTs) and sample matrices that run at low flow rates may require a lower setting to ensure that the instrument shuts down at the end of the sample concentration run. Settings between 0-20 seconds may be set, and a setting of 0 seconds may be used to allow the user to manually end the sample run using the menu.

Ext Delay Sec sets the delay time, ranging from 0.1-10 seconds, between the vacuum relief and the foam valve opening. The delay allows the pressure on the permeate side of the membrane in the pipette tip to achieve equilibrium. Ideally, changes to the Valve Open time and Pulse count should be considered first. Pump Power sets the pump duty cycle, where the setting ranges from 25% to 100% and a setting of 50 is 50% power. A lower power setting may increase efficiency and reduce fouling with some matrices; however, the initial process flow rate will be reduced. Ext Pump Delay, ranging from 0.1-10 seconds, sets the delay after the foam valve open time before the permeate pump is turned on to remove residual fluid. Like Ext Delay Sec, changes to the Valve Open time and Pulse count should be considered first.

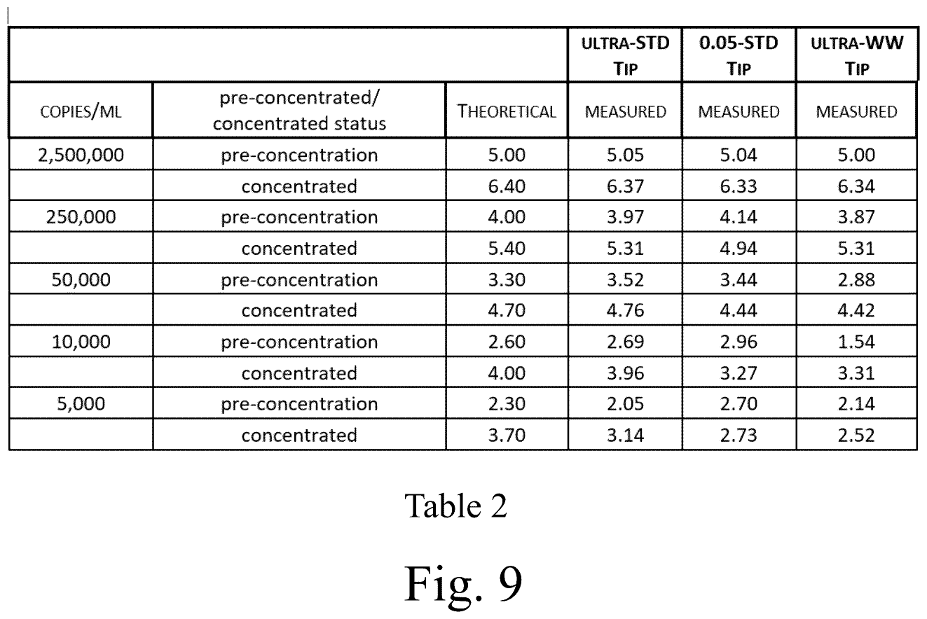

Another method of adjusting the concentration of the media is through the selection of the pipette tip 85. For example, a standard wastewater pipette tip, an 0.05 μm pore size tip, or an ultra-filtration tip may be used to adjust the recovery of particles during concentration of the media. While the standard pipette tip may be used for concentration, use of either the 0.05 μm or ultra-filtration tips results in a better recovery of virial genetic material than standard tips (assuming nothing else is changed). As shown in Table 2 of FIG. 9, both the 0.05 μm and ultra-filtration tip have filters formed from polysulfone and have a membrane surface area of 98 cm 2. While the concentrated sample volume for both tips ranges from 150-1000 μL, the ultra-filtration tip can only concentrate media volumes up to 500 ml. The 0.05 μm tip, on the other hand, can handle up to 3 L of media. The ultra-filtration tip, however, has a faster processing rate (50 ml/min) than the 0.05 μm pipette tip (up to 90 ml/min).

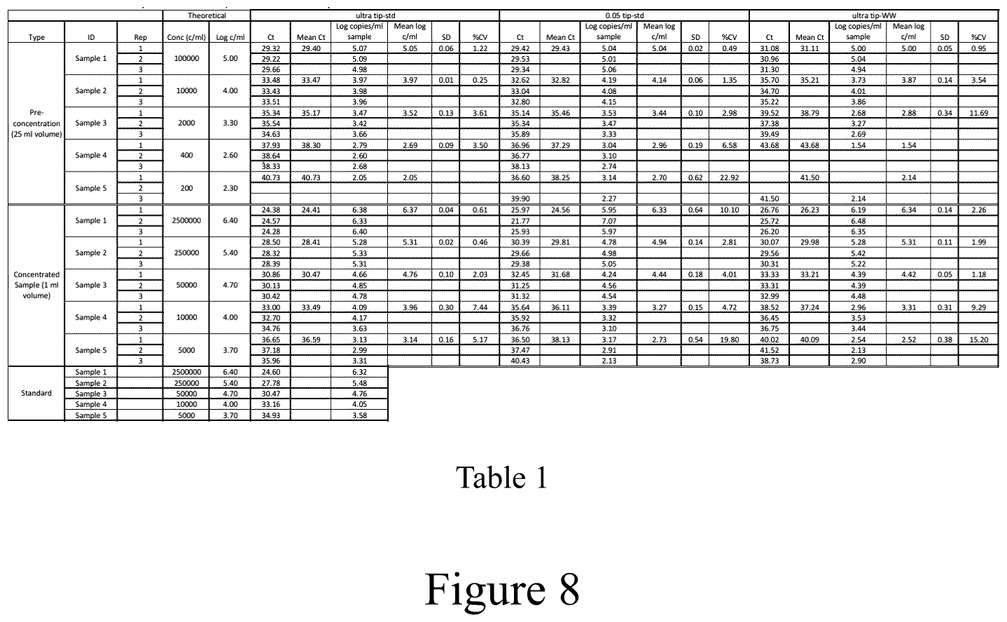

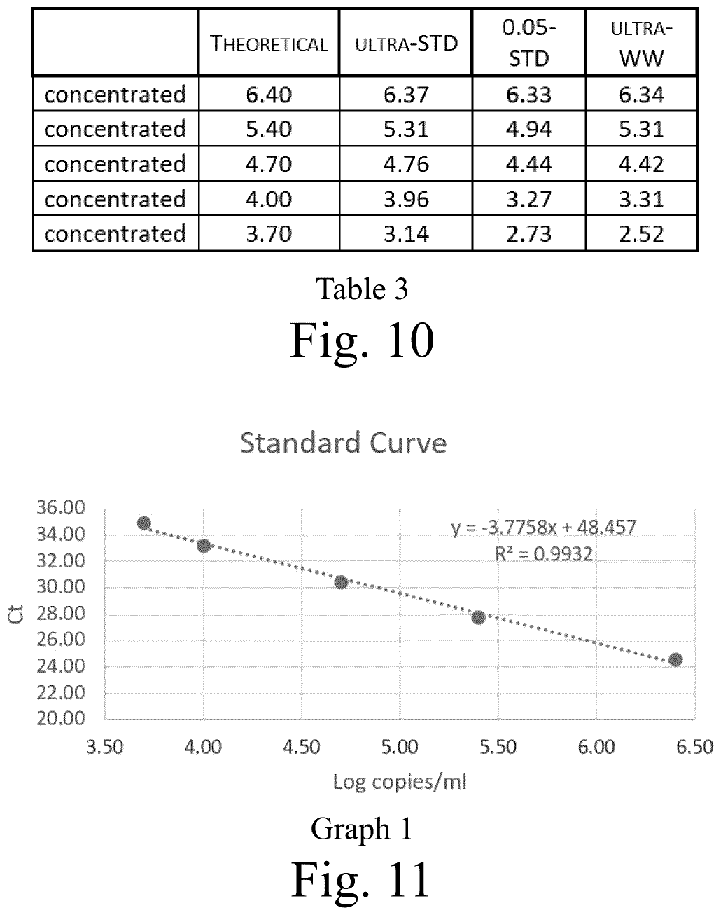

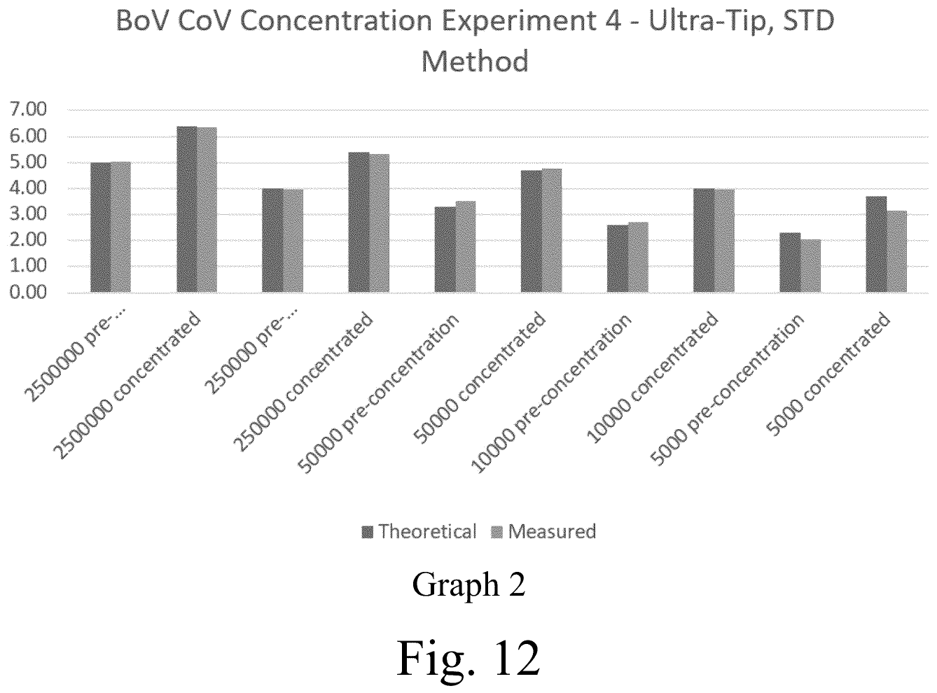

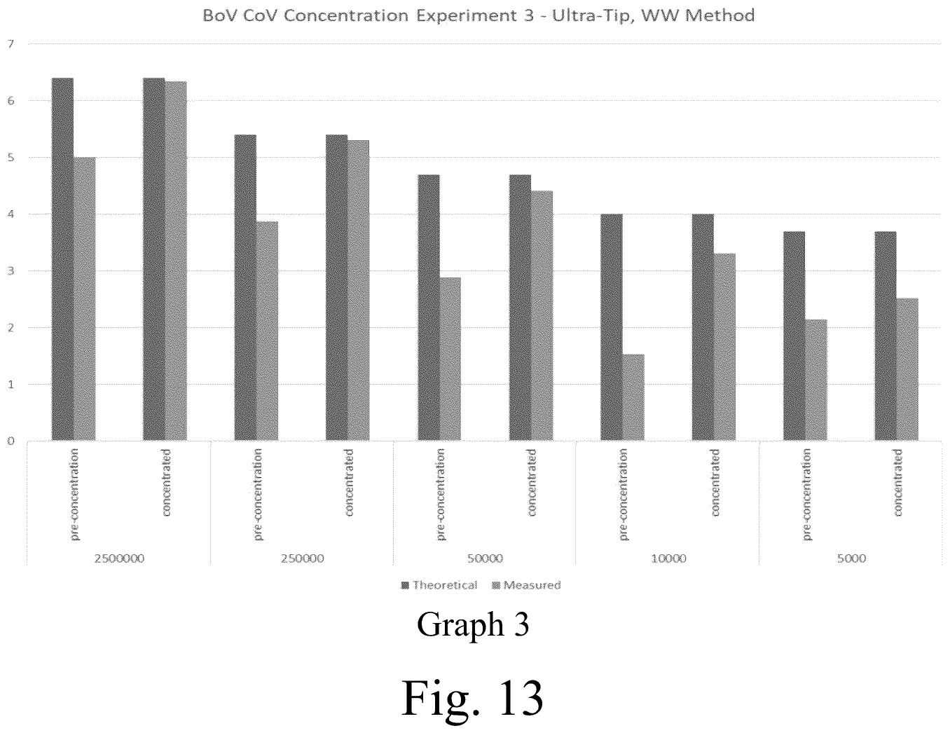

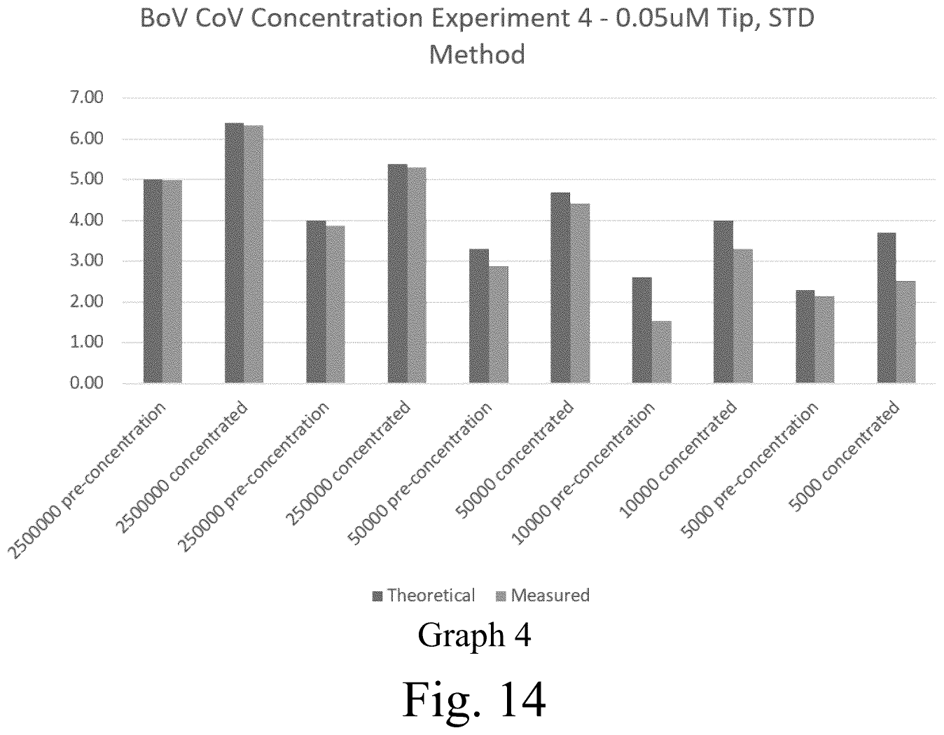

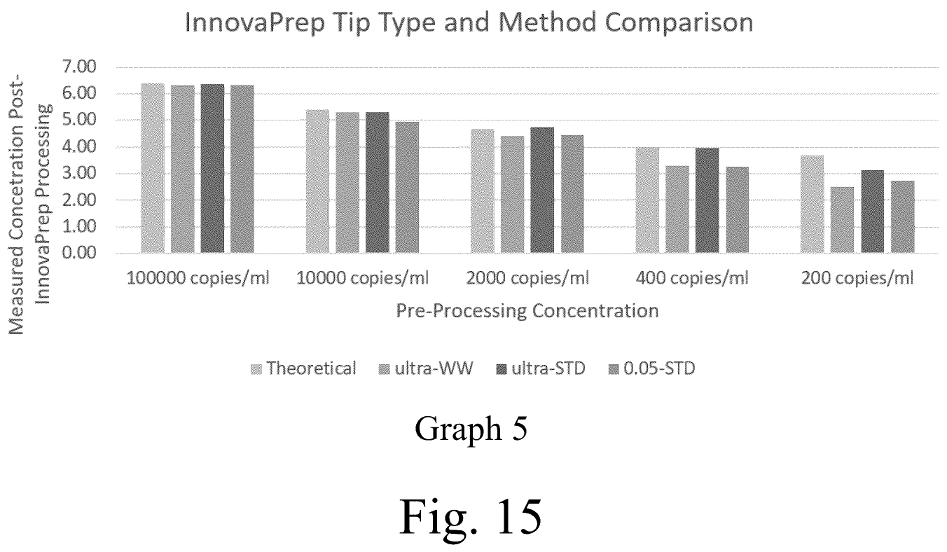

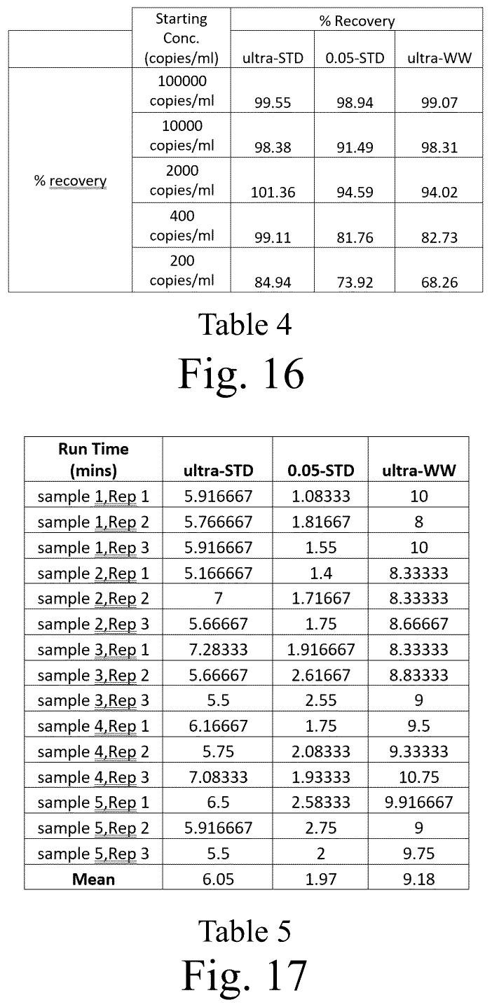

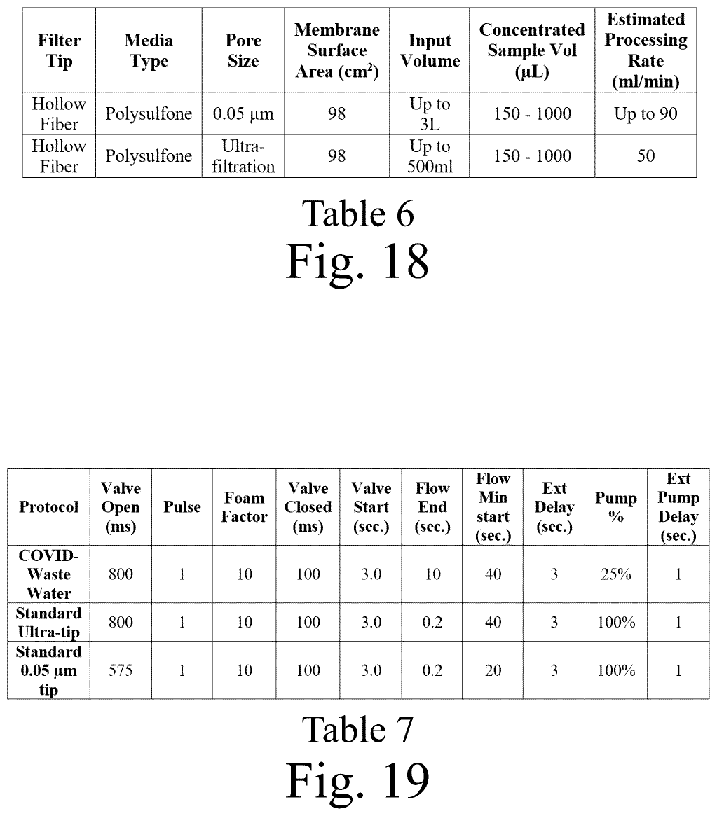

The tables and graphs of FIGS. 8A & 8B (Table 1A & Table 1B), FIG. 9 (Table 2), FIG. 10 (Table 3), FIG. 11 (Graph 1), FIG. 12 (Graph 2), FIG. 13 (Graph 3), FIG. 14 (Graph 4), FIG. 15 (Graph 5), FIG. 16 (Table 4), and FIG. 17 (Table 5) provide a comparison of run time versus level of recovery using the particle bio-concentrator tips identified and discussed above and below herein.

Thus, with reference to FIGS. 8A-17, the standard and ultra-filtration tip protocols likely take approximately six minutes to concentrate a sample, per 25 ml input. The wastewater method, on the other hand, takes approximately ten minutes to concentrate a 25 ml sample input. The difference in concentration time between the wastewater and standard method is likely the wastewater method uses a pump percentage of 25%, therefore increasing the time needed to pump the solution through the ultra-filtration tip. The standard method uses a pump % of 100%. The difference in pump percentage did not significantly impact recovery in these experiments.

While the 0.05 um tip is significantly faster (i.e., under two minutes), there is significant loss in recovery at about 400 copies/ml. The difference in recovery between the tips may be related to the time it takes for the sample to flow through the tip fibers. The increased pore size of the 0.05 μm fibers allows the liquid to flow through the tip much more quickly (approximately two minutes vs. approximately six minutes). Viral particles may be lost to the permeate with the 0.05 μm tips.

Additionally, because the TaqPath assay requires approximately 250 copies/ml, the ultra-filtration tip is preferred for a 50-swab pool in 12.5 ml of buffer and 100 swab pool in 25 ml of buffer due to its level of recovery and time per sample. While both the 0.05 um and the ultra-filtration tip types are suitable for viral concentration, however the ultra-filtration is preferred. The type of tip preference may differ due to the matrix composition, sample retention time within the concentrating tip, and sample temperature. The sample temperature may alter time needed for concentration in that lower temperature samples (straight from 4° C. storage) tend to take longer to process and demonstrate slightly lower recovery than samples brought to room temperature prior to processing.

As seen in Table 7 of FIG. 19, possible adjustments to the InnovaPrep Concentrating Pipette Select bio-concentrator to aid in recovery of viral particles include selecting a COVID-Waste Water tip and adjusting the settings on the InnovaPrep Concentrating Pipette Select bio-concentrator so that the Valve Open is for 800 milliseconds, Pulse is set on 1, Foam Factor is set on 10, Valve Closed is set for 100 milliseconds, Valve Start is set for 3 seconds, Flow End is set for 10 seconds, Flow Min start is set for 40 seconds, Ext Delay for 3 seconds, Pump is set at 25%, and Ext Pump Delay at 1 second. Alternatively, an ultra-tip pipette tip may be selected and the InnovaPrep Concentrating Pipette Select's settings adjusted so that the Valve Open is for 800 milliseconds, Pulse is set on 1, Foam Factor is set on 10, Valve Closed is set for 100 milliseconds, Valve Start is set for 3 seconds, Flow End is set for 0.2 seconds, Flow Min start is set for 40 seconds, Ext Delay for 3 seconds, Pump is set at 25%, and Ext Pump Delay at 1 second. Yet another possible selection and settings may include a 0.05 μm pipette tip where the Valve Open is for 575 milliseconds, Pulse is set on 1, Foam Factor is set on 10, Valve Closed is set for 100 milliseconds, Valve Start is set for 3 seconds, Flow End is set for 10 seconds, Flow Min start is set for 20 seconds, Ext Delay for 3 seconds, Pump is set at 25%, and Ext Pump Delay at 1 second.

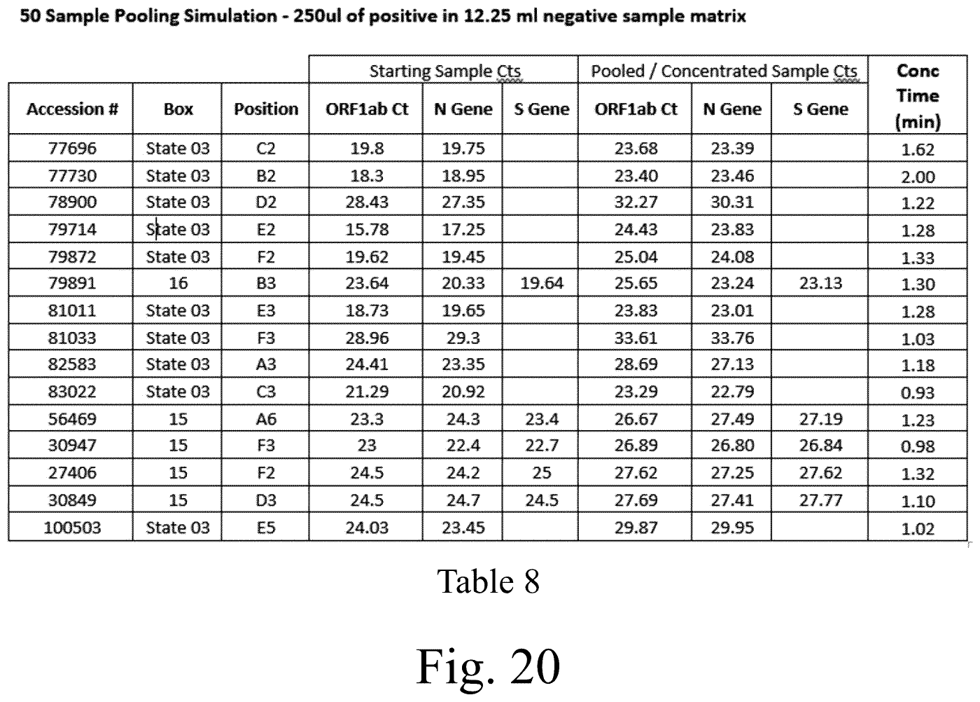

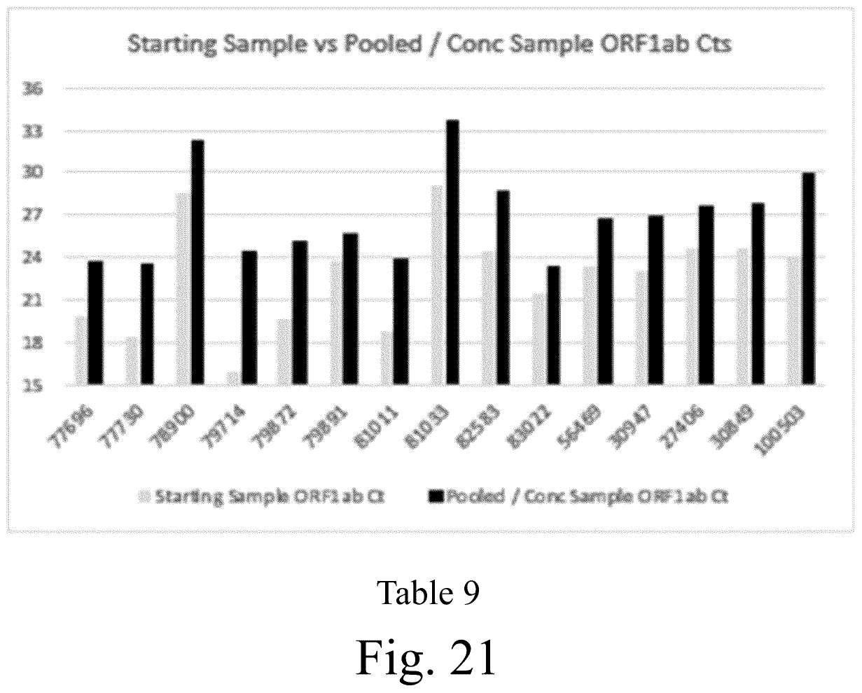

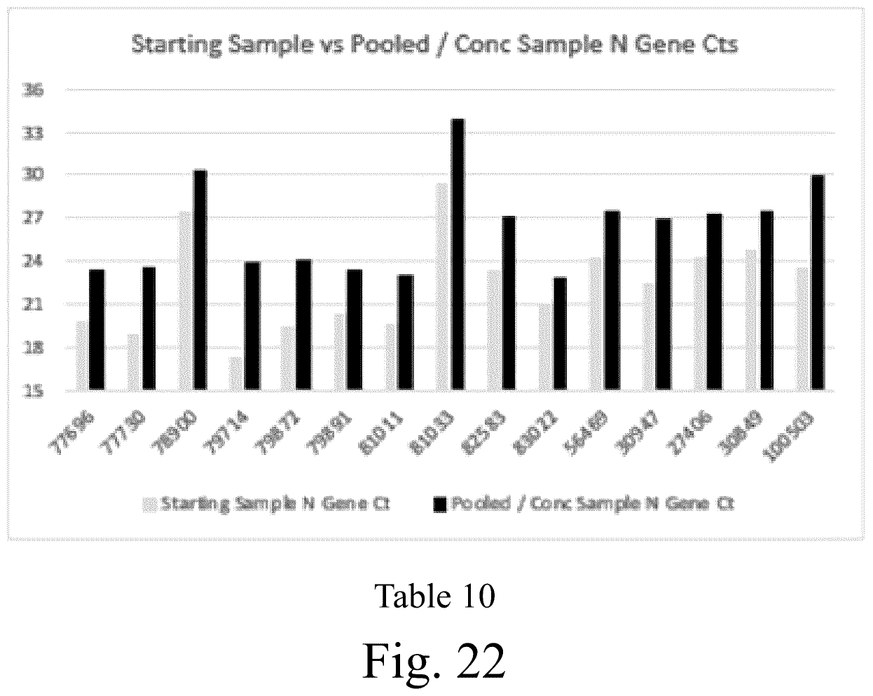

As seen in Table 8 of FIG. 20, which provides the results of a 50 Sample Pooling Simulation—250 μL of positive in 12.25 ml negative sample matrix, and associated graphs, the InnovaPrep CP Select particle bio-concentrator system increases the cycle threshold values or Cts as compared to the starting samples and concentrated samples. The total sample input volume is 12.5 ml resulting from fifty samples each consisting of 250 μL. On average, it took approximately one minute and fifteen seconds to concentrate the sample, where the resulting concentrated volume is approximately 700-1000 μL. The extraction input volume is 200 while the extraction elute volume is 50 μL.

Once the liquid media, containing the pooled biological samples, has been concentrated, the concentrated eluate may then be used for RNA extraction. In one embodiment, the concentrated eluate may have been eluted with 200 and the entire 200 μL may be placed within a single well of a 96-well plate, and preferably a 96 deep-well plate, for RNA extraction.

### V. RNA Extraction and RT-PCR

Reagents may further be added to the wells for RNA extraction and RT-PCR (reverse-transcription polymerase chain reaction). The reagents necessary to isolate and purify the RNA extracted from the samples and to perform RT-PCR may be pre-packaged in a kit and are further individualized, specialized, or geared towards a specific disease. For example, one such disease is COVID-19, caused by SARS-CoV-2. One kit that is specialized for SARS-CoV-2 and may be used to extract RNA and prepare for RT-PCR in carrying out the method of the invention is the TaqPath™ COVID-19 Combo Kit made and sold by ThermoFisher Scientific of 68 Third Avenue. Waltham, MA USA 02451. When using the TaqPath COVID-19 Combo Kit, nucleic acids are isolated and purified from the specimens using the MagMAX™ Viral/Pathogen Nucleic Acid Isolation Kit or the MagMAX™ Viral/Pathogen II Nucleic Acid Isolation Kit. Nucleic acid isolation can be performed manually or via an automated process using the KingFisher™ Flex Purification System (KingFisher). The RT-PCR reactions may then be prepared. During the PCR reaction, the nucleic acid is reverse transcribed into cDNA and amplified.

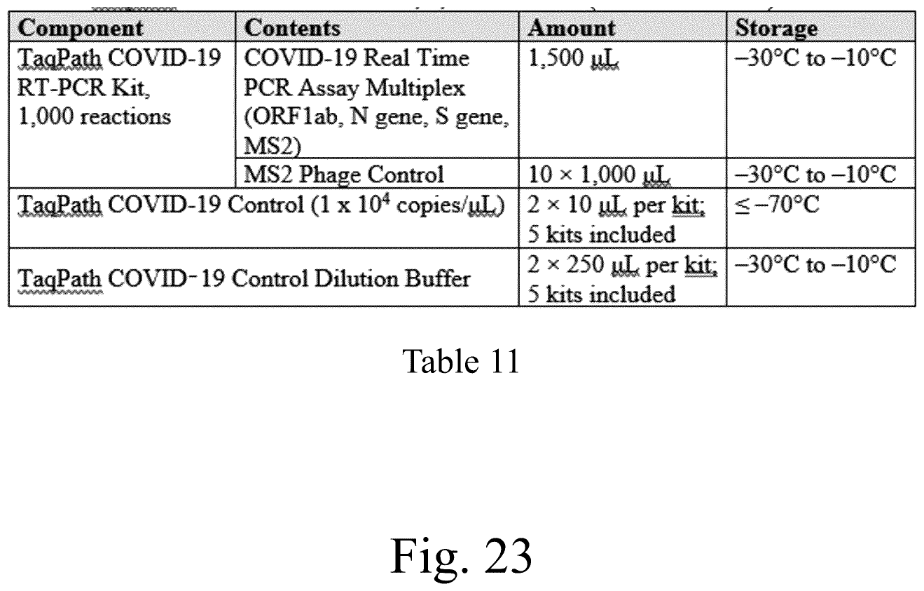

As shown in Table 11 of FIG. 23, the TaqPath COVID-19 Combo Kit includes reagents specialized for the genetic sequence of SARS-CoV-2 such as the ORF lab gene, nucleocapsid (N) gene, and spike (S) gene (i.e., the COVID-19 Real Time PCR Assay Multiplex), which is preferably stored at −30° C. to −10° C. The kit further includes MS2 Phase Control for an internal control to monitor reverse transcription and PCR inhibition. The kit may also further contain reagents such as a TaqPath COVID-19 control, preferably stored at temperatures equal to or less than −70° C., and TaqPath COVID-10 Control Dilution Buffer, preferably stored at −30° C. to −10° C.

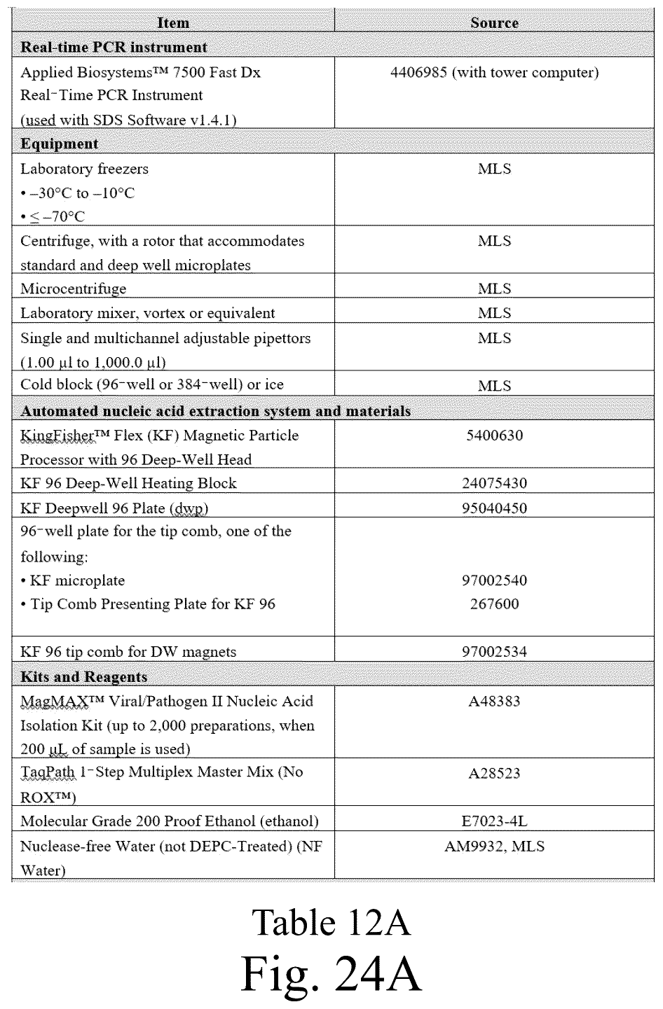

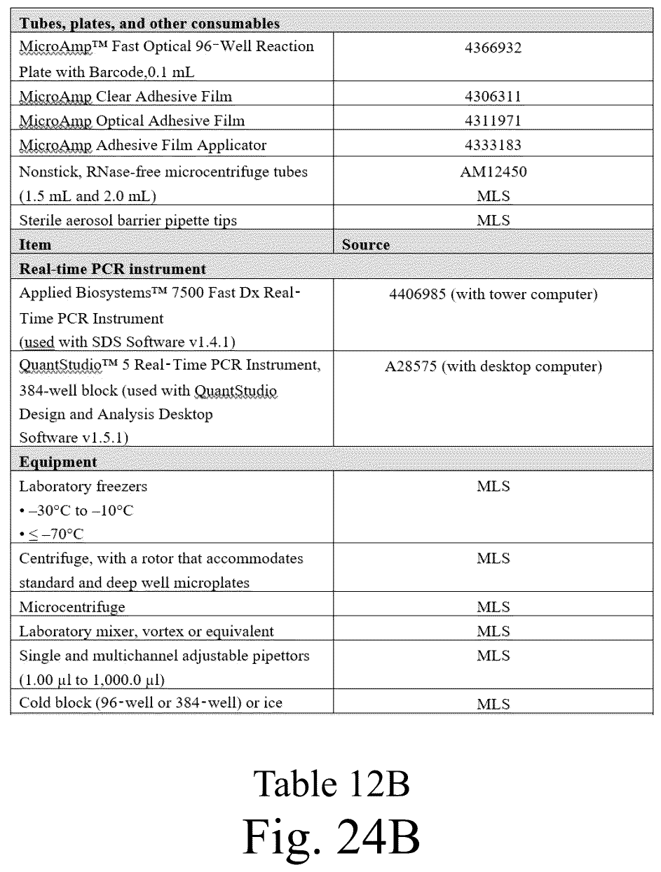

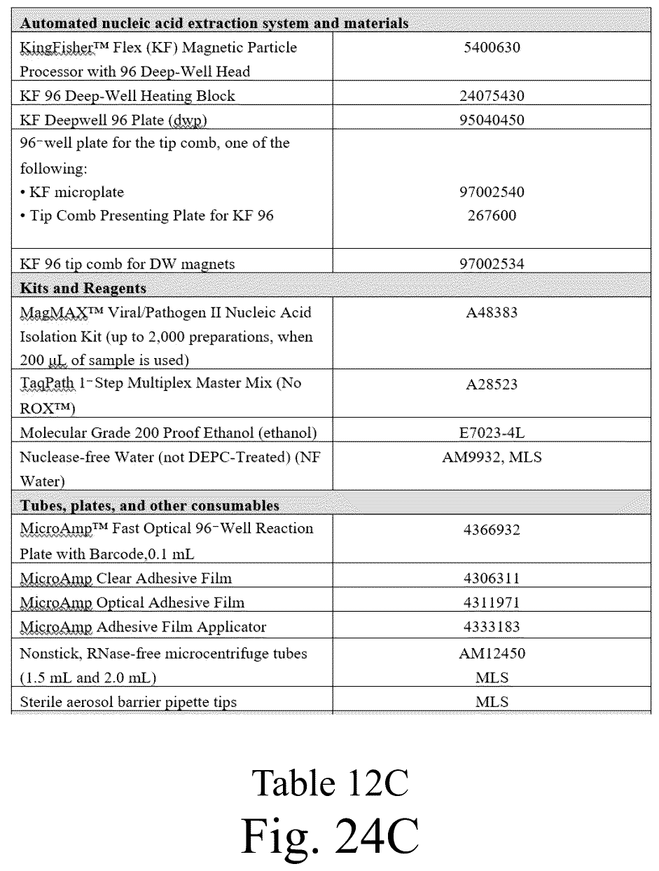

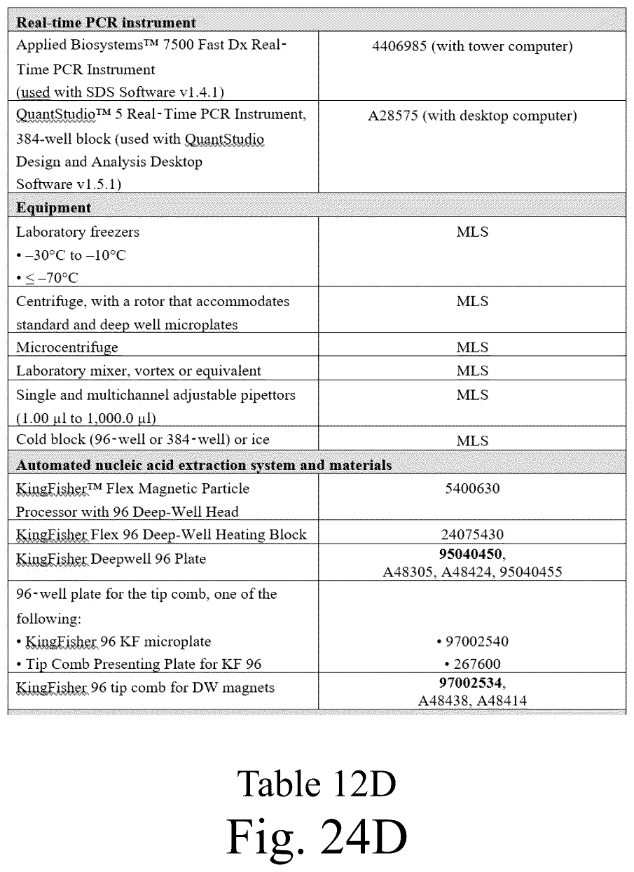

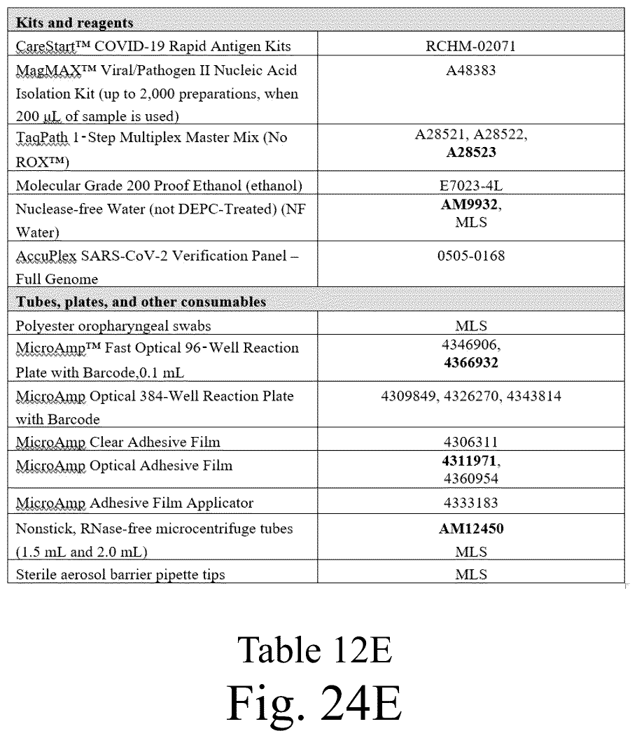

In addition to the material or regents contained in the TaqPath COVID-19 Combo Kit, the reagents, materials, or equipment, shown in Table 12A-12E of FIGS. 24A-24E, are further required for RNA extraction and purification and RT-PCR. The instruments needed include PCR machines (e.g., an Applied Biosystems 7500 Fast DX Real Time PCR Instrument with SPS software version 1.41 or a ThermoFisher QuantStudio™ 5 Real-Time PCR Instrument), 384-well block (used with QuantStudio Design and Analysis Desktop Software v1.5.1), listed in Table 12A-E of FIGS. 24A-24E. Necessary equipment includes laboratory freezers kept at temperatures between −30° C. to −10° C. and equal to or less than −70° C., microplate centrifuge(s) suitable for standard and deep-well microplates, microcentrifuges, laboratory mixer (vortex or the equivalent thereof), pipettors (from 1 μL to 1 mL), a cold block or ice/ice bucket, etc.

In order to extract and purify RNA from the pooled biological samples, an additional kit may be used. One such kit is MagMAX™ Viral/Pathogen II Nucleic Acid Isolation Kit, which may be included within the TaqPath COVID-19 Combo Kit. In addition to the kit for extracting RNA, the reagents TaqPath 1 Step Multiplex Master Mix (No ROX™), Molecular Grade 200 Proof Ethanol (ethanol), and Nuclease-free Water (not DEPC-Treated) (NF Water) are also required. The equipment needed for RNA extraction includes a KingFisher™ Flex (KF) Magnetic Particle Processor with 96 Deep-Well Head, KF 96 Deep-Well Heating Block, KF Deepwell 96 Plate (dwp), 96 well plate for the tip comb, either a KF microplate or Tip Comb Presenting Plate for KF 96, and KF 96 tip comb for DW magnets. Materials necessary for amplification and detection include a MicroAmp™ Fast Optical 96 Well Reaction Plate with Barcode 0.1 mL, MicroAmp Clear Adhesive Film, MicroAmp Optical Adhesive Film, MicroAmp Adhesive Film Applicator, Nonstick RNase-free microcentrifuge tubes (1.5 mL and 2.0 mL), sterile aerosol barrier pipette tips.

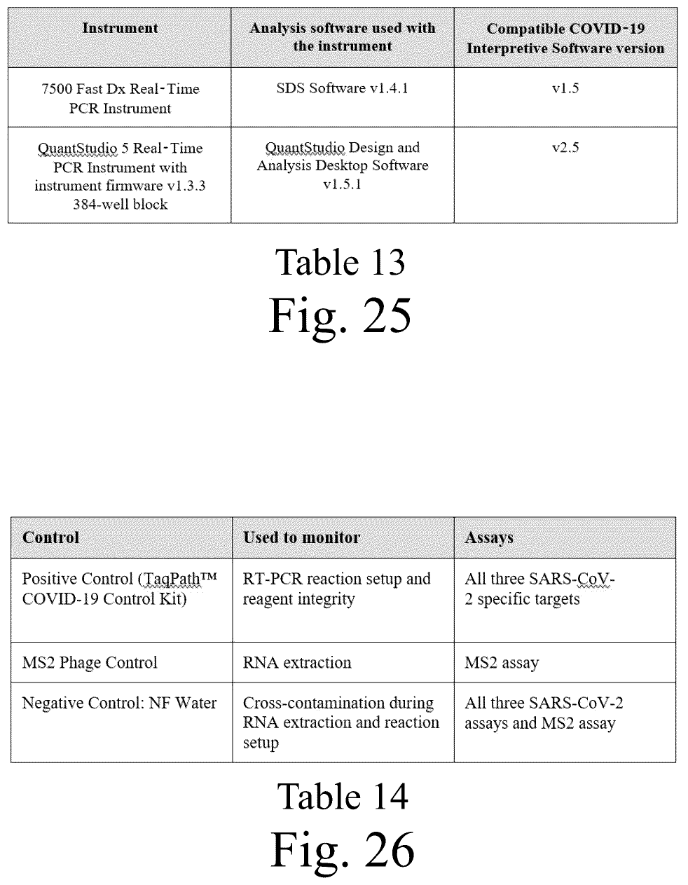

Table 13 of FIG. 25 shows the instrumentation and compatible software versions for each one of two preferred diagnostic pathogen testing instruments, namely an Applied Biosystems 7500 Fast Dx Real Time PCR instrument and Applied Biosystems QuantStudio 5 Real-Time PCR Instrument both of which made and/or sold by Thermo Fisher Scientific of Waltham, Massachusetts, well suited for use in performing PCR diagnostic pathogen testing on the processed and refined pooled samples, preferably concentrated processed and refined pooled samples, in carrying out a pooled sample diagnostic pathogen testing method in accordance with the present invention.

In order to extract RNA from the pooled samples (200-400 either an automated or a manual method may be used. In embodiments where the RNA extraction is automated, the components from the MagMAX™ Viral/Pathogen Nucleic Acid Isolation Kit or the MagMAX™ Viral/Pathogen II Nucleic Acid Isolation Kit may be used. The instrument for extracting RNA may be prepared by ensuring the KingFisher™ Flex Magnetic Particle Processor with 96 Deep-Well Head is set up with the KingFisher™ Flex 96 Deep-Well Heating Block, and the program MVP_2Wash_200_Flex program has been downloaded from ThermoFisher and loaded onto the instrument. The processing plates may then be prepared, which include three deep-well 96 plates and one 96 KF microplate. The three deep-well 96 plates may be labeled wash 1 plate, wash 2 plate, and elution plate and placed so that wash 1 plate is located in the plate 2 position, wash 2 plate is located in the plate 3 position, and the elution plate is located in the plate 4 position. The tip comb plate may be located in the plate 5 position and placed for DW magnets in a KingFisher 96 KF microplate. Wash 1 plate may further include 500 μL of wash solution in each well, while wash 2 plate and elution plate includes 1000 μL of 80% ethanol and 50 μL of elution solution in each well, respectively. The plates may then be covered with a temporary seal (e.g., MicroAmp™ Clear Adhesive Film), then stored at room temperature for up to 24 hours.

In order to prepare the binding beads, all the nucleic acid magnetic beads may be vortexed to ensure that the bead mixture is homogeneous. 530 μL of binding solution and 20 of nucleic acid magnetic beads may be prepared to form 550 μL of binding bead mix. The binding bead mix should be mixed well by inversion before being stored at room temperature, while the sample plate is prepared. The sample plate (with 200-400 μL of pooled sample) may be prepared by adding 5-10 μL of proteinase K to each well of the sample plate, where the sample plate is preferably a deep-well 96 plate. 200-400 μL of the pooled sample is added to each well, while 200-400 μL of nuclease-free water (not DEPC-treated) is added to an empty well to serve as a negative control. Before 275-550 μL of the binding bead mixture is added to each sample well, including the wells with the pooled samples as well as the negative control, the binding bead mixture should be inverted to mix the contents of the binding bead mixture. Finally, add 5-10 μL of MS2 Phage Control to each sample well as well as the negative control to serve as an internal control.

The program MVP_2Wash_200_Flex on the KingFisher Flex Magnetic Particle Processor with 96 Deep-Well Head should be selected. After the run has been started, the prepared plates should be loaded onto the instrument in the position indicated by the instrument. After the run is completed, taking approximately 24 minutes, the elution plate should be immediately removed and covered with MicroAmp clear adhesive film to prevent evaporation. The elution plate should be placed immediately on ice (or in 4° C.) for use in real-time PCR.

To prepare the RT-PCR reactions (for an original 200-400 μL pooled sample) and using the TaqPath COVID-19 Combo Kit, the reagents should be gently vortexed to mix the reagents and then centrifuged briefly to collect the liquid at the bottom of the tube. TaqPath COVID-19 Control should be diluted by adding 98 μL of TaqPath COVID-19 control dilution buffer and 2 of TaqPath COVID-19 Control into a first microcentrifuge tube and mixed. 87.5 μL of COVID-19 control dilution buffer and 12.5 μL of the dilution located in the first microcentrifuge tube should be added to a second microcentrifuge tube and mixed to create the working positive control.

In order to prepare the reaction mix for each sample well and controls in a reaction plate for RT-PCR on the ABI 7500 Fast Dx, 6.25 μL of TaqPath 1-step multiplex master mix, 1.25 COVID-19 real time PCR assay multiplex, and 7.5 μL of nuclease-free water should be added to create a reaction mixture of 15 μL. The 15 μL of reaction mixture should be pipetted into each well, containing either a sample or control, of a MicroAmp Fast Optical 96-well reaction plate with barcode 0.2 mL. In order to prepare the reaction mix for each sample well and controls in a reaction plate for RT-PCR on the QuantStudio 5, 5.0 μL of TaqPath 1-step multiplex master mix, 1.0 μL COVID-19 real time PCR assay multiplex, and 4 μL of nuclease-free water should be added to create a reaction mixture of 10 μL. The 10 μL of reaction mixture should be pipetted into each well, containing either a sample or control, of a MicroAmp Optical 384-well reaction plate with barcode 0.1 mL.

The sealed plate containing the purified RNA from the biological samples and negative control from the RNA extraction procedure should be gently vortexed to mix the liquid and then centrifuged briefly to collect liquid at the bottom of the plate before being unsealed. 10 μL of the purified RNA sample should be added to each well and 10 μL of the negative control should be added to the negative control well, while 2 μL of the positive control and 8 μL of nuclease-free water should be added to the positive control well. Thus, as shown in Table 14 of FIG. 26, the RT-PCR reaction will include three controls—the positive control used to monitor the RT-PCR reaction setup and reagent integrity, the MS2 Phage control to monitor RNA extraction, and the negative control used to monitor for cross-contamination during RNA extraction and PCR reaction set up. Once all the RNA and controls have been added to the reaction plate, the plate is sealed with a MicroAmp Optical Adhesive Film. The sealed reaction plate can then be vortexed to ensure that the contents of each well are mixed, before being centrifuged to remove bubbles and collect the liquid at the bottom of the well.

In order for amplification and detection to be performed on the instrument, the PCR machine must be set up to determine a run time or a run program. In one embodiment, the PCR machine may be set to run a UNG incubation step at 25° C. for two minutes, a reverse transcription step at 53 C for ten minutes, an activation step at 95 C for two minutes before repeating a denaturation step at 95 C for three seconds and an anneal/extension step at 60 C for thirty seconds forty times.

### VI. Results

After running the PCR machine, the data may be analyzed in an automated fashion through a computer program, Applied BioSystem's COVID-19 Interpretive Software.

In one embodiment, the database of samples is connected to a virtual machine through a Linked Server setup within the database. The computer program may be scheduled to run on an hourly basis. When running, the computer program preferably saves the date and time. In one embodiment, the date and time may be saved when the program begins running. The program identifies the pool identification, results, and batch identification for all the pool samples that resulted within the time period between the current time and the last time that the program was run.

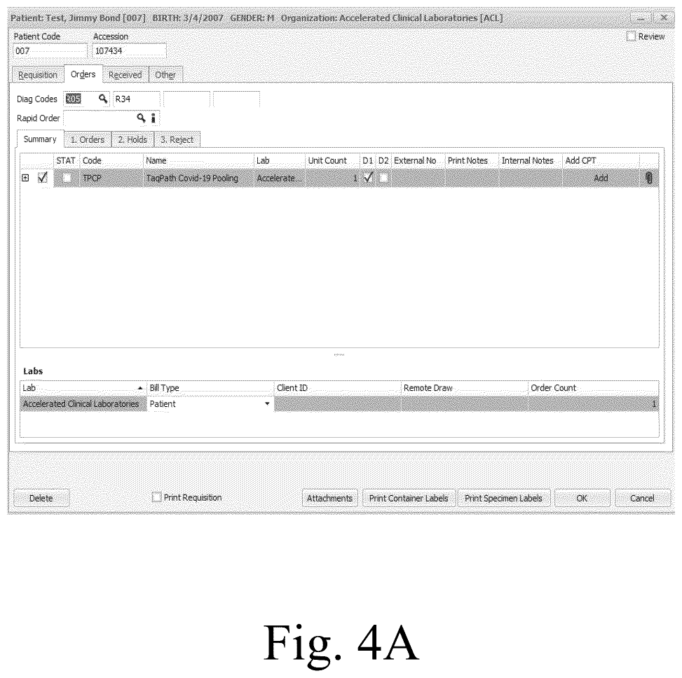

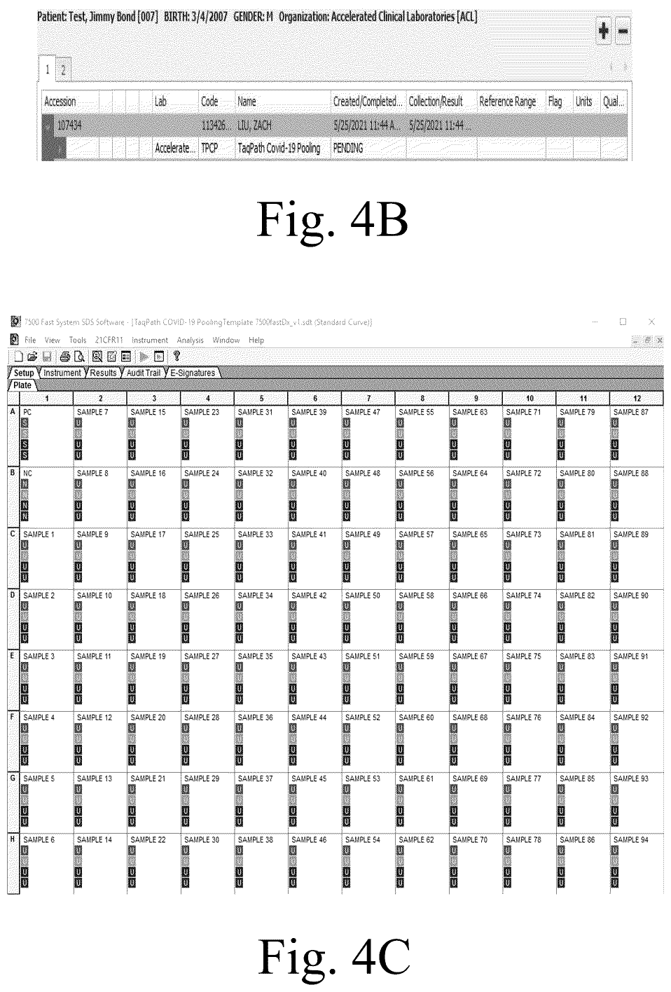

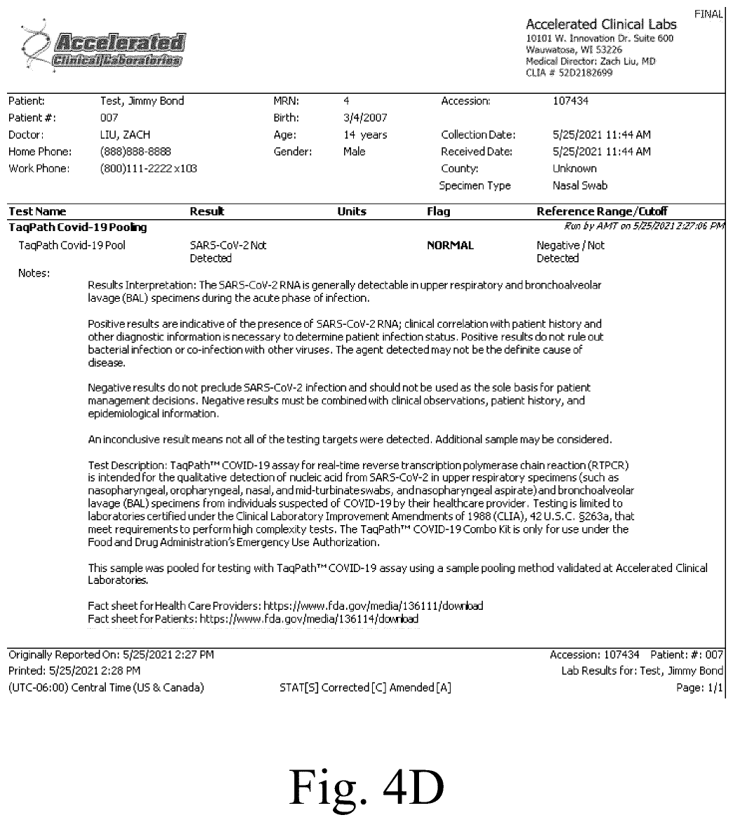

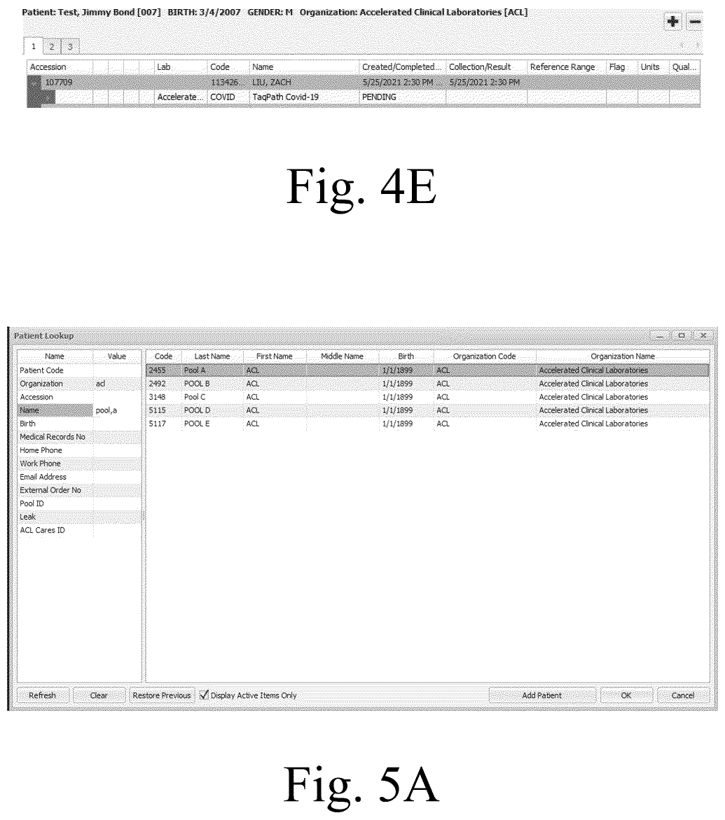

As seen in FIGS. 4A-4E, in one embodiment, when an individual is to be tested using wet media sample pooling, a requisition (i.e., a TaqPath Covid-19 Pooling Order) is added for the individual or patient. The pool order is then listed as “PENDING” until pool PCR results are imported.

Samples are scanned into a LABDAQ batch and the Batch Worklist .CSV containing accession numbers for all samples is exported. Pooled samples are entered into the PCR instrument run software as “SAMPLE 1, SAMPLE 2” . . . up to “SAMPLE 94” per batch. The instrument run file is imported into Thermo COVID-19 Interpretive Software and analysis .CSV exported. LABDAQ Batch Worklist is copied into “LABDAQ Worklist” tab and analysis .CSV export into “7500 Analysis” tab of Deconvolution macro to apply data from pool to each individual accession. Deconvoluted data is generated in “Deconvoluted Data” tab and exported into a new file for import into LABDAQ.

Upon import to LABDAQ, pool results trigger the following: (a) negative pools are reported as “Not Detected” directly from the TaqPath Covid-19 Pooling order and reports are generated with the note that results are from pooled testing; and (b) positive and inconclusive pool results are applied to the constituents of the pool, and each individual accession automatically reflexes to the standard TaqPath Covid-19 (COVID) panel.

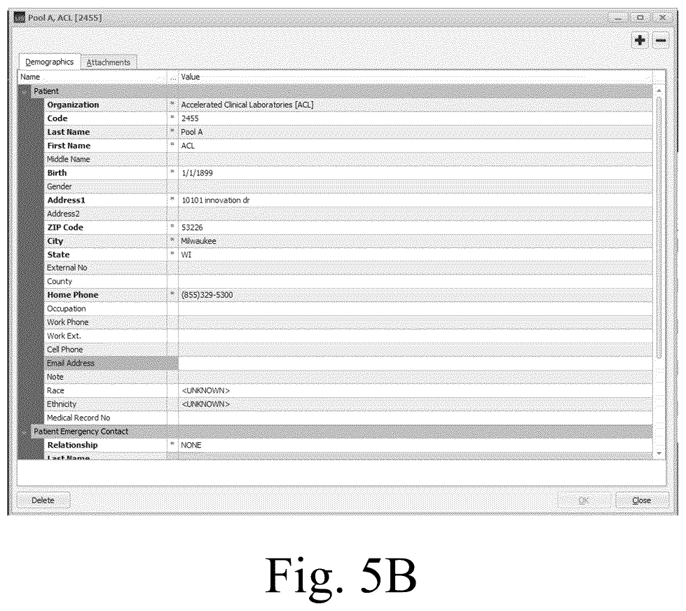

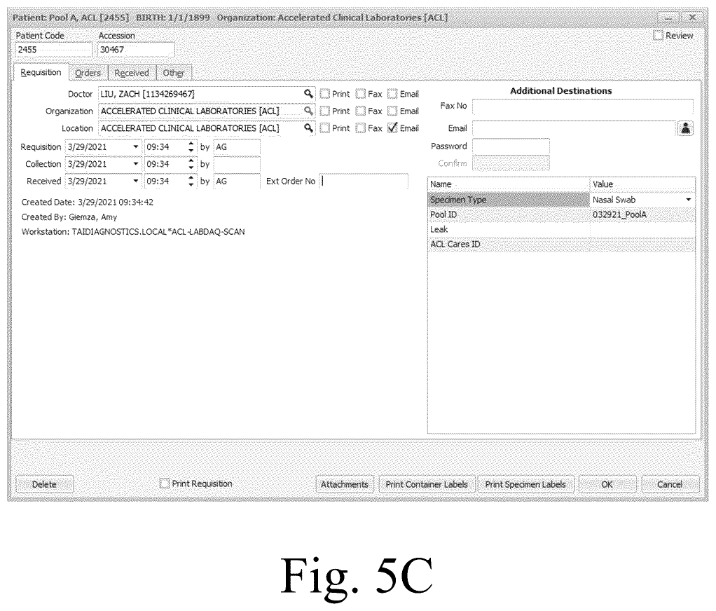

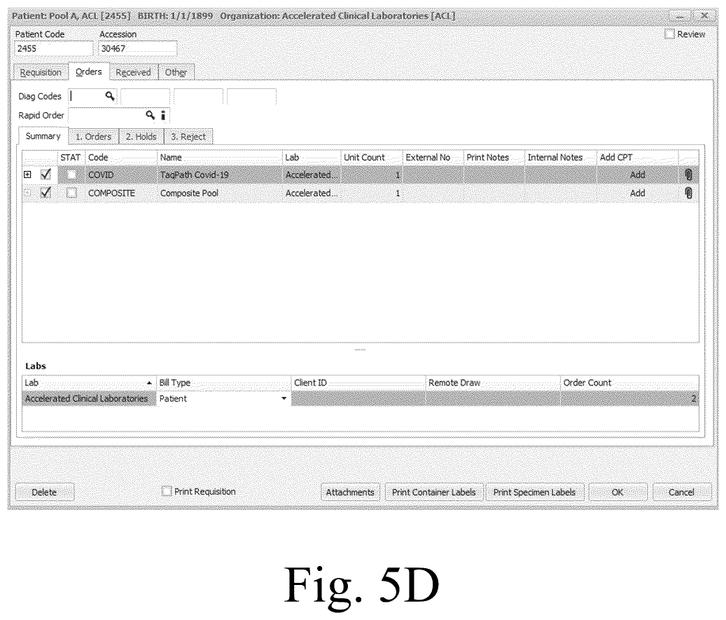

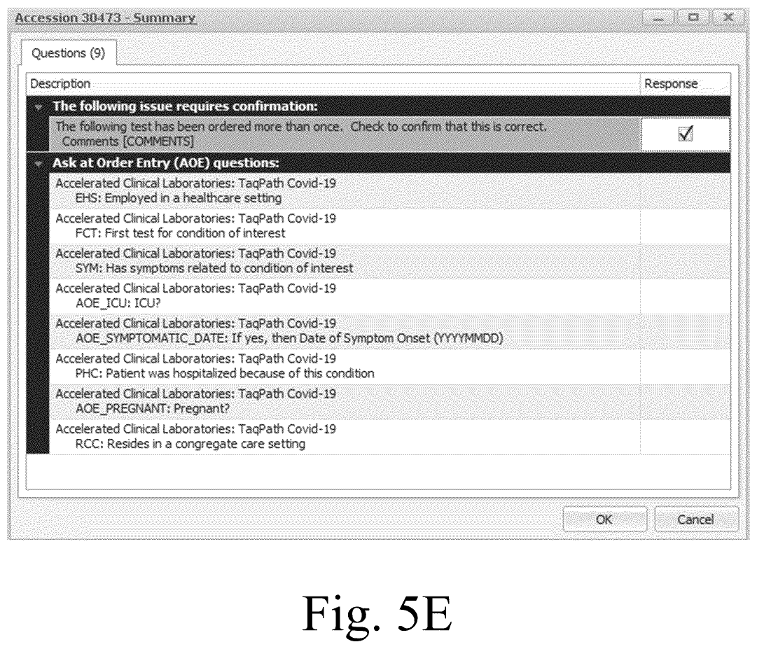

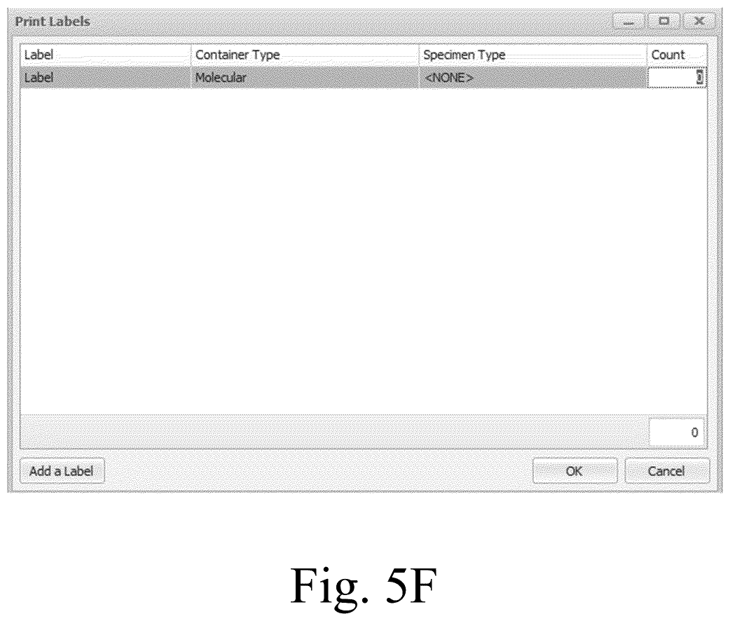

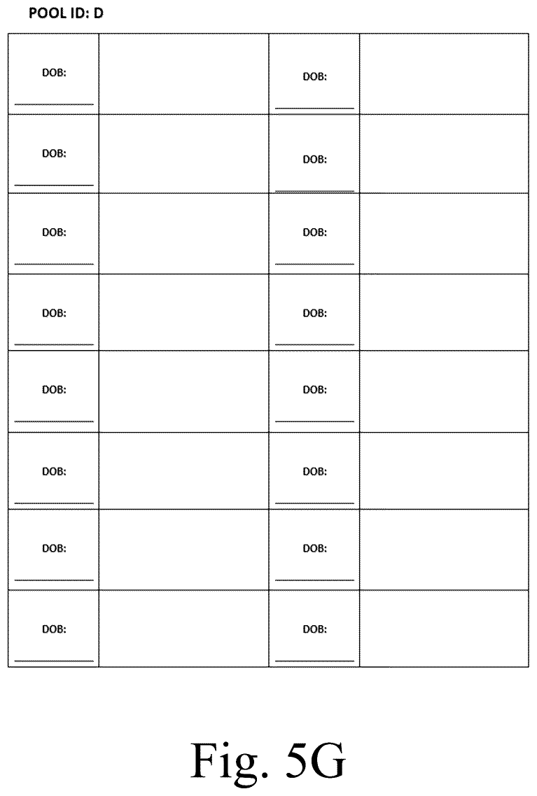

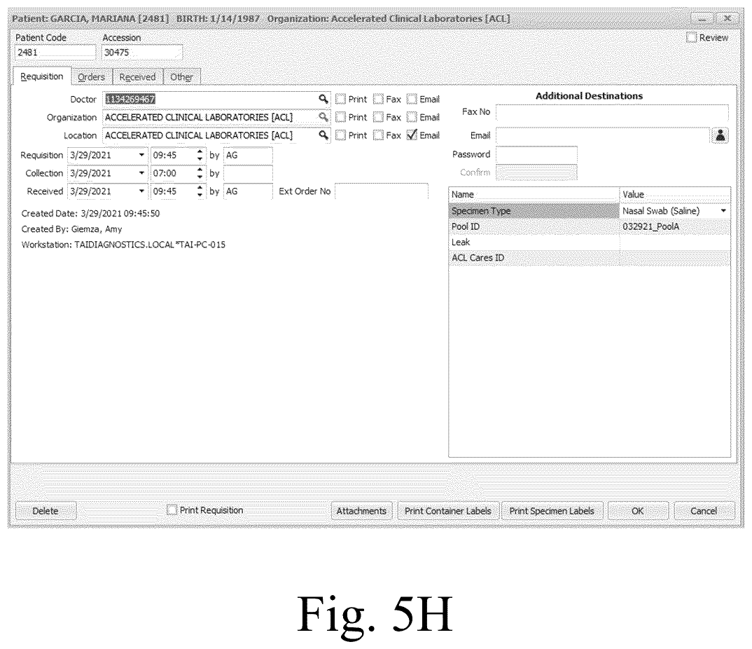

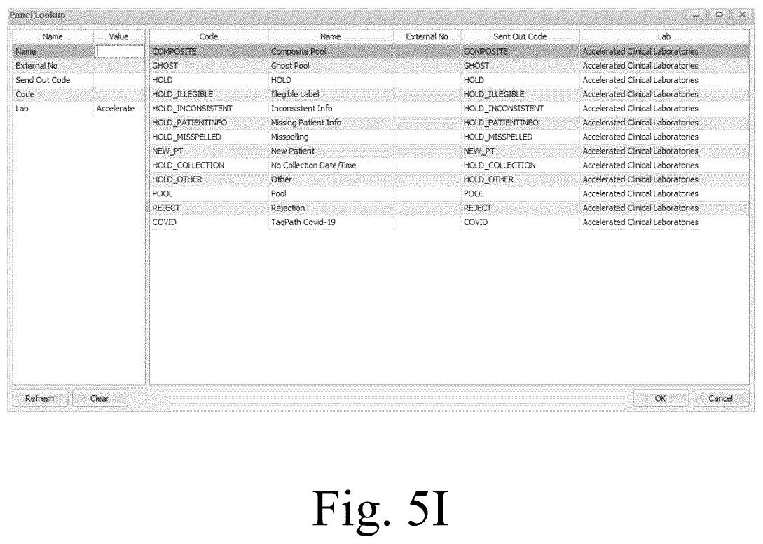

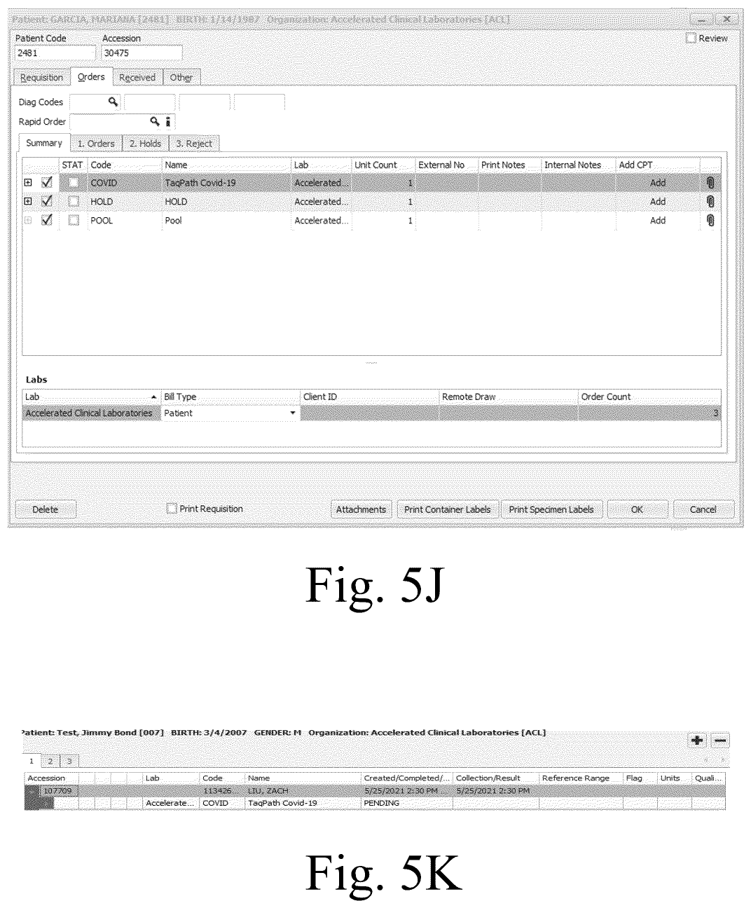

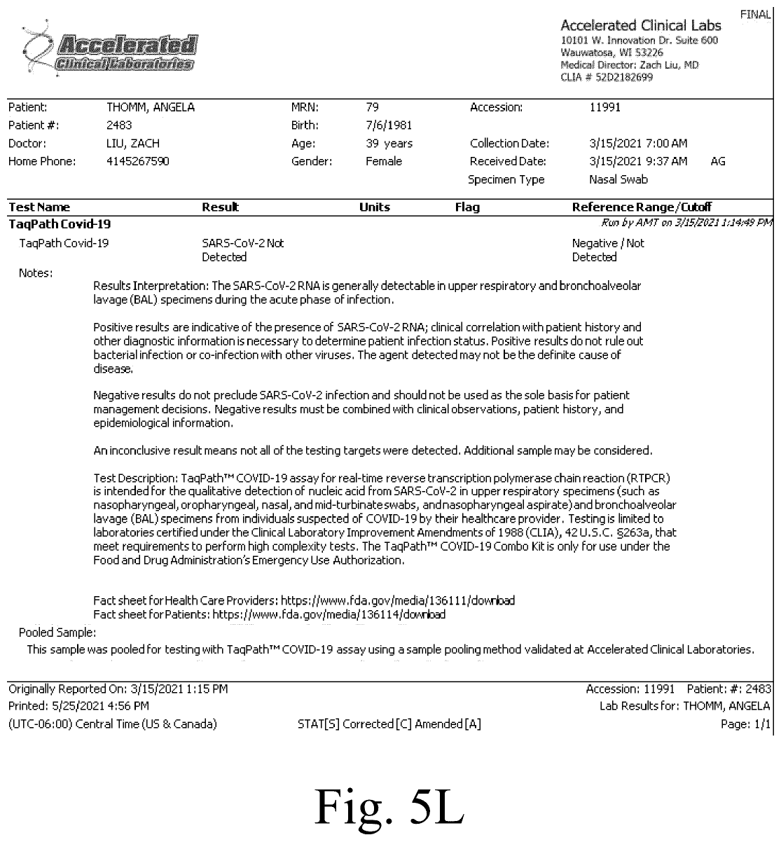

As seen in FIGS. 5A-5L, in one embodiment, when an individual is to be tested through dry sample pooling, a requisition (i.e., a TaqPath Covid-19 Pooling Order) is added for the individual or patient. The individual or patient should be searched for in “Patient Lookup,” for previously created pools by facility, in LABDAQ. If a previously created pool exists, open associated pool patient. If no previous pool patient exists, proceed to “Add Patient”. If “Add Patient” is selected, add the new pool under the appropriate organization. The last name field should be “Pool A-Z” and the first name field should be the short name of the organization. DOB should be Jan. 1, 1899 and the address and phone number should be that of the facility. Other labels may be substituted or altered in alternative embodiments.

Requisition information should be entered in accordance with normal accessioning protocol, including date of collection. Specimen type will be “Nasal Swab” and the Pool ID should be selected from the drop-down list according to date and Pool letter assigned. The pool ID will be created in LabDAQ>System Setup>User Defined Field Setup under the Pick Items list. Pool IDs will be generated with the following format MDYY Pool A-Z.

Under “Orders” tab within the “Orders” menu, check the box for [COVID]. Select the magnifying glass to open panel lookup. Select the COMPOSITE code and click OK to apply to order. Close requisition by selecting “Print Specimen Labels.” When the accession summary window appears, select the check box to confirm that more than one test has been assigned and select OK. Type 3 into the count field when the print labels window appears and select OK to print the labels in triplicate. Two labels are sent to the lab and the third label is attached to the top of the Pool Requisition form. The third label is placed at the top of the page next to “POOL ID”.

In “Patient Lookup” search for remaining patients from pool. Open associated patients and proceed to add requisitions or if no previous pool patient exists, proceed to add patient then add requisition. Enter requisition information for each individual in accordance with normal protocol. Under “Orders” tab within the “Orders” menu select the magnifying glass to open panel lookup. Hold control and select the POOL, HOLD and COVID codes and click OK to apply to order. The summary menu should appear as pictured prior to printing. Close requisition by selecting “Print Specimen Labels.” If the sample came in with a physical requisition form, mark for review and print two labels applying the first to the top right corner of the physical requisition form and the second to the composite requisition form. If the patient was previously logged and they were not received with a physical requisition form, print one label for the composite form. Scan the Composite Form and Add to Composite Requisition.

The order is listed as “PENDING” until pool PCR results are imported. Pool samples are scanned into a LABDAQ batch and the Batch Worklist .CSV containing pool accession numbers is exported. Pool accession numbers are entered into the PCR instrument run software. The instrument run file is imported into ThermoFisher COVID-19 Interpretive Software and analysis .CSV exported by the same process as individual samples. Analysis .CSV export is imported into LABDAQ. Scheduled database task runs once per hour. Task checks LABDAQ for new results marked as pooled samples which are “Not Detected”.

If new results are found then the program searches LABDAQ for constituent samples in same pool and, for each POOL ID, creates a result .CSV with the same structure as the Analysis .CSV and uses “Not Detected” as the result value for each constituent sample. Each new result .CSV is imported into LABDAQ and reports are generated for negative samples with a pool disclaimer. Positive and inconclusive pools must be deconvoluted using a second, wet media sample collected at the same time as the dry swab sample.

Alternatively, the computer software saves the values for the pool identification, results, and batch identification in at least one first temporary table. Indexes are then generated for each of the each of the at least one first temporary table. The values are then combined into a signal table, based on accession number, and then stored in a second temporary table. The individual samples, its accession number, and pool identification are then placed in a third temporary table. An index is generated for the third temporary table. The program then creates a .CSV file from a template using the interpretive software.

The program goes through each pool identification and adds a row for each individual sample, including the batch identification and pool sample result. Negative results are reported as “SARS-CoV-2 Not Detected.” Negative results may further be reported with a disclaimer such as “This sample was pooled for testing with TaqPath COVID-19 assay using a sample pooling method validated at Accelerated Clinical Laboratories.” A new .CSV file for each pooled sample with a negative/undetected result is then created. The .CSV file is then copied to an Analyzer Result folder on the ACL-LABDAQ-SCAN (same machine that's running the LABDAQ database). Samples with positive or inconclusive results are deconvoluted and reported via ACL's standard TaqPath COVID-19 workflow, as will be discussed in more detail hereinafter. False-negative results may arise from: (1) Improper sample collection; (2) Degradation of the SARS-CoV-2 RNA during shipping/storage; (3) The presence of RT-PCR inhibitors; and (4) Mutation in the SARS-CoV-2 virus.

### VII. Validation

In order to test the viability of the results, each Pool Tube (of the pooled samples) was tested using the CareStart COVID-19 Rapid Antigen Test Kit following manufacturer's instructions for use and the TaqPath COVID-19 Combo Kit per standard operating protocol. The CareStart test contains a built-in, internal procedural control. A red-colored line appearing in the control region “C” is designed as an internal procedural control. The appearance of the procedural control line indicates that sufficient flow has occurred, and the functional integrity of the test device has been maintained. The presence of acceptable control lines was documented for all CareStart assays run as part of this validation. External controls are used to demonstrate that the test device and test procedure perform properly. The manufacturer recommends that positive and negative external control swabs are run once with each new lot, shipment and user. External positive and negative control swab data was documented as acceptable for all runs.

In the TaqPath COVID-19 Combo Kit, a minimum of one negative control (NC) and one positive control (PC) must be included with each run. Additional NC wells must be run for each extraction that is represented on a 384-well RT-PCR plate. All control wells passed QC criteria for validation runs. Validation of results is performed automatically by the Applied Biosystems™ COVID-19 Interpretive Software based on performance of the PC and NCs.

In addition to testing whether the results themselves are valid, additional validation tests were used to determine whether sufficient viral particles were recovered. In order to assess the recovery of viral particles from polyester oropharyngeal swabs, triplicate swabs were charged with 200 μl of AccuPlex SARS-CoV-2 Verification Panel standards with stock concentrations of 100,000 and 10,000 copies/ml, the equivalent of 667 copies and 66.7 copies respectively per extraction well at 100% recovery. Each charged swab was placed into a 50 ml conical tube containing 6 ml of saline. An additional 24 clean, dry swabs were added to each conical to simulate a 25 swab Pool Tube. 200 μl from each pool was assayed per standard procedure and Ct values determined.

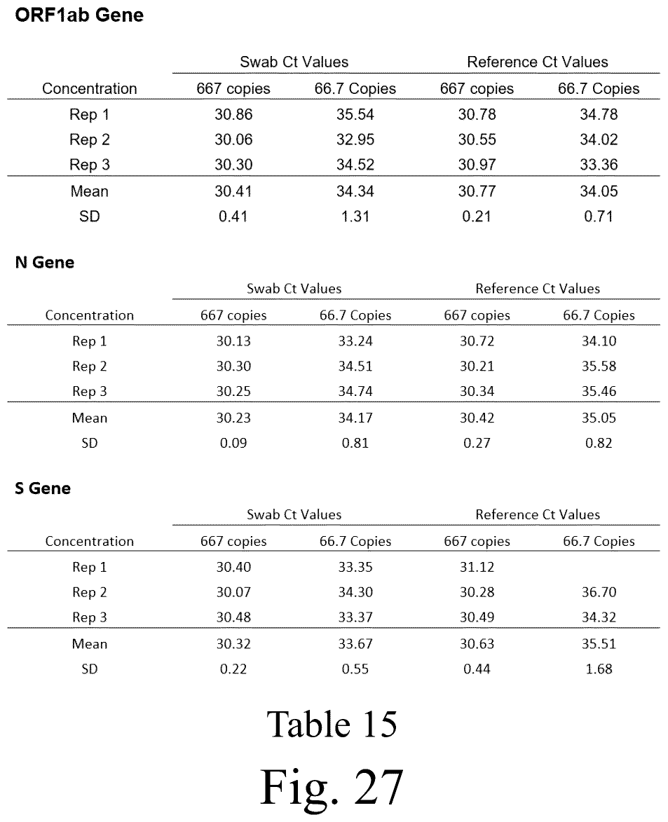

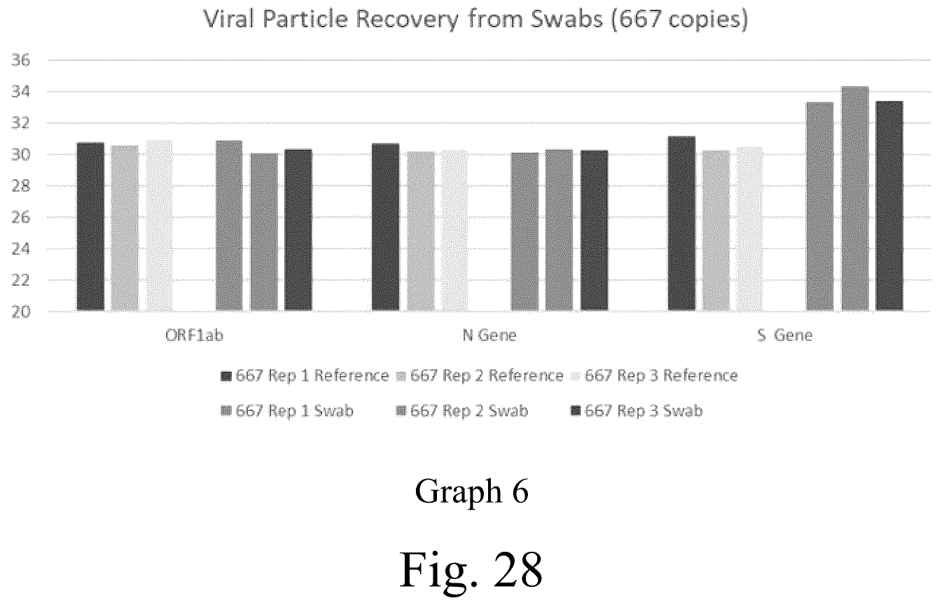

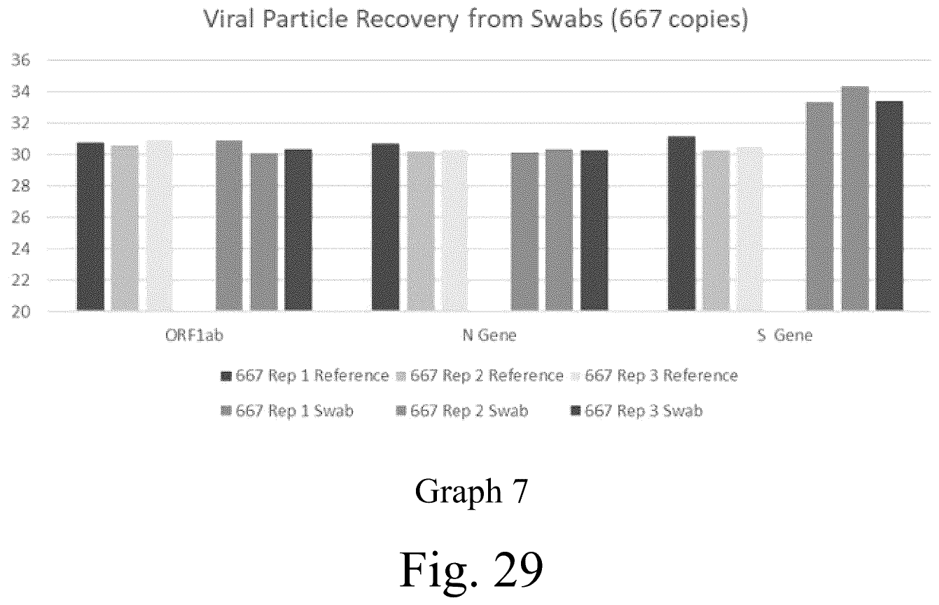

For comparison, 66.7 μl of 10,000 copies/ml standard was added to 133.3 μl of saline in triplicate extraction wells (667 copies) and 66.7 μl of 1,000 copies/ml standard was added to 133.3 μl of saline in triplicate extraction wells (66.7 copies). Extraction and RT-PCR were performed per standard procedures. Mean Ct's from the diluted SeraCare standards were compared to mean Ct's from Pool Tubes in the immediately preceding paragraph above. Mean Ct values for contrived swab pools were within 2 Ct's of comparison samples, meeting acceptance criteria. As can be seen in Table 15 of FIG. 27, Graph 6 of FIG. 28, and Graph 7 of FIG. 29, more than sufficient viral particles were recovered.

Graph 6 of FIG. 28 provides a comparison of values for Mean Cycle Thresholds, Cts, for swabs charged with 20,000 copies of Accuplex SARS-CoV-2 standard versus or compared to a diluted standard for detection of the three different genetic templates of ORF lab, N Gene, and S Gene representative of the pathogenic genetic material of the SARS-CoV-2 pathogen being screen by pooled sample pathogen diagnostic testing.

Graph 7 of FIG. 29 provides a comparison of values for Mean Cycle Thresholds, Cts, for swabs charged with 2,000 copies of Accuplex SARS-CoV-2 standard versus or compared to a diluted standard for detection of the three different genetic templates of ORF lab, N Gene, and S Gene representative of the pathogenic genetic material of the SARS-CoV-2 pathogen being screen by pooled sample pathogen diagnostic testing.

As such, further validation studies were completed to identify whether pooled samples, containing at least one positive biological sample, would be identified. In order to test whether pooled samples provided accurate results, twenty positive clinical samples with Ct values spanning the analytical range for the TaqPath assay were combined with four negative samples and tested as a pool in a single well of an extraction plate. The pooling method would be considered acceptable for diagnostic use if 19 of the 20 pooled samples test positive.

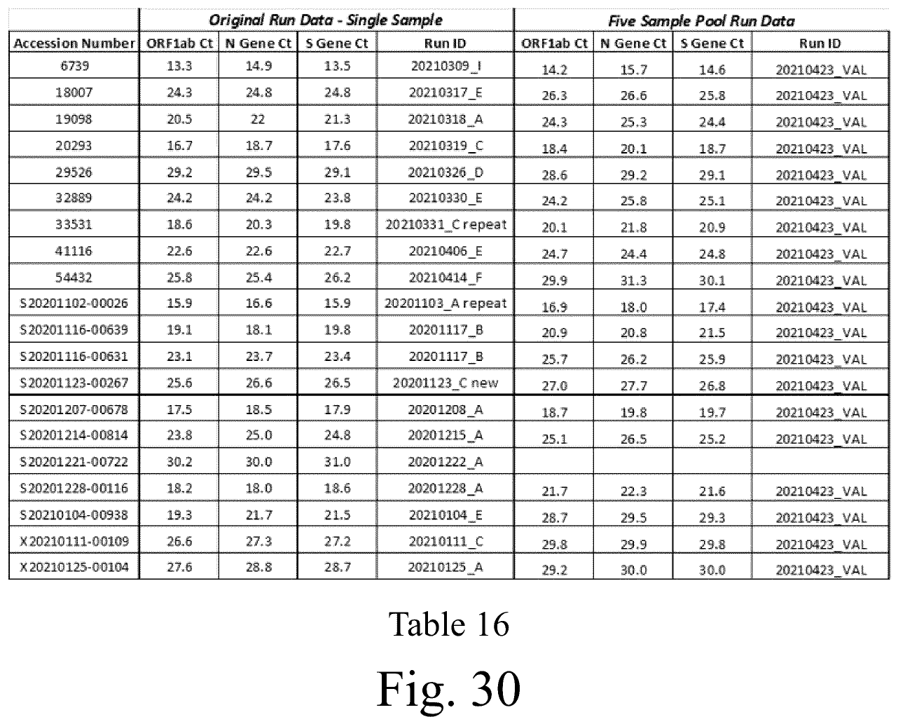

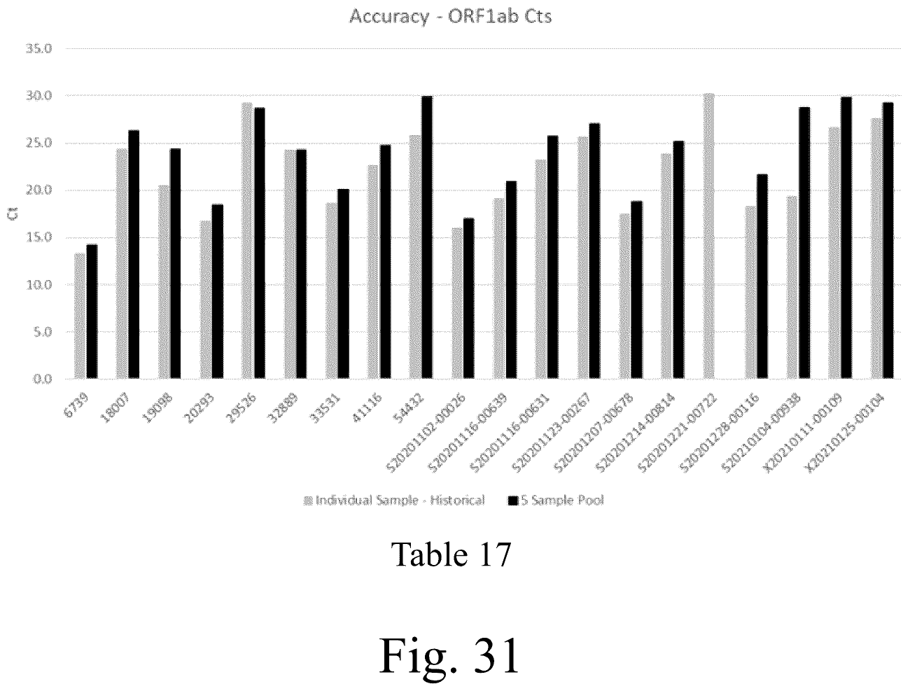

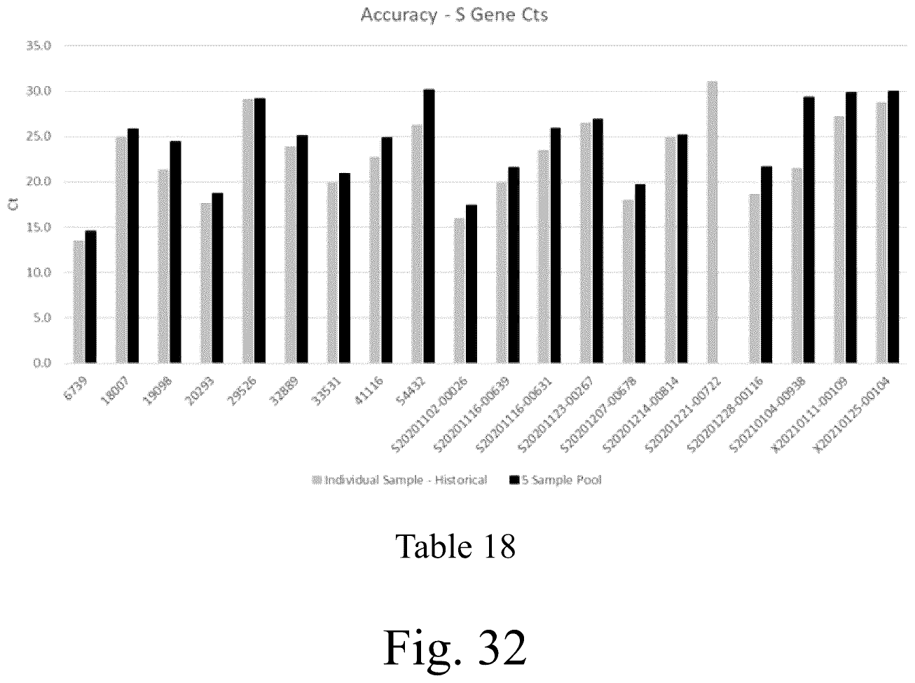

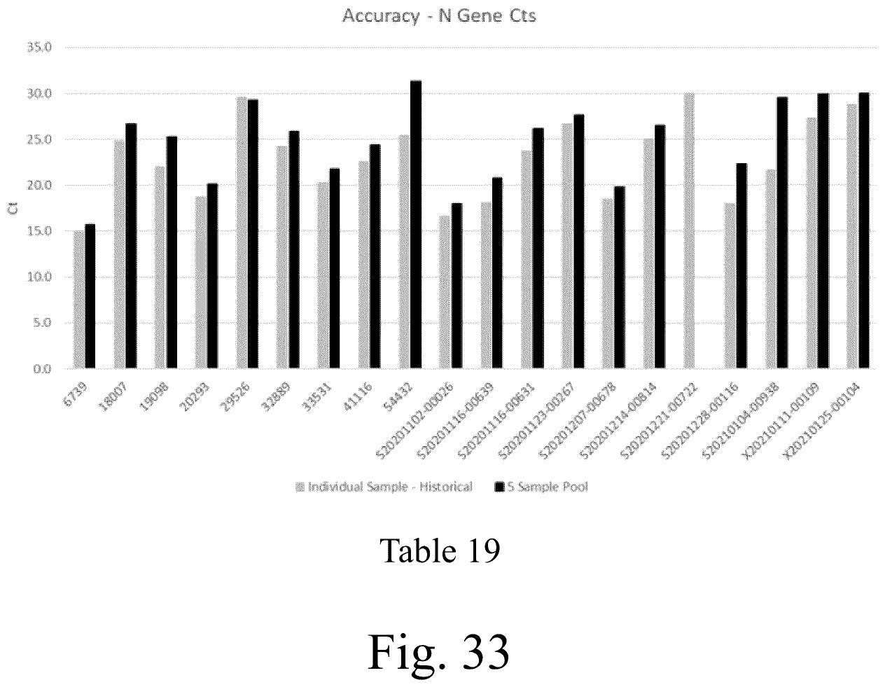

Ct data for positive pool samples is recorded in Table 16 of FIG. 30. Nineteen (19) of twenty positive pool samples advantageously tested positive, thereby meeting the acceptance criteria.

In order to further test accuracy, 20 pools made by combining four known negative clinical samples with one known positive sample per pool were also tested. All 20 negative sample pools tested negative also meeting the acceptance criteria outlined (i.e., 95% accurate).

In addition to testing for whether sufficient viral particles may be obtained from biological samples and the accuracy of such tests, the precision of the tests were also analyzed.

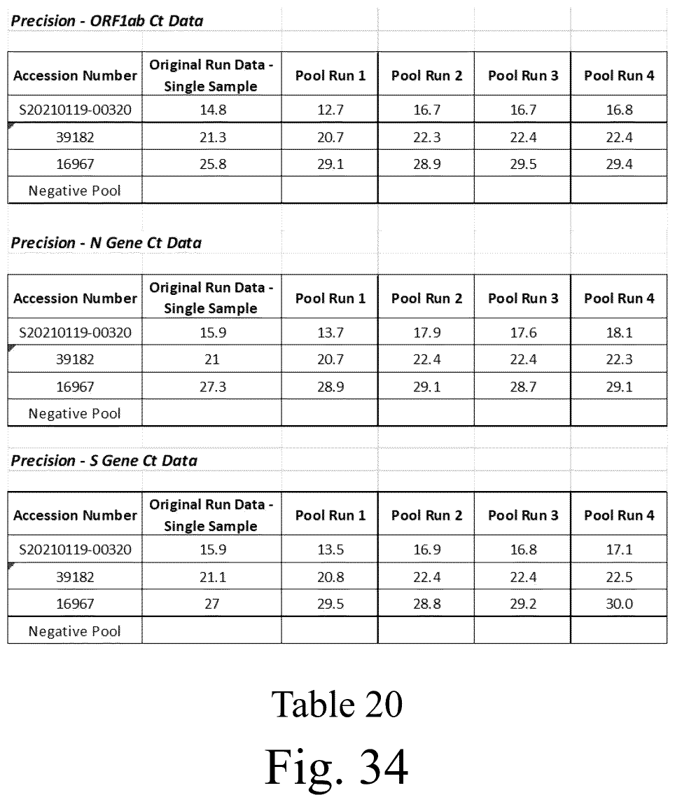

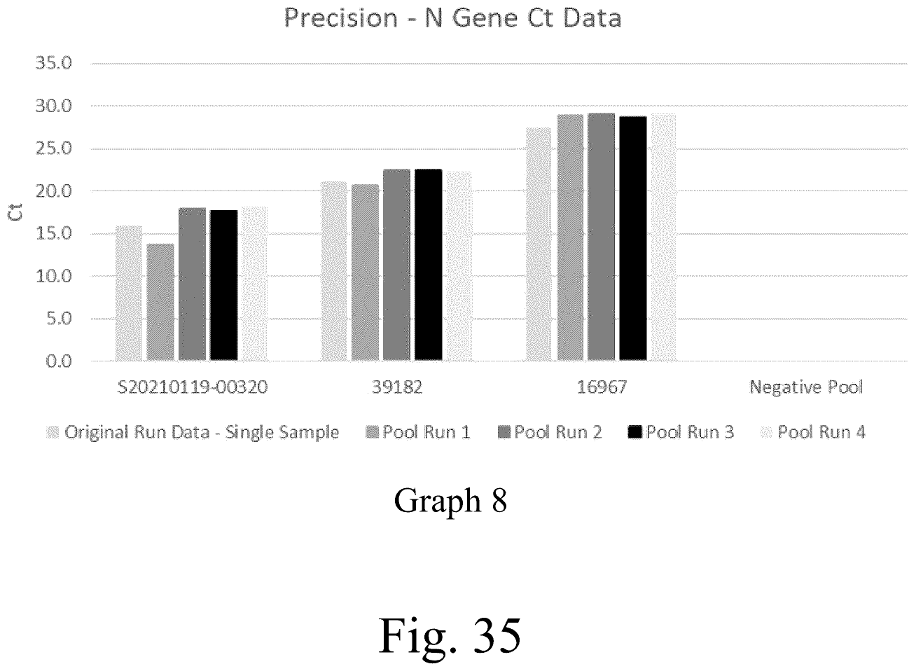

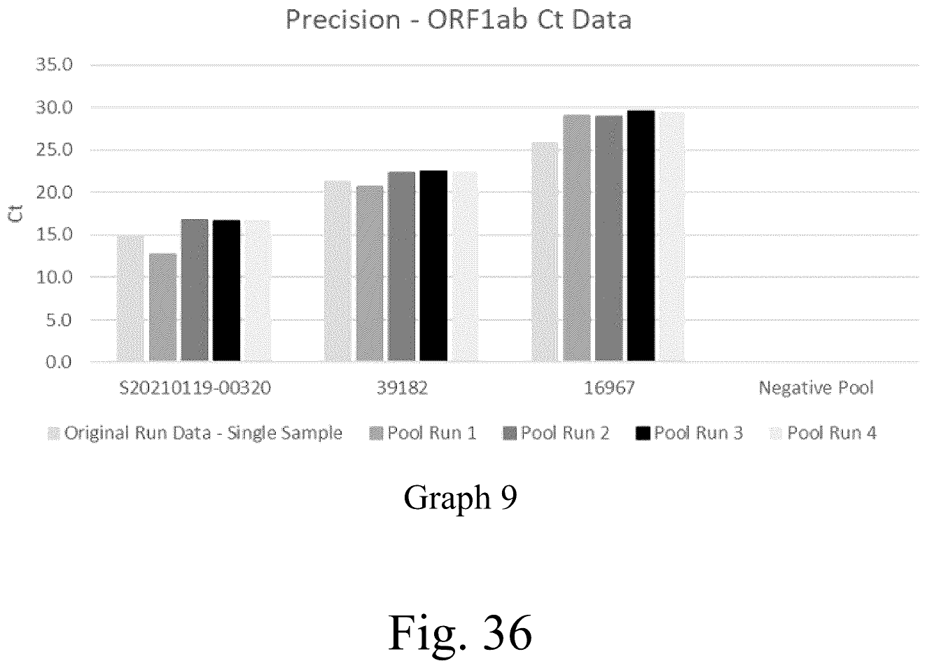

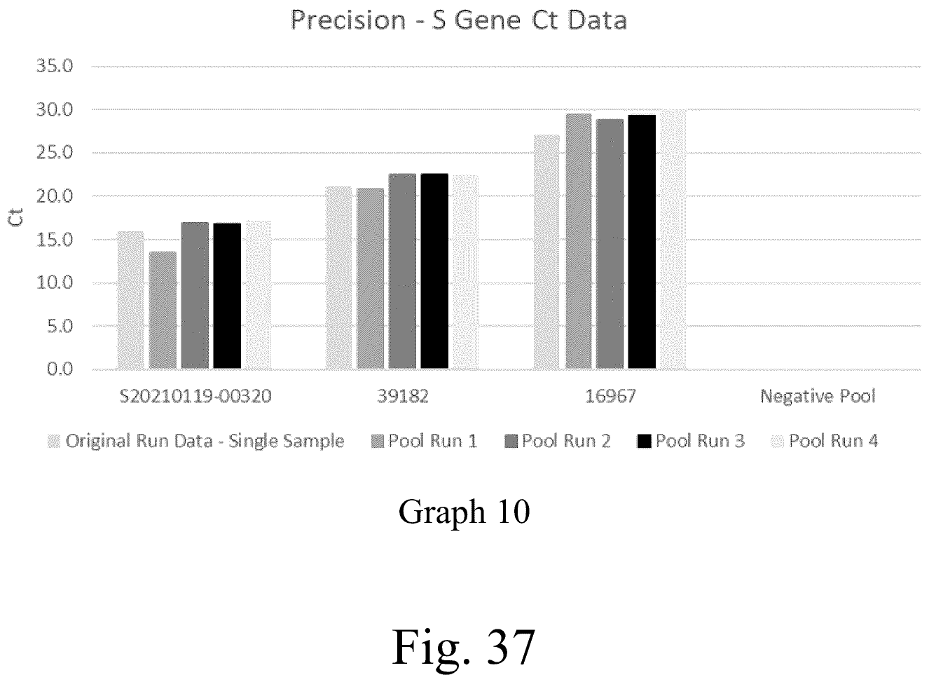

Three positive and one negative clinical sample were each combined with four negative samples and tested as a pool across three days, one run per day as described previously. Ct data for precision samples is recorded in Table 20 of FIG. 34, Graph 8 of FIG. 35, Graph 9 of FIG. 36, and Graph 10 of FIG. 37. Ct data for positive precision pool sample S20210112-00320 did not meet acceptance criteria with three runs, so an additional run was performed (Pool Run 4) on all precision pools. The pooling method is considered valid as acceptance criteria were met for all samples for three of the four testing days.

Finally, the pooled samples were clinically evaluated by obtaining 15 Pool Tubes contrived using dry swabs collected from positive and negative donors. One swab per positive donor was added to dry swabs from 24 confirmed negative donors in a 50 ml conical with 6 ml of saline per pool. Expected Ct values for the dry swabs were extrapolated from the verification sample collected at each timepoint.

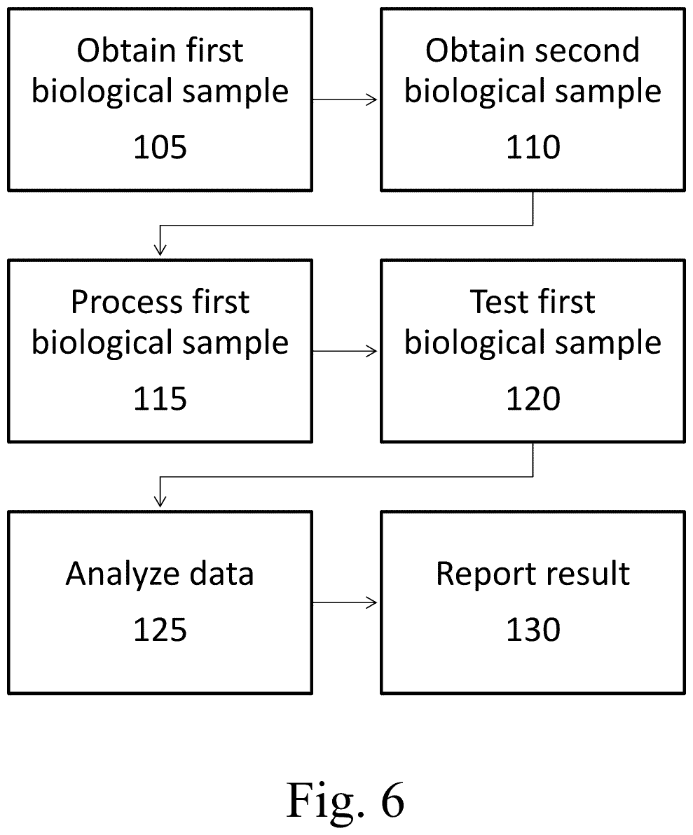

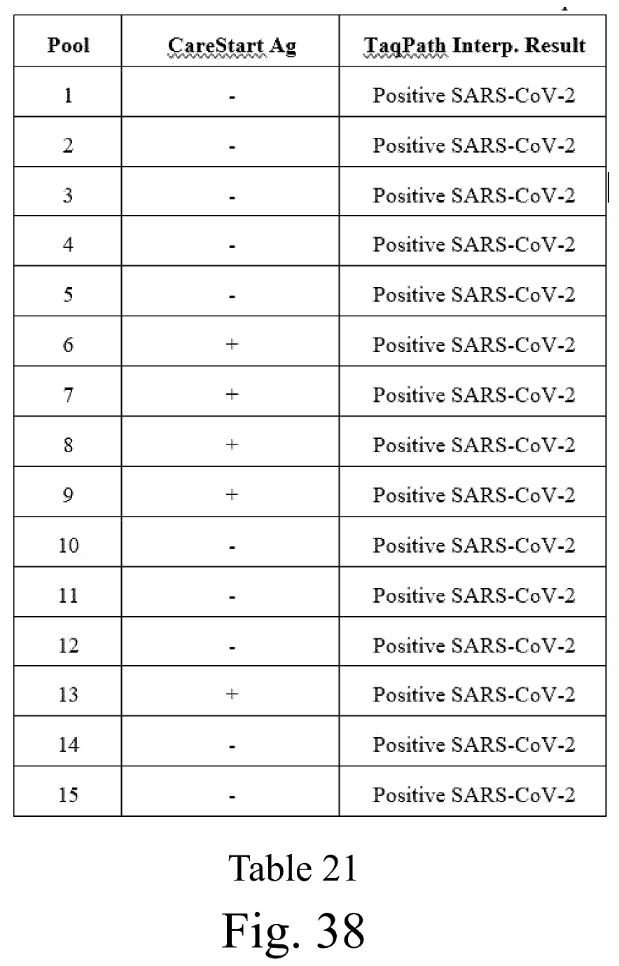

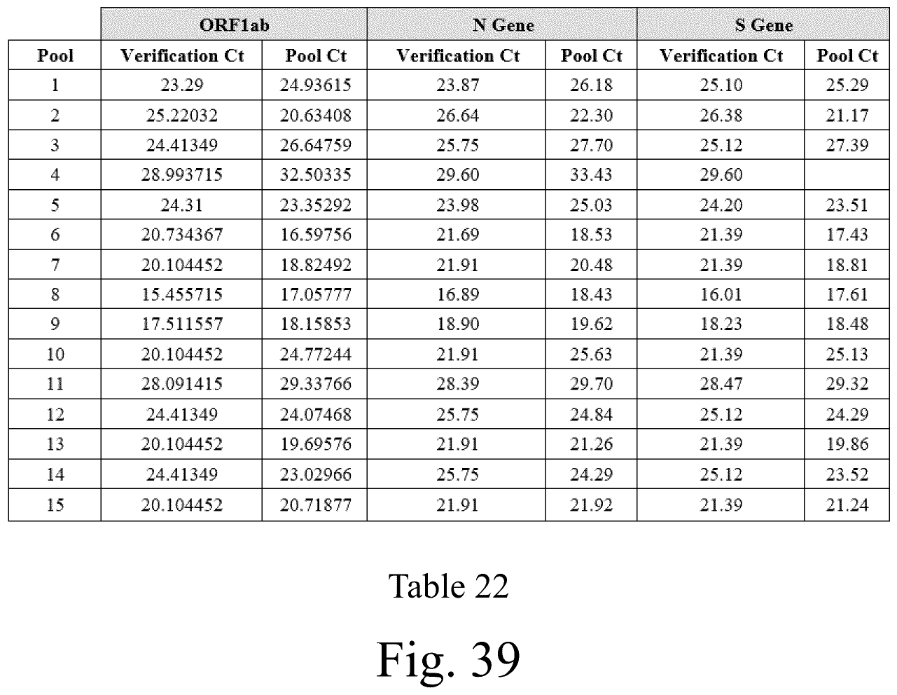

400 μl of saline from each Pool Tube was screened using the CareStart COVID-19 Rapid Antigen Test. 200 μl of media from each Pool Tube was tested using the TaqPath assay described above. As seen in Table 21 of FIG. 38, Table 22 of FIG. 39, and FIG. 6, five of fifteen contrived samples, containing a positive swab pooled with twenty-four negative swabs, tested positive for SARS-CoV-2 using the CareStart Rapid Antigen Test. As this test will be used as a screening tool to determine whether pools will reflex to pooled PCR testing or retain testing, results are acceptable for diagnostic use. All contrived samples containing a positive swab pooled with twenty-four negative swabs tested positive for SARS-CoV-2 using the TaqPath assay. As all positive pool samples will be deconvoluted and retain samples tested individually for reporting, results are acceptable for diagnostic use.

From above, testing biological samples placed in batches or pooled is a feasible and valid method of accurately screening for the presence of disease. Therefore, as shown in FIG. 6, when an individual or patient is to be tested for the presence of disease, a first biological sample 105 should be obtained for pooled sample testing, and a second biological sample 110 should also be obtained in case further testing is required. The first biological sample 105 should be processed (e.g., swirled in media to release viral particles) and combined with other biological samples to create a pool or batch 115. Once the sample pool is created, the sample pool (i.e., first biological sample, along with others) is tested 120 for the presence of a pathogen or disease. The data from the test is then analyzed 125 and the results are reported 130. If the results are negative, then the individual or community (e.g., company or school) receives the result to conclude the test. If the sample pool is positive, however, the second biological samples for each individual within a pool is tested 135 to determine which individual(s) are infected with the disease. The individual results are then reported 130 so that the individual(s) may quarantine to prevent transmission of the disease.

### VIII. Other Considerations

Originally, diagnostic nasal swab samples collected in liquid media for SARS-CoV-2 testing using the TaqPath assay require that specimens are stored at 2-8° C. for a maximum of 72 hours after collection. If a delay in testing or shipping is expected, specimens must be stored at −70° C. or below. If specimens are transported without delay, specimens may be transported in a cooler with ice packs. If a delay in transport is expected that would result in receiving of specimens more than 72 hours after collection, specimens should be store at −70° C. or below and transported on dry ice. Thus, if specimens are required to be stored at −70° C. or below and transported on dry ice, specimen transportation requires more logistics and additional costs than transporting specimens at room temperature.

Room temperature stability was assessed using two previously determined SARS-CoV-2 positive clinical samples, one sample collected in sterile saline, the other commercial viral transport media. Samples were pulled from long-term, −80° C. storage, allowed to thaw and serially diluted into 15 ml conical tubes. Diluted samples were immediately tested using the TaqPath workflow and Ct data recorded for time 0. Diluted samples were stored at room temperature and retested after 24, 48 and 72 hours.

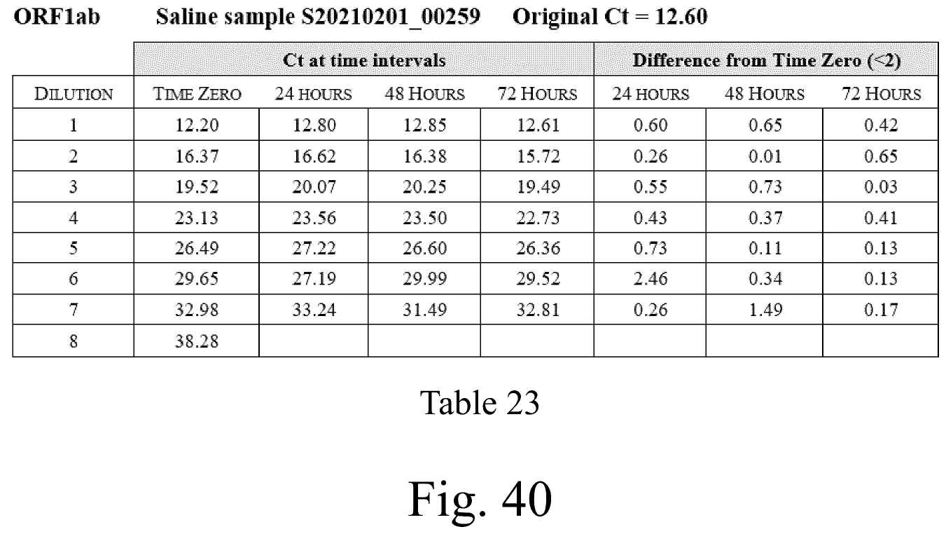

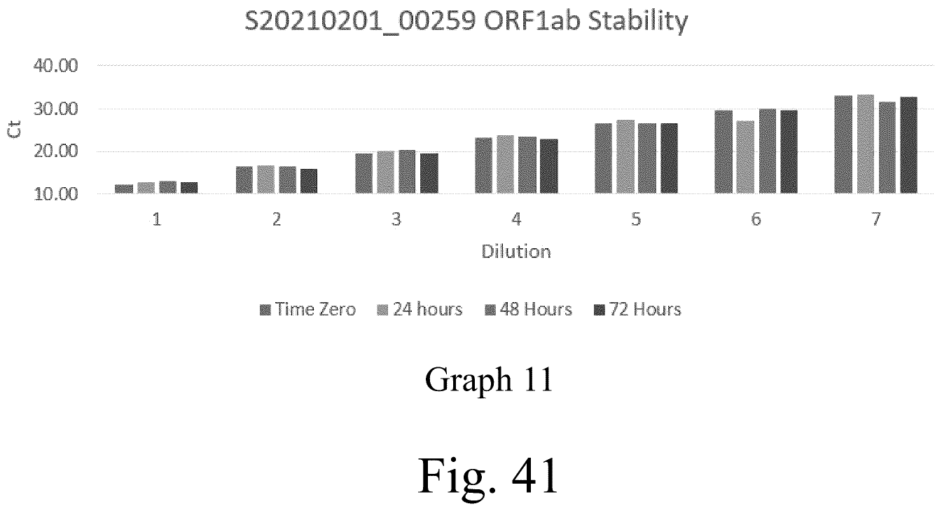

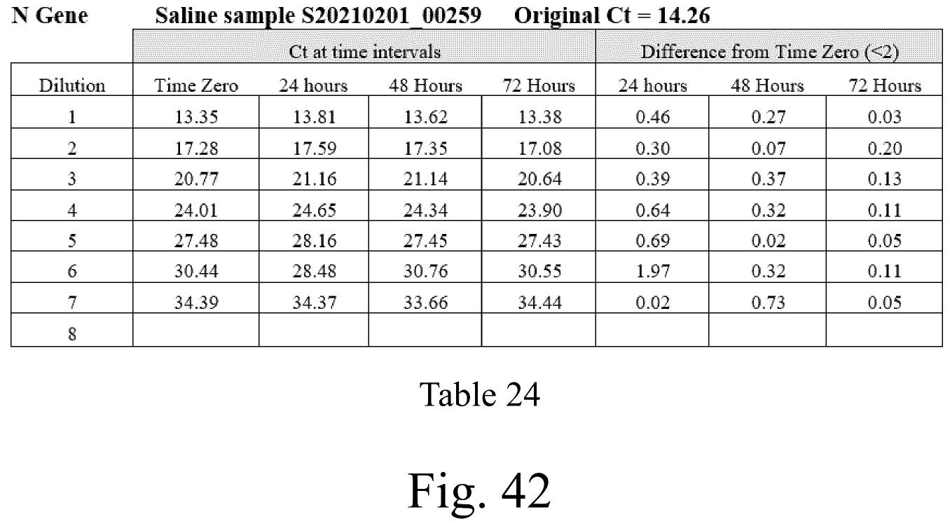

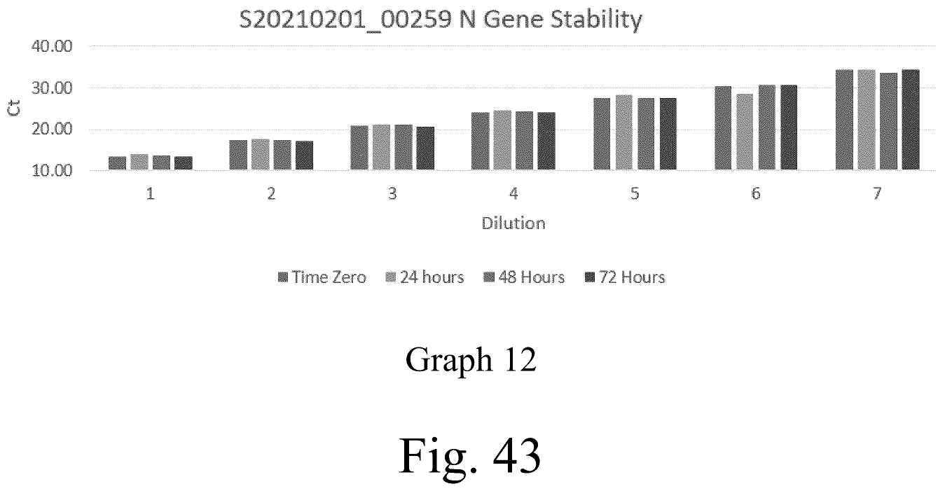

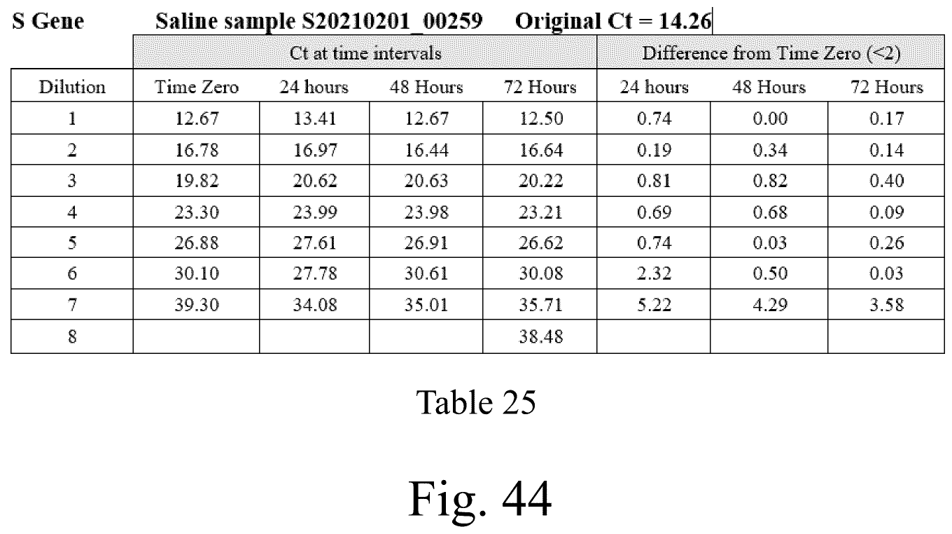

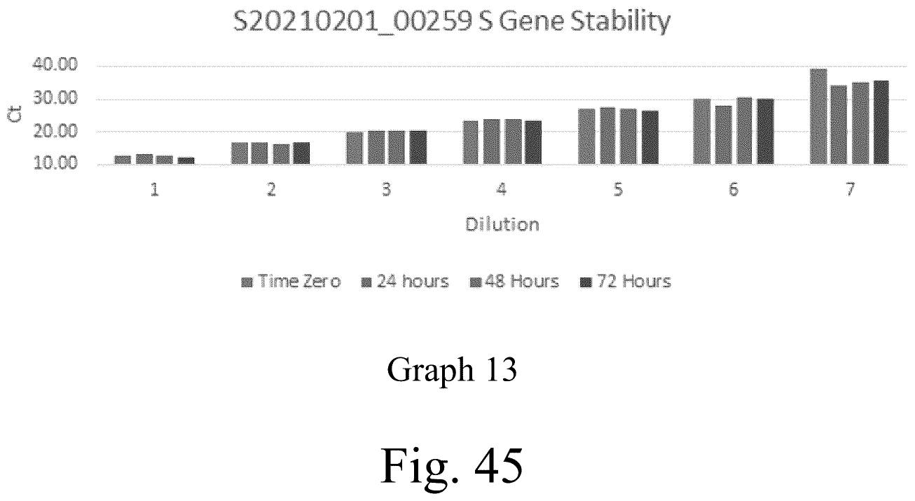

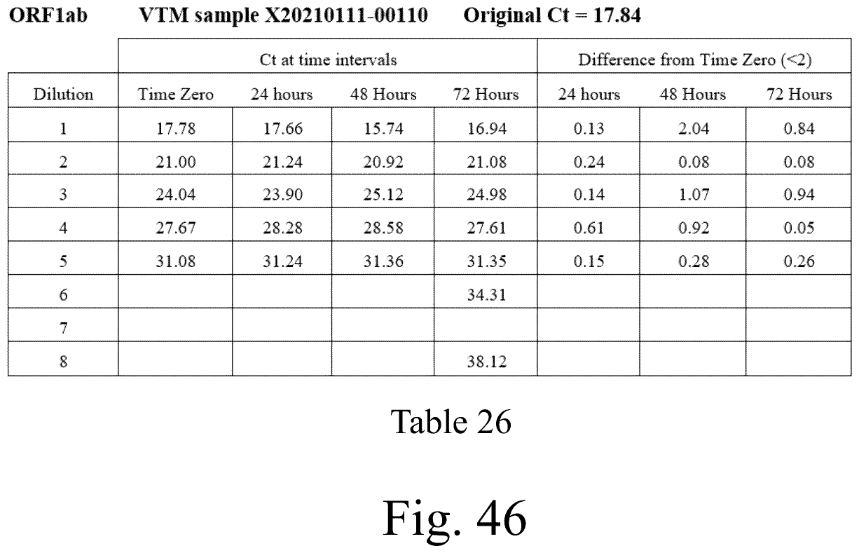

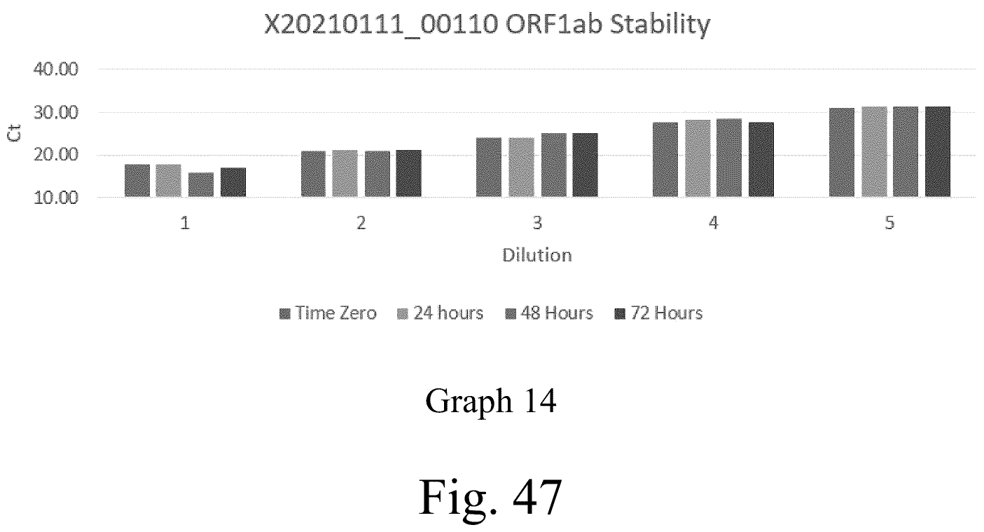

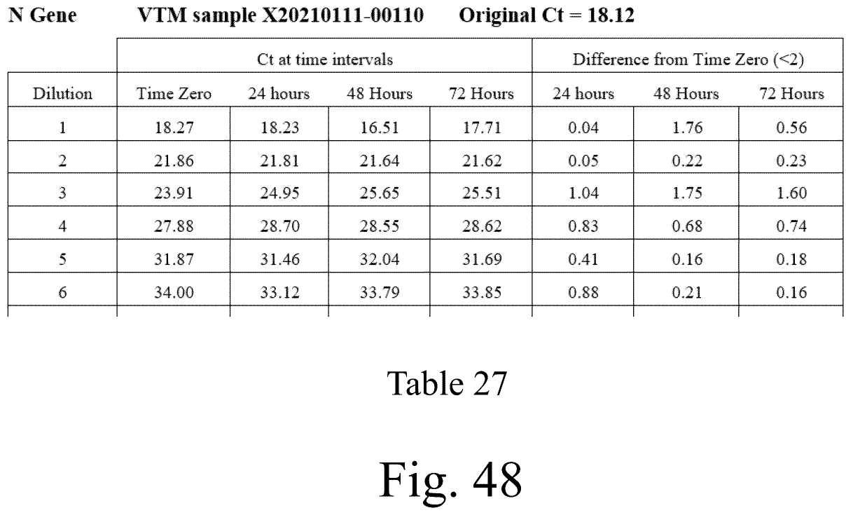

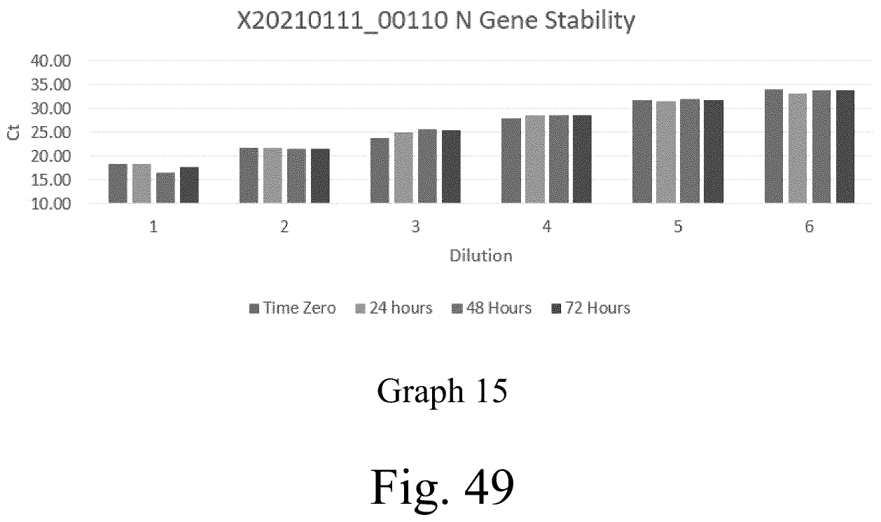

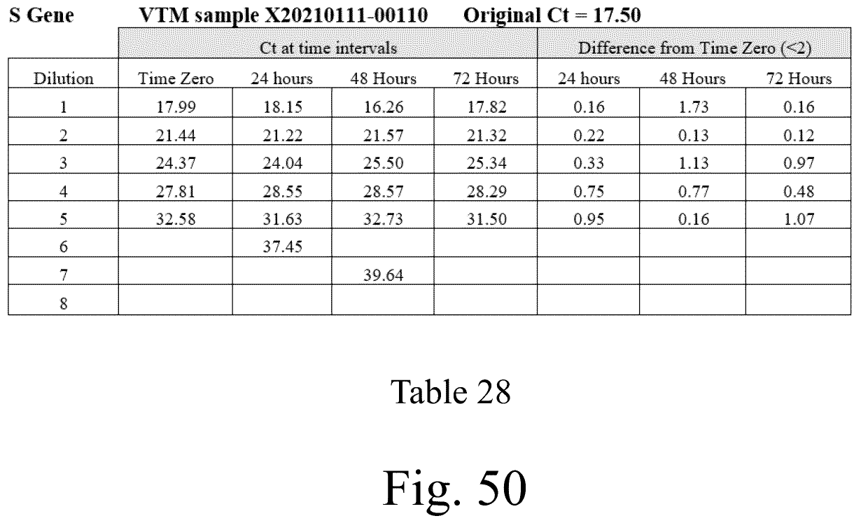

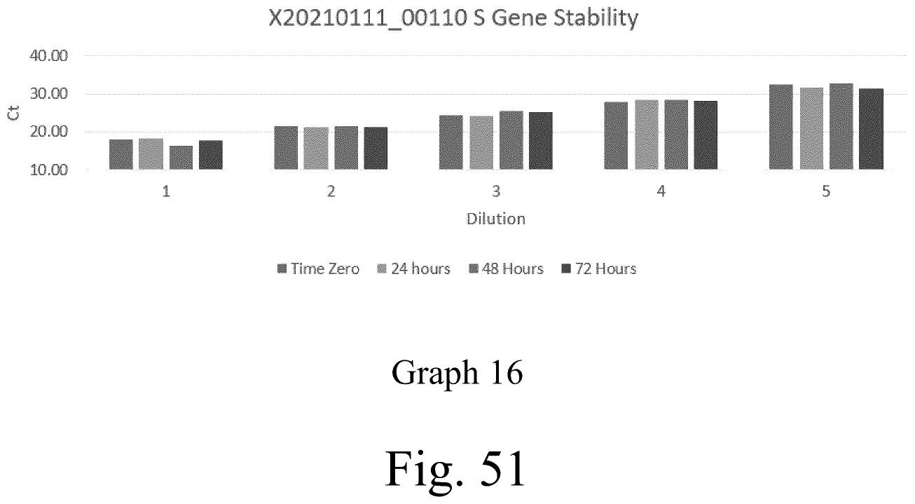

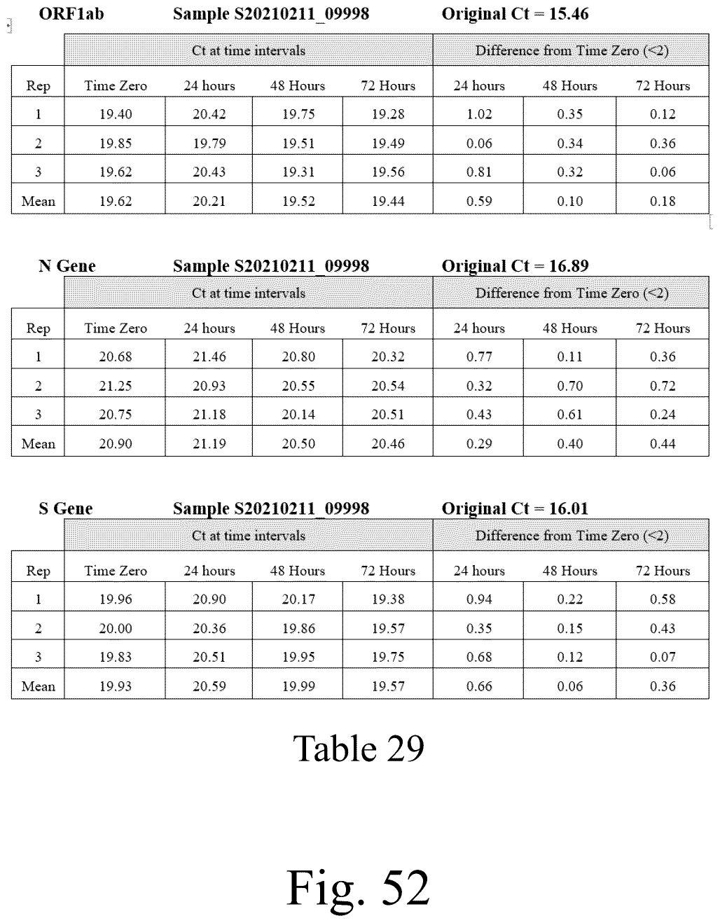

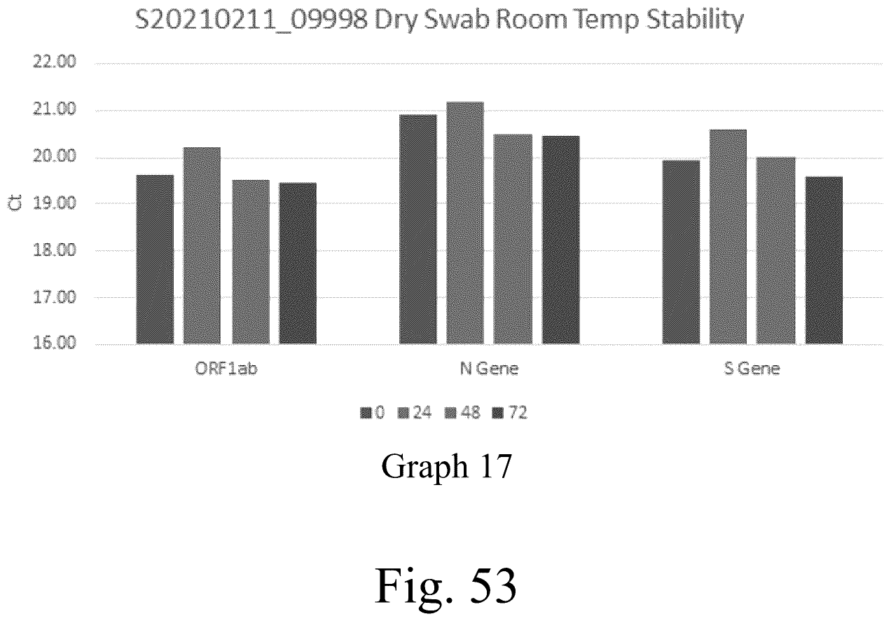

As seen in Table 23 of FIG. 40 and the tables and graphs that follow, liquid media room temperature stability was determined from Ct data. Room temperature storage is acceptable for samples collected in saline and/or viral transport media for up to 72 hours, as Ct results did not increase by >2 Cts from time zero for any samples tested.

In addition to testing sample stability for diagnostic nasal swab samples collected in liquid media, dry swab sample temperature stability was also tested. Currently, the CDC recommends if dry swabs collected for SARS-CoV-2 RT-PCR testing cannot be processed immediately following collection, they should be stored at −80° C. These storage conditions will not be feasible for samples collected in the field. Thus, it would be beneficial if dry swabs are not required to be stored at −80° C. if not immediately testing.

Dry swab room temperature stability was determined using two previously determined SARS-CoV-2 positive clinical samples, collected in sterile saline, with starting Ct values of −16 and −21. The clinical samples were pulled from long-term, −80° C. storage and allowed to thaw. A dry swab was placed into each sample and allowed to fully saturate. Excess liquid was released along the side of the sample tube. This was repeated for a total of 12 swabs per saline sample. Triplicate dry swabs per saline sample were eluted in 1 ml of sterile saline and the saline immediately tested using the TaqPath workflow. Ct data was recorded for time 0. Dry swabs were stored at room temperature and retested after 24, 48 and 72 hours.

Room temperature stability was determined from Ct data. Room temperature storage is acceptable for dry swab samples for up to 72 hours, as Ct results did not increase by >2 Cts from time zero for any samples tested.

In order to confirm whether the protocol described above would be viable for collecting and testing pooled samples, feasibility studies using the Bovilis Coronavirus were done. To test the feasibility of concentrating large amounts of media, samples of Bovilis Coronavirus, at known concentrations across the dynamic range of the assay, were obtained. Obtained samples had 25 mL total volume and were concentrated to 1.0 mL using the InnovaPrep CP Select particle bio-concentrator as described above. Also as previously described, 200 μl of concentrated sample was transferred to a single well of a 96-well, deep-well plate for extraction. Nucleic acids were isolated and purified from the concentrated specimens using the MagMAX™ Viral/Pathogen II Nucleic Acid Isolation Kit via an automated process using the KingFisher™ Flex Purification System. The purified nucleic acid was reverse transcribed into cDNA and amplified using an RT-PCR method.

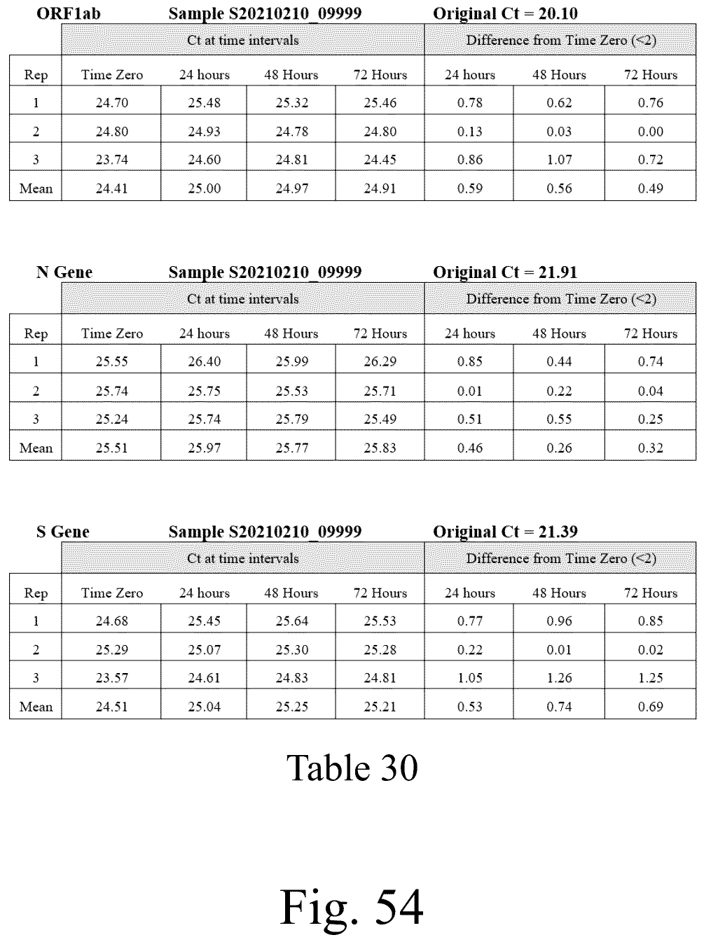

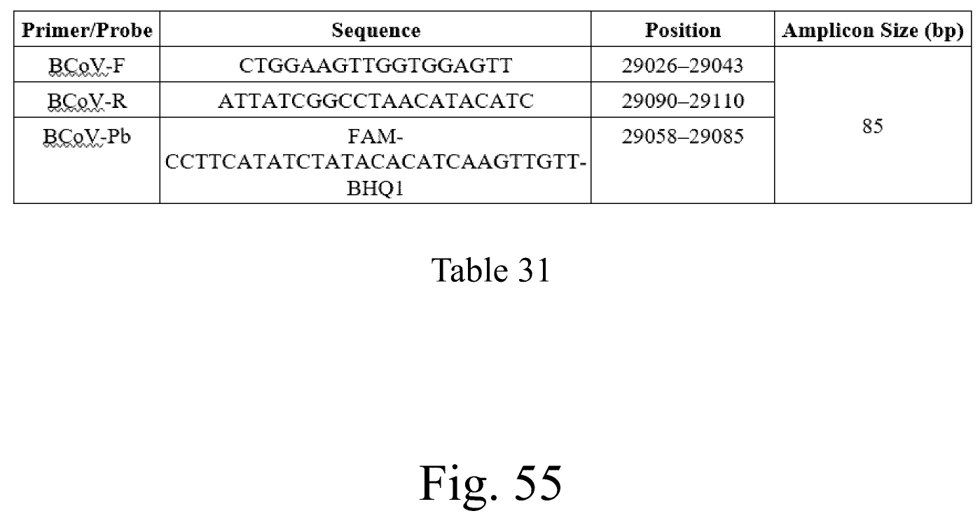

In order to extract RNA, reverse transcribe into cDNA, and amplify and detect using real-time PCR, two primers and one probe were used. The primer sequences included: (1) CTGGAAGTTGGTGGAGTT; (2) ATTATCGGCCTAACATACATC; while the probe sequence is: FAM-CCTTCATATCTATACACATCAAGTTGTT-BHQ1 as indicated in Table 31 of FIG. 54. The forward primer is located at 29026-29043, while the reverse primer is located at 29090-29110, and together allow the amplification of 85 base pairs. The probe is located at 29058-29085. To perform RT-PCR, 5 μL extracted sample+15 μL of mastermix per well was used. 900 nM of each primer and 250 nM probe (3.6:1) (18 pmol each primer, 5 pmol probe, in 20 μL final reaction volume). The reagents were transferred to a 384-well plate and samples added. RT-PCR cycling conditions included: 45° C./20 min→95° C./2 min→(95° C./15 sec→50° C./1 min) for 45 cycles.

Quantitated standards were prepared alongside samples for each concentration run to compare Ct values. Ct values indicated that the method of concentration was feasible.

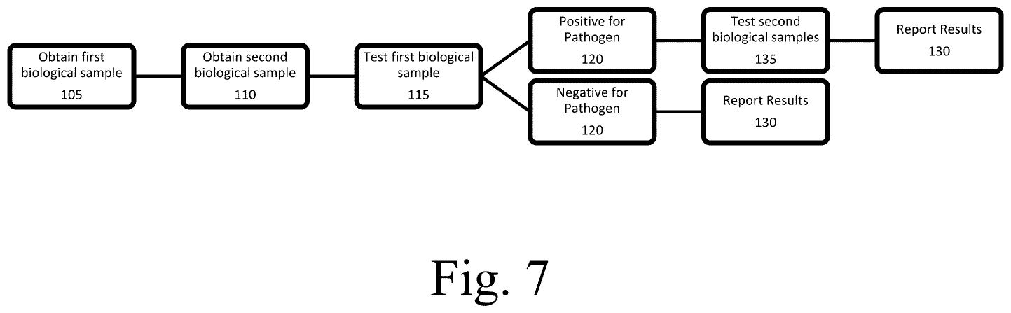

The present invention as described hereinabove, as depicted in FIGS. 1-7, and as further described elsewhere herein is advantageously directed to a method of and system for pooled pathogen testing and more particularly to a system and method of pooled coronavirus testing for testing and detecting the presence or absence of coronavirus in pooled groups of individuals in pool sizes of 25 people or persons, 50 people or persons, 75 people or persons, and/or 100 people or persons. At least one embodiment of the present invention is further directed to such a method and system of pooled pathogen testing of even larger pools having a pool size of greater than 100 individuals, preferably having a pool size of at least 200 individuals, more preferably having a pool size of at least 500 individuals, even more preferably having a pool size of at least 750 individuals, and still even more preferably having a pool size of at least 1,000 individuals. In another preferred embodiment, the present invention is even further directed to such a method and system of pooled pathogen testing of even larger pools having a pool size greater than 1,000 persons and which preferably is directed to method and system of pooled pathogen testing of even larger kilopools having a pool size of at least 2,000 persons, preferably having a pool size of at least 5,000 persons, more preferably having a pool size of at least 7,500 persons, and even more preferably having a pool size of at least 10,000 persons. In yet another preferred embodiment, the present invention is still further directed to such a method and system of pooled pathogen testing of even larger pools having a pool size greater than 10,000 individuals and which preferably is directed to method and system of pooled pathogen testing of even larger pools having a pool size of at least 15,000 individuals, preferably having a pool size of at least individuals, more preferably having a pool size of at least 50,000 individuals, even more preferably having a pool size of at least 75,000 individuals, and still even more preferably having a pool size of at least 100,000 individuals. In yet a still further preferred embodiment, the present invention is still further directed to such a method and system of pooled pathogen testing of even larger megapools having a pool size greater than 125,000 people and which preferably is directed to method and system of pooled pathogen testing of even larger pools having a pool size of at least 250,000 people, preferably having a pool size of at least 500,000 people, more preferably having a pool size of at least 750,000 people, and even more preferably having a pool size of at least 1,000,000 people.

The present invention is directed to at least one implementation of a method of pooled testing for a pathogen that includes: (a) collecting a sample from each one of at least a plurality of pairs, i.e., at least three, of individuals, i.e., members, that make up a testing pool; (b) pooling all of the samples from all of the individuals of the pool into a single pooled sample; (c) processing the pooled sample to prepare the pooled sample for performing a diagnostic test thereon; and (d) performing a diagnostic test on the processed pooled sample to obtain a test result indicative of the presence or absence of the pathogen, the test result being either (i) a positive rest result indicating the presence of the pathogen in one or more of the individuals of the testing pool, or (ii) a negative test result indicating the absence of the pathogen in any of the individuals of the testing pool. During the collection step (a) a second sample also is collected from each one of the same members of the testing pool, with this second sample stored separately from the pooled sample so that each second sample can be separately tested if the pooled sample tests positive to determine which one or more members of the testing pool test positive and carry the pathogen.

Each one of the samples can be and preferably is taken or obtained nasally, e.g., via a nasal specimen, bronchially, e.g., via a bronchial specimen, or via an oropharyngeal sample, e.g., via a throat specimen. Where the samples are obtained by taking oropharyngeal samples, the oropharyngeal samples can be nasopharyngeal samples, e.g., specimens taken from the oropharynx, oropharyngeal samples, e.g., specimens taken from the oropharynx, and/or hypopharyngeal samples, e.g., specimens taken from the hypopharynx of each person of the pool. Each one of the samples can also be a sample obtained from aspirate or a wash including from nasal aspirate, a nasal wash, nasopharyngeal aspirate, a nasopharyngeal wash, endotracheal aspirate, an endotracheal wash, a bronchoalveolar lavage (BAL), a bronchial wash, a throat wash, or another type of nasal and/or upper respiratory system wash.

In one preferred implementation of a method of pooled pathogen testing of the present invention, a swab is used to collect each one of the samples from each member of the testing pool. In one such preferred method implementation, a nasal swab is used to swab at least one of the nostrils and preferably a separate nasal swab is used to swab each one of the nostrils of each one of the individuals of the pool. If desired, swabs can be used to collect bronchial specimens, oropharyngeal specimens, as well as to collect specimens from another other biological site, i.e., in vivo site, of the members of the pool during step (a) of the method. If desired and depending upon the specific type of specimen collecting method used to obtain the samples from the members of the pool, each swab used can be a nasopharyngeal swab, a mid-turbinate swab, a foam tipped oral swab, an anterior nares/nasal swab, or an oropharyngeal swab.

In a preferred implementation of a method of pooled pathogen testing of the present invention, at least two samples are taken from each member of the testing pool, with (i) one sample of each member of the pool collectively defining a pooled sample subset of all of the samples collected from the pool members during the sampling session that are pooled together into a common pooled pathogen sample upon which a pooled pathogen test is performed to determine whether any member(s) of the pool have, e.g., are infected with, the pathogen, and (ii) another sample of each member of the pool collectively defining a pathogen verification sample subset of all of the samples collected from the pool members during the sampling session which remain separate from each other and which are stored separately from each other to each be tested to determine which member(s) of the pool have, e.g., are infected with, the pathogen upon a positive test result obtained from testing the pooled sample. At least the samples or specimens of the pathogen verification sample subset individually or separately stored, such as in a refrigerator, temperature, climate and/or humidity-controlled vault, or another type of pathogen sample storage chamber configured for stable multiple week, i.e., at least a plurality of weeks, and preferably multiple month, i.e., at least a plurality of months, sample or specimen retention, and which preferably also is lockable or otherwise secure, so that each individual sample or specimen is ready to be separately or individually tested upon a positive test result obtained from the pooled sample.

In a preferred implementation of the pooled pathogen testing method, all of the samples from each one of the members of the pool of the pooled sample subset are placed in a single container or a single bag for subsequent pooled pathogen testing on the pooled sample. In one preferred pooled pathogen testing method implementation, all of the samples from all of the pool members that makeup the pooled sample subset are placed in a single container or single bag to which no liquid, e.g., no water, nor any solution, e.g., no aqueous solution, is added. In one such preferred method implementation, the single container or single bag contains no liquid nor any solution prior to all of the samples being pooled together in the single container or single bag and no liquid nor any solution is added after all of the samples have been pooled together in the single container or single bag. All of the samples of the pooled sample subset are preferably placed in the single container or single bag during the same specimen collecting session where all of the samples are being collected from all of the pool members with all of the samples of the pooled sample subset placed in the single container or single bag while at the locus or location of the testing site where the specimen collecting session is carried out to obtain samples from all of the pool members. As such, the pooling of the samples in method step (b) is performed at a site or location where the samples are taken from the members of the testing pool that preferably is a common site or location where all of the samples are taken from all of the members of the pool during the same sample collecting session.

In a preferred pooled sample pathogen testing method implementation, the sample collecting step (a) includes performing the following substeps for each individual of the testing pool: (1) collecting a biological sample from the individual using a sample collector that is placed in a sample holding fluid medium (viral transport medium) in a sterile sample holding container, (2) transferring at least part of the biological sample from the sample collector to the sample holding fluid media in the sample holding container, and (3) sealing the sample holding container. A preferred sample collector is or includes a swab that is used to swab by frictionally engaging at least one and preferably both nostrils of the individual of the pool whose sample is being taken, the sample holding fluid media is or includes a saline solution in the sample holding container, during transferring of at least part of the sample from the swab to the saline solution, at least one of the swab, saline solution and sample holding container are agitated to cause transfer of at least part of the sample from the swab to the saline solution in the sample holding container, and thereafter the swab is removed from the sample holding container before the container is sealed. In a preferred sample collecting method implementation, the swab, the saline solution and the sample holding container are all agitated together or substantially simultaneously in preparing or readying the pooled sample for diagnostic or pathogen testing.

In one preferred implementation of the sample collecting step (a), the following substeps are performed:
a.1. After swabbing both nostrils of the individual of the pool, remove the cap from a 15 ml conical tube containing liquid saline solution media and place the swab into the saline solution media;
a.2. Vigorously swirl the swab in the media for at least 10 seconds to transfer at least some of the biological material from the individual to the saline solution media;
a.3. Slowly pull the swab out of the saline solution media until the swab is resting on the inside of the tube where no saline solution media is in contact with the swab;
a.4. With pressure, twirl the swab against the inside of the tube to squeeze as much liquid as possible out of the end of the swab and into the tube;
a.5. Discard the used swab in an appropriate waste container;
a.6. Replace the cap on the tube; and
a.7. Tighten the cap until secure.

In another preferred implementation of the sample collecting step (a), the following substeps are performed:
Swab 1
   a.1. After swabbing both nostrils, place dry swab into an empty, sterile 50 ml tube that preferably is a 50 ml conical tube; and
   a.2. Continue adding swabs collected from additional patients for a maximum of 25 swabs per 50 ml tube.
Swab 2 (required for confirming positive and inconclusive pool results):
   a.3. After swabbing both nostrils, remove the cap from a 15 ml conical tube with saline and place the swab into the media;
   a.4. Vigorously swirl the swab in the media. Perform this swirling for at least 10 seconds;
   a.5. Slowly pull the swab out of the liquid until it is resting on the inside of the tube with no media around it;
   a.6. With pressure, twirl the swab against the inside of the tube to squeeze as much liquid as possible out of the end of the swab and into the tube;
   a.7. Discard the used swab in an appropriate waste container;
   a.8. Replace the cap on the tube; and
   a.9. Tighten the cap until secure.

In a further preferred implementation of the sample collecting step (a), the following substeps are performed:
Swab 1
   a.1. After swabbing both nostrils, place dry swab into an empty, sterile ml tube that preferably is a 100 ml conical tube; and
   a.2. Continue adding swabs collected from additional patients for a maximum of 50 swabs per 100 ml tube.
Swab 2 (required for confirming positive and inconclusive pool results):
   a.3. After swabbing both nostrils, remove the cap from a 15 ml conical tube with saline and place the swab into the media;
   a.4. Vigorously swirl the swab in the media. Perform this swirling for at least 10 seconds;
   a.5. Slowly pull the swab out of the liquid until it is resting on the inside of the tube with no media around it;
   a.6. With pressure, twirl the swab against the inside of the tube to squeeze as much liquid as possible out of the end of the swab and into the tube;
   a.7. Discard the used swab in an appropriate waste container;
   a.8. Replace the cap on the tube; and
   a.9. Tighten the cap until secure.

In a still further preferred implementation of the sample collecting step (a), the following substeps are performed:
Swab 1
   a.1. After swabbing both nostrils, place dry swab into an empty, sterile 150 ml tube that preferably is a 150 ml conical tube; and
   a.2. Continue adding swabs collected from additional patients for a maximum of 75 swabs per 150 ml tube.
Swab 2 (required for confirming positive and inconclusive pool results)
   a.3. After swabbing both nostrils, remove the cap from a 15 ml conical tube with saline and place the swab into the media; and
   a.4. Vigorously swirl the swab in the media. Perform this swirling for at least 10 seconds; and
   a.5. Slowly pull the swab out of the liquid until it is resting on the inside of the tube with no media around it; and
   a.6. With pressure, twirl the swab against the inside of the tube to squeeze as much liquid as possible out of the end of the swab and into the tube; and
   a.7. Discard the used swab in an appropriate waste container; and
   a.8. Replace the cap on the tube; and
   a.9. Tighten the cap until secure.

In yet another preferred implementation of the sample collecting step (a), the following substeps are performed:
Swab 1
   a.1. After swabbing both nostrils, place dry swab into an empty, sterile 200 ml tube that preferably is a 200 ml conical tube; and
   a.2. Continue adding swabs collected from additional patients for a maximum of 100 swabs per 200 ml tube.
Swab 2 (required for confirming positive and inconclusive pool results)
   a.3. After swabbing both nostrils, remove the cap from a 15 ml conical tube with saline and place the swab into the media; and
   a.4. Vigorously swirl the swab in the media. Perform this swirling for at least 10 seconds; and
   a.5. Slowly pull the swab out of the liquid until it is resting on the inside of the tube with no media around it; and
   a.6. With pressure, twirl the swab against the inside of the tube to squeeze as much liquid as possible out of the end of the swab and into the tube; and
   a.7. Discard the used swab in an appropriate waste container; and
   a.8. Replace the cap on the tube; and
   a.9. Tighten the cap until secure.

In still yet another preferred implementation of the sample collecting step (a), the following substeps are performed for each member of a pool having a pool size of between 150 and 200 individuals:
Swab 1
   a.1. After swabbing at least one and preferably both nostrils of the pool member, place the dry swab into an empty, sterile 400 ml tube that preferably is a 400 ml conical tube;
   a.2. Continue adding swabs collected from additional patients until the specimens or samples from at least 150 swabs and no more than maximum of 200 swabs are added to the 400 ml tube;
Swab 2 (required for confirming positive and inconclusive pool results)
   a.3. After swabbing both nostrils, remove the cap from a 15 ml conical single sample or specimen holding tube with saline and place the swab into the media;
   a.4. Vigorously swirl the swab in the media in the single specimen holding tube and continue doing so for at least 10 seconds;
   a.5. Slowly pull the swab out of the liquid in the single specimen holding tube until it is resting on the inside of the tube with no media around it;
   a.6. With pressure, twirl the swab against the inside of the single specimen holding tube to squeeze as much liquid as possible out of the end of the swab and into the tube;
   a.7. Discard the used swab in an appropriate waste container; and
   a.8. Replace the cap on the single specimen holding tube; and
   a.9. Tighten the cap until secured to the single specimen holding tube.

In still yet another preferred implementation of the sample collecting step (a), the following substeps are performed for each member of a pool having a pool size of between 150 and 200 individuals:
Swab 1
   a. After swabbing both nostrils, place dry swab into an empty, sterile 400 ml tube that preferably is a 400 ml conical tube; and
   b. Continue adding swabs collected from additional patients for a minimum of 200 swabs and a maximum of 250 swabs per 400 ml tube.
Swab 2 (required for confirming positive and inconclusive pool results)
   c. After swabbing both nostrils, remove the cap from a 15 ml conical tube with saline and place the swab into the media; and
   d. Vigorously swirl the swab in the media. Perform this swirling for at least 10 seconds; and
   e. Slowly pull the swab out of the liquid until it is resting on the inside of the tube with no media around it; and
   f. With pressure, twirl the swab against the inside of the tube to squeeze as much liquid as possible out of the end of the swab and into the tube; and
   g. Discard the used swab in an appropriate waste container; and
   h. Replace the cap on the tube; and
   i. Tighten the cap until secure.

In carrying out a preferred implementation of the pool pathogen testing method, the sample processing step (c) includes performing the following substeps: (1) preparing the pooled sample containing sample from each one of the individuals of the testing pool, (2) extracting ribonucleic acid (RNA) from cells in the pooled sample, (3) purifying the pooled RNA, and (4) eluting the purified pooled RNA. In at least one pathogen testing method implementation, the sample processing step (c) further includes performing the following additional substep of producing a diagnostic testing ready purified pooled RNA containing mixture composed of (i) the purified pooled RNA and (ii) a solution that includes a buffer, reverse transcriptase, nucleotides (dNTPs), a forward primer, a reverse primer, a probe, and a DNA polymerase, and wherein the diagnostic testing step (d) includes performing a PCR diagnostic test using the diagnostic testing ready purified pooled RNA containing mixture. The present invention therefore includes a PCR diagnostic test that uses the diagnostic testing ready purified pooled RNA containing mixture in determining whether the pooled sample is positive or negative in carrying out the diagnostic testing step (d).

The PCR diagnostic test preferably is configured to determine whether a coronavirus, preferably SARS-CoV-2 genetic material, is present in any of the RNA in the diagnostic testing ready purified pooled RNA containing mixture during carrying out step (d). In a preferred embodiment and method implementation, the PCR diagnostic test is configured to determine whether a sufficient amount of SARS-CoV-2 genetic material is present in the RNA in the diagnostic testing ready purified pooled RNA containing mixture to produce a positive PCR diagnostic test result in step (d). In another preferred embodiment, the PCR diagnostic test is configured to determine whether a great enough viral load of SARS-CoV-2 is present to produce a positive PCR diagnostic test result in step (d). In one such preferred embodiment, the PCR diagnostic test performed during the diagnostic pathogen testing step (d) is an RT-PCR diagnostic test performed using the diagnostic testing ready purified pooled RNA containing mixture in determining whether the test yields a positive or negative result.

In a preferred pooled testing method implementation of the present invention, the sample processing step (c) encompasses performing the following substeps: (1) lysing cells in the pooled sample to extract RNA therefrom, (2) binding the pooled RNA lysate to magnetic binding beads, (3) washing the magnetic binding beads to which the pooled RNA is bound to purify the bound pooled RNA, and (4) eluting the purified pooled RNA from the magnetic binding beads. In one such method implementation, in the binding substep (2), the sample containing the RNA lysate is mixed with a proteinase K, a MS2 phage control, a bead-binding solution, and the magnetic binding beads during incubation of the RNA lysate to thereby bind the RNA in the lysate to the magnetic binding beads. In such a method implementation, during the binding substep (2), the sample containing the RNA lysate is mixed with proteinase K, MS2 phage control, bead-binding solution, and magnetic binding beads during the incubation of the RNA lysate thereby binding the RNA in the lysate to the magnetic binding beads. In a preferred test embodiment and testing method implementation, the binding solution includes thiocyanic acid and guanidine, preferably is composed of thiocyanic acid and guanidine, more preferably essentially consists of thiocyanic acid and guanidine, and even more preferably consists of thiocyanic acid and guanidine. Incubation preferably is performed at an incubation temperature that is elevated incubation temperature that is a temperature greater than the temperature at which lysing was performed in substep (1) above.

In another preferred pooled testing method implementation, the diagnostic testing ready purified pooled RNA containing mixture is composed of (i) the purified pooled RNA and (ii) a solution comprised of a buffer, reverse transcriptase, nucleotides (dNTPs), forward primer, reverse primer, a probe, and DNA polymerase, and wherein during the diagnostic testing step (d) the PCR diagnostic test is performed with or using the diagnostic testing ready purified pooled RNA containing mixture. The sample processing step (c) further includes performing the following additional substep of producing a diagnostic testing ready purified pooled RNA containing mixture that includes (i) the purified pooled RNA and (ii) a solution comprised of a buffer, reverse transcriptase, nucleotides (dNTPs), forward primer, reverse primer, a probe, and DNA polymerase, and wherein the diagnostic testing step (d) is, includes or encompasses performing a PCR diagnostic test with or using the diagnostic testing ready purified pooled RNA containing mixture.

In one such preferred pooled testing method implementation, the diagnostic testing ready purified pooled RNA containing mixture is consists essentially of (i) the purified pooled RNA and (ii) a solution comprised of a buffer, reverse transcriptase, nucleotides (dNTPs), forward primer, reverse primer, a probe, and DNA polymerase, and wherein during the diagnostic testing step (d) the PCR diagnostic test is performed with or using the diagnostic testing ready purified pooled RNA containing mixture. The sample processing step (c) further includes performing the following additional substep of producing a diagnostic testing ready purified pooled RNA containing mixture that consists essentially of (i) the purified pooled RNA and (ii) a solution comprised of a buffer, reverse transcriptase, nucleotides (dNTPs), forward primer, reverse primer, a probe, and DNA polymerase, and wherein the diagnostic testing step (d) encompasses and preferably consists essentially of performing a PCR diagnostic test with or using the diagnostic testing ready purified pooled RNA containing mixture.

In another such preferred pooled testing method implementation, the diagnostic testing ready purified pooled RNA containing mixture consists of (i) the purified pooled RNA and (ii) a solution comprised of a buffer, reverse transcriptase, nucleotides (dNTPs), forward primer, reverse primer, a probe, and DNA polymerase, and wherein during the diagnostic testing step (d) the PCR diagnostic test is performed with or using the diagnostic testing ready purified pooled RNA containing mixture. The sample processing step (c) further includes performing the following additional substep of producing a diagnostic testing ready purified pooled RNA containing mixture that consists of (i) the purified pooled RNA and (ii) a solution comprised of a buffer, reverse transcriptase, nucleotides (dNTPs), forward primer, reverse primer, a probe, and DNA polymerase, and wherein the diagnostic testing step (d) encompasses and preferably consists of performing a PCR diagnostic test with or using the diagnostic testing ready purified pooled RNA containing mixture.

Such a PCR diagnostic test preferably also is configured to determine whether a coronavirus, preferably SARS-CoV-2 genetic material, is present in any of the RNA in the diagnostic testing ready purified pooled RNA containing mixture during carrying out step (d). In a preferred embodiment and method implementation, the PCR diagnostic test is configured to determine whether a sufficient amount of SARS-CoV-2 genetic material is present in the RNA in the diagnostic testing ready purified pooled RNA containing mixture to produce a positive PCR diagnostic test result in step (d). In another preferred embodiment, the PCR diagnostic test is configured to determine whether a great enough viral load of SARS-CoV-2 is present to produce a positive PCR diagnostic test result in step (d). In one such preferred embodiment, the PCR diagnostic test performed during the diagnostic pathogen testing step (d) is an RT-PCR diagnostic test performed using the diagnostic testing ready purified pooled RNA containing mixture in determining whether the test yields a positive or negative result.

In one preferred implementation of the pooled pathogen testing method, step (d) of performing a diagnostic test on the pooled sample also is done at the same site or location where the samples are collected from all of the members of the pool. In one such preferred method implementation, at least one and preferably a plurality of (i) the sample or specimen collection, (ii) the pooling of the pooled samples, (iii) the testing of the pooled sample, and (iv) performing pathogen verification testing if the pooled sample tests positive for the pathogen are performed at and preferably onboard a vehicle, such as a mobile testing lab or another type of mobile testing vehicle, preferably at the site or location where the samples are or were taken. In another such preferred implementation, at least a plurality of pairs, i.e., at least three of (i)-(iv) are performed at and preferably onboard a mobile testing vehicle, preferably mobile test lab. In still another such preferred implementation, all of (i)-(iv) are performed at and preferably onboard a mobile testing vehicle, preferably wheeled mobile test lab. In another preferred implementation of the pooled pathogen testing method, the pathogen diagnostic test is performed in step (d) at a testing site located remote from the site or location where the samples were collected from the pool members preferably by being located at least a plurality of miles or kilometers away therefrom.

In one such preferred pooled pathogen testing method implementation the samples from each one of the individuals of the pool that define the pooled sample are placed in the single container or single bag without adding any liquid, e.g., without adding any water, to the single container or single bag and without adding any solution, e.g., without adding any aqueous solution, to the single container or single bag. In such a preferred method implementation, the single container or single bag contains no liquid nor any solution prior to each one of the samples taken from each one of the individuals or members of the pool being added to the single container or single bag to pool together all of the samples from all of the individuals or members of the pool defining the pooled subset sample.

The above-described and illustrated method and system of pooled pathogen testing of the present invention can be used to pathogen test or screen even larger pools having a pool size of greater than 100 individuals, preferably having a pool size of at least 200 individuals, more preferably having a pool size of at least 500 individuals, and even more preferably having a pool size of at least 1,000 individuals. In a preferred embodiment, the present invention is even further directed to such a method and system of pooled pathogen testing of even larger kilo pools having a pool size greater than 1,000 individuals and which preferably is directed to method and system of pooled pathogen testing of even larger pools having a pool size of at least 2,000 individuals, preferably having a pool size of at least 5,000 individuals, more preferably having a pool size of at least 7,500 individuals, and even more preferably having a pool size of at least 10,000 individuals. In another preferred embodiment, the present invention is still further directed to such a method and system of pooled pathogen testing of even larger pools having a pool size greater than 10,000 individuals and which preferably is directed to method and system of pooled pathogen testing of even larger megapools having a pool size of at least 15,000 individuals, preferably having a pool size of at least 20,000 individuals, more preferably having a pool size of at least 50,000 individuals, and even more preferably having a pool size of at least 100,000 individuals.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes. Other embodiments and uses of the invention will be apparent to those skilled in the art from consideration from the specification and practice of the invention disclosed herein. It is understood that the invention is not confined to the specific reagents, formulations, reaction conditions, etc., herein illustrated and described, but embraces such modified forms thereof as come within the scope of the following claims.

What is claimed is:

1. A method of pooled testing for a pathogen comprising:
(a) collecting a sample from each one of at least a plurality of pairs of individuals comprising a testing pool;
(b) pooling all of the samples from all of the individuals of the pool into a single pooled sample;
(c) processing the pooled sample to prepare the pooled sample for performing a diagnostic test thereon; and
(d) performing a diagnostic test on the processed pooled sample to obtain a test result indicative of the presence or absence of the pathogen, the test result comprising either (i) a positive rest result indicating the presence of the pathogen in one or more of the individuals of the testing pool, or (ii) a negative test result indicating the absence of the pathogen in any of the individuals of the testing pool.

2. The method of claim 1, wherein each one of the samples comprises one of a nasal specimen, a throat specimen, or a bronchial specimen.

3. The method of claim 1, wherein each one of the samples is a sample obtained from nasal aspirate, a nasopharyngeal aspirate, an endotracheal aspirate, a bronchoalveolar lavage (BAL), a nasal wash, a bronchial wash, or a throat wash.

4. The method of claim 1, wherein a swab is used to collect each one of the samples from each one of the individuals of the testing pool.

5. The method of claim 4, wherein each swab is a nasal swab that is used to swab at least one of the nostrils of each one of the individuals of the testing pool.

6. The method of claim 5, wherein each swab is a nasal swab that is used to swab both of the nostrils of each one of the individuals of the testing pool.

7. The method of claim 4, wherein each swab is one of a nasopharyngeal swab, a mid-turbinate swab, a foam tipped oral swab, an anterior nares/nasal swab and an oropharyngeal swab.

8. The method of claim 1, wherein the samples from each one of the individuals of the pool are placed in a single container or a bag.

9. The method of claim 8, wherein the samples from each one of the individuals of the pool are placed in the single container or bag without adding any liquid or other solution in the container or bag.

10. The method of claim 9, wherein a swab is used to collect each one of the samples from each one of the individuals of the testing pool.

11. The method of claim 10, wherein each swab is one of a nasopharyngeal swab, a mid-turbinate swab, a foam tipped oral swab, an anterior nares/nasal swab and an oropharyngeal swab.

12. The method of claim 10, wherein each swab is a nasal swab that is used to swab at least one of the nostrils of each one of the individuals of the testing pool.

13. The method of claim 12, wherein each swab is a nasal swab that is used to swab both of the nostrils of each one of the individuals of the testing pool.

14. The method of claim 12, wherein each swab is one of a nasopharyngeal nasal swab, a mid-turbinate swab, a foam tipped oral swab, and an anterior nares/nasal swab.

15. The method of claim 8, wherein the samples from each one of the individuals of the pool are placed dry in the single container or bag without adding any liquid or other solution in or to the container or bag.

16. The method of claim 15, wherein a swab is used to collect each one of the samples from each one of the individuals of the testing pool.

17. The method of claim 16, wherein each swab is one of a nasopharyngeal swab, a mid-turbinate swab, a foam tipped oral swab, an anterior nares/nasal swab and an oropharyngeal swab.

18. The method of claim 16, wherein each swab is a nasal swab that is used to swab at least one of the nostrils of each one of the individuals of the testing pool.

19. The method of claim 18, wherein each swab is a nasal swab that is used to swab both of the nostrils of each one of the individuals of the testing pool.

20. The method of claim 18, wherein each swab is one of a nasopharyngeal swab, a mid-turbinate swab, a foam tipped oral swab, and an anterior nares/nasal swab.

21. The method of claim 1, wherein during step (a), the step of collecting a second sample from each one of the plurality of individuals of the testing pool that is kept separate from the pooled sample; and wherein when the test result of the pooled sample is a positive test result, performing a separate diagnostic test on each one of the second samples to determine which one or more individuals of the testing pool have a positive test result that caused the positive test result of the pooled sample.

22. The method of claim 1, wherein the pooling of the samples in step (b) is performed at a site or location where the samples are taken from the individuals of the testing pool.

23. The method of claim 1, wherein the pooling of the samples in step (b) is performed at a site or location where the sample processing step (c) and the diagnostic testing step (d) are performed.

24. The method of claim 1, wherein the sample processing step (c) comprises performing the following substeps: (1) preparing the pooled sample containing sample from each one of the individuals of the testing pool, (2) extracting ribonucleic acid (RNA) from cells in the pooled sample, (3) purifying the pooled RNA, and (4) eluting the purified, pooled RNA.

25. The method of claim 24, wherein the sample processing step (c) further comprises performing the following additional substep of producing a diagnostic testing ready, purified pooled RNA containing mixture comprised of (i) the purified pooled RNA and (ii) a solution comprised of a buffer, reverse transcriptase, nucleotides (dNTPs), forward primer, reverse primer, a probe, and DNA polymerase, and wherein the diagnostic testing step (d) comprises performing a PCR diagnostic test with or using the diagnostic testing ready, purified pooled RNA containing mixture.

26. The method of claim 25, wherein the PCR diagnostic test is configured to determine whether a coronavirus is present in any of the RNA in the diagnostic testing ready, purified pooled RNA containing mixture.

27. The method of claim 26, wherein the PCR diagnostic test is configured to determine whether SARS-CoV-2 genetic material is present in any of the RNA in the diagnostic testing ready, purified pooled RNA containing mixture.

28. The method of claim 26, wherein the PCR diagnostic test is configured to determine whether a sufficient amount of SARS-CoV-2 genetic material is present in the RNA in the diagnostic testing ready, purified pooled RNA containing mixture to produce a positive PCR diagnostic test result.

29. The method of claim 26, wherein the PCR diagnostic test is configured to determine whether a great enough viral load of SARS-CoV-2 is present to produce a positive PCR diagnostic test result.

30. The method of claim 29, wherein the PCR diagnostic test performed during the diagnostic step (d) is an RT-PCR diagnostic test performed with or using the diagnostic testing ready, purified pooled RNA containing mixture.

31. The method of claim 1, wherein the sample processing step (c) comprises performing the following substeps: (1) lysing cells in the pooled sample to extract RNA therefrom, (2) binding the pooled RNA lysate to magnetic binding beads, (3) washing the magnetic binding beads to which the pooled RNA is bound to purify the bound pooled RNA, and (4) eluting the purified pooled RNA from the magnetic binding beads.

32. The method of claim 31, wherein in the binding substep (2), the sample containing the RNA lysate is mixed with proteinase K, MS2 phage control, a bead-binding solution, and the magnetic binding beads during incubation of the RNA lysate thereby binding the RNA in the lysate to the magnetic binding beads.

33. The method of claim 32, wherein incubation is performed at an elevated incubation temperature greater than the temperature at which lysing was performed.

34. The method of claim 32, wherein the binding solution is comprised of thiocyanic acid and guanidine.

35. The method of claim 31, wherein the sample processing step (c) further comprises performing the following additional substep of producing a diagnostic testing ready, purified pooled RNA containing mixture comprised of (i) the purified pooled RNA and (ii) a solution comprised of a buffer, reverse transcriptase, nucleotides (dNTPs), forward primer, reverse primer, a probe, and DNA polymerase, and wherein the diagnostic testing step (d) comprises performing a PCR diagnostic test with or using the diagnostic testing ready, purified pooled RNA containing mixture.

36. The method of claim 35, wherein the PCR diagnostic test is configured to determine whether a coronavirus is present in any of the RNA in the diagnostic testing ready, purified pooled RNA containing mixture.

37. The method of claim 36, wherein the PCR diagnostic test is configured to determine whether SARS-CoV-2 genetic material is present in any of the RNA in the diagnostic testing ready, purified pooled RNA containing mixture.

38. The method of claim 36, wherein the PCR diagnostic test is configured to determine whether a sufficient amount of SARS-CoV-2 genetic material is present in the RNA in the diagnostic testing ready, purified pooled RNA containing mixture to produce a positive PCR diagnostic test result.

39. The method of claim 36, wherein the PCR diagnostic test is configured to determine whether a great enough viral load of SARS-CoV-2 is present to produce a positive PCR diagnostic test result.

40. The method of claim 39, wherein the PCR diagnostic test performed during the diagnostic step (d) is an RT-PCR diagnostic test performed with or using the diagnostic testing ready, purified pooled RNA containing mixture.

41. The method of claim 40, wherein the sample collecting step (a) comprises performing the following substeps for each individual of the testing pool: (1) collecting a biological sample from the individual using a sample collector that is placed in a sample holding fluid medium (viral transport medium) in a sterile sample holding container, (2) transferring at least part of the biological sample from the sample collector to the sample holding fluid media in the sample holding container, and (3) sealing the sample holding container.

42. The method of claim 41, wherein the sample collector comprises a swab that is used to swab at least one and preferably both nostrils of the individual of the pool whose sample is being taken, the sample holding fluid media comprises a saline solution in the sample holding container, during transferring of at least part of the sample from the swab to the saline solution, at least one of the swab, saline solution and sample holding container are agitated to cause transfer of at least part of the sample from the swab to the saline solution in the sample holding container, and thereafter the swab is removed from the sample holding container before the container is sealed.


SEQUENCE LISTING (3 sequences)

SEQ ID NO: 1 - DNA, 18 bp, Artificial Sequence
  Forward Primer located at position 29026-29043 of Coronavirus
ctggaagttggtggagtt

SEQ ID NO: 2 - DNA, 21 bp, Artificial Sequence
  Reverse Primer is located at 29090-29110 of Coronavirus
attatcggcctaacatacatc

SEQ ID NO: 3 - DNA, 28 bp, Artificial Sequence
  Probe sequence is FAM- CCTTCATATCTATACACATCAAGTTGTT-BHQ1, is located at 29058-29085, with F being in position 1, A being in position 2, M being in position 3, B being in position 34, H being in position 35, Q being in position 36 and 1 being in
ccttcatatctatacacatcaagttgtt